SEQUENCE LISTING (422 sequences)

SEQ ID NO: 1 - protein, 243 aa, synthetic construct
QVQLVEAGGGVVQPGRSLRLSCAASGFIFSGYGMHWVRQAPGKGLEWVTVIWYDGSNKYYADSVKGRFTISRDNSKNTLYLQMNSLRAEDTAVYYCARDTGDRFFDYWGQGTLVTVSSGGGGSGGGGSGGGGSEIVLTQSPATLSLSPGERATLSCRASQSVSSYLAWYQQKPGQAPRLLIYDASSRATGIPARFGGSGSGTDFTLTISSLEPEDFAVYYCQLRSNWPPYTFGQGTKLEIKTS

SEQ ID NO: 2 - protein, 248 aa, synthetic construct
QVQLVESGGGWQPGRSLRLSCSSSGFIFSDNYMYWVRQAPGKGLEWVATISDGGSYTYYPDSVKGRFTISRDNSKNTLFLQMDSLRPEDTGVYFCARGYYRYEGAMDYWGQGTPVTVSSGGGGSGGGGSGGGGSDIQLTQSPSSLSASVGDRVTITCKSSQSVLYSSNQKNYLAWYQQKPGKAPKLLIYWASTRESGVPSRFSGSGSGTDFTFTISSLQPEDIATYYCHQYLSSWTFGQGTKLEIKTS

SEQ ID NO: 3 - protein, 249 aa, synthetic construct
EVQLVESGGGLVKPGGSLRLSCVASGFIFSDNYMYWVRQTPEKRLEWVATISDGGSYTYYPDSVKGRFTISRDNAKNNLYLQMSSLKSEDTAIYYCARGYYRYEGAMDYWGQGTSVTVSSGGGGSGGGGSGGGGSNIVMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSWTFGGGTKLEIKTS

SEQ ID NO: 4 - protein, 45 aa, Homo sapiens
TTTPAPRPPTPAPTIASQPLSLRPEACRPAAGGAVHTRGLDFACD

SEQ ID NO: 5 - protein, 24 aa, Homo sapiens
IYIWAPLAGTCGVLLLSLVITLYC

SEQ ID NO: 6 - protein, 42 aa, Homo sapiens
KRGRKKLLYIFKQPFMRPVQTTQEEDGCSCRFPEEEEGGCEL

SEQ ID NO: 7 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 39 aa, Homo sapiens
IEVMYPPPYLDNEKSNGTIIHVKGKHLCPSPLFPGPSKP

SEQ ID NO: 9 - protein, 27 aa, Homo sapiens
FWVLVVVGGVLACYSLLVTVAFIIFWV

SEQ ID NO: 10 - protein, 41 aa, Homo sapiens
RSKRSRLLHSDYMNMTPRRPGPTRKHYQPYAPPRDFAAYRS

SEQ ID NO: 11 - protein, 488 aa, synthetic construct
MALPVTALLLPLALLLHAARPQVQLVEAGGGVVQPGRSLRLSCAASGFIFSGYGMHWVRQAPGKGLEWVTVIWYDGSNKYYADSVKGRFTISRDNSKNTLYLQMNSLRAEDTAVYYCARDTGDRFFDYWGQGTLVTVSSGGGGSGGGGSGGGGSEIVLTQSPATLSLSPGERATLSCRASQSVSSYLAWYQQKPGQAPRLLIYDASSRATGIPARFGGSGSGTDFTLTISSLEPEDFAVYYCQLRSNWPPYTFGQGTKLEIKTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 493 aa, synthetic construct
MALPVTALLLPLALLLHAARPQVQLVESGGGWQPGRSLRLSCSSSGFIFSDNYMYWVRQAPGKGLEWVATISDGGSYTYYPDSVKGRFTISRDNSKNTLFLQMDSLRPEDTGVYFCARGYYRYEGAMDYWGQGTPVTVSSGGGGSGGGGSGGGGSDIQLTQSPSSLSASVGDRVTITCKSSQSVLYSSNQKNYLAWYQQKPGKAPKLLIYWASTRESGVPSRFSGSGSGTDFTFTISSLQPEDIATYYCHQYLSSWTFGQGTKLEIKTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 494 aa, synthetic construct
MALPVTALLLPLALLLHAARPEVQLVESGGGLVKPGGSLRLSCVASGFIFSDNYMYWVRQTPEKRLEWVATISDGGSYTYYPDSVKGRFTISRDNAKNNLYLQMSSLKSEDTAIYYCARGYYRYEGAMDYWGQGTSVTVSSGGGGSGGGGSGGGGSNIVMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSWTFGGGTKLEIKTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 487 aa, synthetic construct
MALPVTALLLPLALLLHAARPQVQLVEAGGGVVQPGRSLRLSCAASGFIFSGYGMHWVRQAPGKGLEWVTVIWYDGSNKYYADSVKGRFTISRDNSKNTLYLQMNSLRAEDTAVYYCARDTGDRFFDYWGQGTLVTVSSGGGGSGGGGSGGGGSEIVLTQSPATLSLSPGERATLSCRASQSVSSYLAWYQQKPGQAPRLLIYDASSRATGIPARFGGSGSGTDFTLTISSLEPEDFAVYYCQLRSNWPPYTFGQGTKLEIKTSGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 492 aa, synthetic construct
MALPVTALLLPLALLLHAARPQVQLVESGGGWQPGRSLRLSCSSSGFIFSDNYMYWVRQAPGKGLEWVATISDGGSYTYYPDSVKGRFTISRDNSKNTLFLQMDSLRPEDTGVYFCARGYYRYEGAMDYWGQGTPVTVSSGGGGSGGGGSGGGGSDIQLTQSPSSLSASVGDRVTITCKSSQSVLYSSNQKNYLAWYQQKPGKAPKLLIYWASTRESGVPSRFSGSGSGTDFTFTISSLQPEDIATYYCHQYLSSWTFGQGTKLEIKTSGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 493 aa, synthetic construct
MALPVTALLLPLALLLHAARPEVQLVESGGGLVKPGGSLRLSCVASGFIFSDNYMYWVRQTPEKRLEWVATISDGGSYTYYPDSVKGRFTISRDNAKNNLYLQMSSLKSEDTAIYYCARGYYRYEGAMDYWGQGTSVTVSSGGGGSGGGGSGGGGSNIVMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSWTFGGGTKLEIKTSGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - DNA, 1467 bp, synthetic construct
  source 1..1467: mol_type = other DNA
atggctttgcctgttacagctctgctgctgcccctggctctgcttctgcatgctgctagacctcaggtgcagctggttgaagctggtggcggagttgtgcagcctggcagatctctgagactgagctgtgccgccagcggcttcatctttagcggctatggcatgcactgggtccgacaggcacctggcaaaggcctggaatgggtcaccgtgatttggtacgacggcagcaacaagtactacgccgacagcgtgaagggcagattcaccatcagccgggacaacagcaagaacaccctgtacctgcagatgaacagcctgagagccgaggacaccgccgtgtactactgtgccagagacaccggcgacagattcttcgactattggggccagggcaccctcgtgacagtttcttctggcggcggaggatctggcggaggtggaagcggaggcggtggatctgaaatcgtgctgacacagagccccgccacactgtcactttctccaggcgaaagagccacactgagctgcagagcctctcagagcgtgtcctcttacctggcctggtatcagcagaagcctggacaggctccccggctgctgatctacgatgcttctagcagagctacaggcatccccgccagatttggcggctctggctctggcactgatttcaccctgaccataagcagcctggaacctgaggactttgccgtgtattattgccagctgcggagcaactggcctccttacacatttggccaggggaccaagctggaaatcaagactagtaccaccacaccagctcctcggccacctactccagctccaacaattgccagccagcctctgtctctgaggcccgaagcttgtagacctgctgctggcggagccgtgcatacaagaggactggatttcgcctgcgacatctacatctgggcccctctggctggaacatgtggcgtgctgctgctcagcctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgtctagagtgaagttcagcagaagcgccgacgctcctgcctatcagcagggacagaatcagctgtacaacgagctgaacctggggcgcagagaagagtacgacgtgctggacaagagaagaggcagggaccctgagatgggcggcaagcccagaagaaagaaccctcaagagggcctgtataatgagctgcagaaagacaagatggccgaggcctacagcgagatcggaatgaagggcgaacgcagaagaggaaagggccacgacggactgtatcagggcctgagcacagccaccaaggacacctatgatgccctgcacatgcaggcactgcctccaagatga

SEQ ID NO: 18 - DNA, 1464 bp, synthetic construct
  source 1..1464: mol_type = other DNA
atggctttgcctgttacagctctgctgctgcccctggctctgcttctgcatgctgctagacctcaggtgcagctggttgaagctggtggcggagttgtgcagcctggcagatctctgagactgagctgtgccgccagcggcttcatctttagcggctatggcatgcactgggtccgacaggcacctggcaaaggcctggaatgggtcaccgtgatttggtacgacggcagcaacaagtactacgccgacagcgtgaagggcagattcaccatcagccgggacaacagcaagaacaccctgtacctgcagatgaacagcctgagagccgaggacaccgccgtgtactactgtgccagagacaccggcgacagattcttcgactattggggccagggcaccctcgtgacagtttcttctggcggcggaggatctggcggaggtggaagcggaggcggtggatctgaaatcgtgctgacacagagccccgccacactgtcactttctccaggcgaaagagccacactgagctgcagagcctctcagagcgtgtcctcttacctggcctggtatcagcagaagcctggacaggctccccggctgctgatctacgatgcttctagcagagctacaggcatccccgccagatttggcggctctggctctggcactgatttcaccctgaccataagcagcctggaacctgaggactttgccgtgtattattgccagctgcggagcaactggcctccttacacatttggccaggggaccaagctggaaatcaagactagtggcgccgctgctattgaagtgatgtaccctcctccttacctggacaacgagaagtccaacggcaccatcatccacgtgaagggcaagcacctgtgtccttctccactgttccccggacctagcaagcctttctgggtgctcgttgttgttggcggcgtgctggcctgttattccctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctaccggtctagagtgaagttcagcagatccgccgacgctcctgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagatga

SEQ ID NO: 19 - DNA, 1482 bp, synthetic construct
  source 1..1482: mol_type = other DNA
atggctttgcctgttacagctctgctgctgcccctggctctgcttctgcatgctgctagacctcaggtgcagctggtggaatctggcggaggatggcagcctggcagaagcctgagactgagctgtagcagcagcggcttcatcttcagcgacaactacatgtactgggtccgacaggcccctggcaaaggccttgaatgggtcgccacaatctctgacggcggcagctacacctactatcccgactctgtgaagggcagattcaccatcagccgggacaacagcaagaataccctgttcctgcagatggacagcctgcggcctgaagataccggcgtgtacttttgcgccagaggctactacagatacgagggcgccatggactattggggccagggaacacctgtgaccgtttctagcggcggaggtggaagcggaggcggaggtagtggtggtggcggatctgacattcagctgacacagagccctagcagcctgtctgcctctgtgggcgacagagtgaccatcacatgcaagagcagccagagcgtgctgtactccagcaaccagaagaactacctggcctggtatcagcagaagcccggcaaggctcctaagctgctgatctactgggccagcaccagagaaagcggcgtgccaagcagattttctggcagcggctctggcaccgacttcaccttcaccataagctccctgcagcctgaggacattgccacctactactgccaccagtacctgagcagctggacctttggccagggcaccaagctggaaatcaagactagtaccaccacaccagctcctcggccacctactccagctccaacaattgccagccagcctctgtctctgaggcccgaagcttgtagacctgctgctggcggagccgtgcatacaagaggactggatttcgcctgcgacatctacatctgggcccctctggctggaacatgtggcgtgctgctgctcagcctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgtctagagtgaagttcagcagaagcgccgacgctcctgcctatcagcagggacagaatcagctgtacaacgagctgaacctggggcgcagagaagagtacgacgtgctggacaagagaagaggcagggaccctgagatgggcggcaagcccagaagaaagaaccctcaagagggcctgtataatgagctgcagaaagacaagatggccgaggcctacagcgagatcggaatgaagggcgaacgcagaagaggaaagggccacgacggactgtatcagggcctgagcacagccaccaaggacacctatgatgccctgcacatgcaggcactgcctccaagatga

SEQ ID NO: 20 - DNA, 1479 bp, synthetic construct
  source 1..1479: mol_type = other DNA
atggctttgcctgttacagctctgctgctgcccctggctctgcttctgcatgctgctagacctcaggtgcagctggtggaatctggcggaggatggcagcctggcagaagcctgagactgagctgtagcagcagcggcttcatcttcagcgacaactacatgtactgggtccgacaggcccctggcaaaggccttgaatgggtcgccacaatctctgacggcggcagctacacctactatcccgactctgtgaagggcagattcaccatcagccgggacaacagcaagaataccctgttcctgcagatggacagcctgcggcctgaagataccggcgtgtacttttgcgccagaggctactacagatacgagggcgccatggactattggggccagggaacacctgtgaccgtttctagcggcggaggtggaagcggaggcggaggtagtggtggtggcggatctgacattcagctgacacagagccctagcagcctgtctgcctctgtgggcgacagagtgaccatcacatgcaagagcagccagagcgtgctgtactccagcaaccagaagaactacctggcctggtatcagcagaagcccggcaaggctcctaagctgctgatctactgggccagcaccagagaaagcggcgtgccaagcagattttctggcagcggctctggcaccgacttcaccttcaccataagctccctgcagcctgaggacattgccacctactactgccaccagtacctgagcagctggacctttggccagggcaccaagctggaaatcaagactagtggcgccgctgctattgaagtgatgtaccctcctccttacctggacaacgagaagtccaacggcaccatcatccacgtgaagggcaagcacctgtgtccttctccactgttccccggacctagcaagcctttctgggtgctcgttgttgttggcggcgtgctggcctgttattccctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctaccggtctagagtgaagttcagcagatccgccgacgctcctgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagatga

SEQ ID NO: 21 - DNA, 1485 bp, synthetic construct
  source 1..1485: mol_type = other DNA
atggctctgcctgttacagctctgctgctgcctctggctctgcttctgcatgccgctagacctgaggtgcagctggttgaatctggcggcggacttgtgaagcctggcggatctctgagactgagctgtgtggccagcggcttcatcttcagcgacaactacatgtactgggtccgacagacccctgagaagcggctggaatgggtcgccacaatctctgatggcggcagctacacctactatcccgacagcgtgaagggcagattcaccatcagccgggacaacgccaagaacaacctgtacctgcagatgagcagcctgaagtccgaggacaccgccatctactactgcgccagaggctactacagatacgagggcgccatggactattggggccagggaacaagcgtgacagtgtctagcggaggcggaggatcaggtggcggtggatctggcggtggcggctctaatatcgtgatgacacagagccctagcagcctggctgtgtctgccggcgagaaagtgaccatgagctgcaagagcagccagagcgtgctgtactccagcaaccagaagaactacctggcctggtatcagcagaagcccggacagtctcccaagctgctgatctactgggccagcaccagagaaagcggcgtgcccgatagattcacaggcagcggctctggcaccgacttcaccctgacaatcagctctgtgcaggccgaggatctggccgtgtactactgtcaccagtacctgagcagctggacctttggcggaggcaccaagctggaaatcaagactagtaccaccacaccagctcctcggccacctactccagctccaacaattgccagccagcctctgtctctgaggcccgaagcttgtagacctgctgctggcggagccgtgcatacaagaggactggatttcgcctgcgacatctacatctgggcccctctggctggaacatgtggcgtgctgctgctcagcctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgtctagagtgaagttcagcagaagcgccgacgctcctgcctatcagcagggacagaatcagctgtacaacgagctgaacctggggcgcagagaagagtacgacgtgctggacaagagaagaggcagggaccctgagatgggcggcaagcccagaagaaagaaccctcaagagggcctgtataatgagctgcagaaagacaagatggccgaggcctacagcgagatcggaatgaagggcgaacgcagaagaggaaagggccacgacggactgtatcagggcctgagcacagccaccaaggacacctatgatgccctgcacatgcaggcactgcctccaagatga

SEQ ID NO: 22 - DNA, 1482 bp, synthetic construct
  source 1..1482: mol_type = other DNA
atggctctgcctgttacagctctgctgctgcctctggctctgcttctgcatgccgctagacctgaggtgcagctggttgaatctggcggcggacttgtgaagcctggcggatctctgagactgagctgtgtggccagcggcttcatcttcagcgacaactacatgtactgggtccgacagacccctgagaagcggctggaatgggtcgccacaatctctgatggcggcagctacacctactatcccgacagcgtgaagggcagattcaccatcagccgggacaacgccaagaacaacctgtacctgcagatgagcagcctgaagtccgaggacaccgccatctactactgcgccagaggctactacagatacgagggcgccatggactattggggccagggaacaagcgtgacagtgtctagcggaggcggaggatcaggtggcggtggatctggcggtggcggctctaatatcgtgatgacacagagccctagcagcctggctgtgtctgccggcgagaaagtgaccatgagctgcaagagcagccagagcgtgctgtactccagcaaccagaagaactacctggcctggtatcagcagaagcccggacagtctcccaagctgctgatctactgggccagcaccagagaaagcggcgtgcccgatagattcacaggcagcggctctggcaccgacttcaccctgacaatcagctctgtgcaggccgaggatctggccgtgtactactgtcaccagtacctgagcagctggacctttggcggaggcaccaagctggaaatcaagactagtggcgccgctgctattgaagtgatgtaccctcctccttacctggacaacgagaagtccaacggcaccatcatccacgtgaagggcaagcacctgtgtccttctccactgttccccggacctagcaagcctttctgggtgctcgttgttgttggcggcgtgctggcctgttattccctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctaccggtctagagtgaagttcagcagatccgccgacgctcctgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagatga

SEQ ID NO: 23 - protein, 21 aa, Homo sapiens
MALPVTALLLPLALLLHAARP

SEQ ID NO: 24 - protein, 9 aa, synthetic construct
SGFIFSGYG

SEQ ID NO: 25 - protein, 8 aa, synthetic construct
IWYDGSNK

SEQ ID NO: 26 - protein, 11 aa, synthetic construct
ARDTGDRFFDY

SEQ ID NO: 27 - protein, 24 aa, synthetic construct
QVQLVEAGGGVVQPGRSLRLSCAA

SEQ ID NO: 28 - protein, 17 aa, synthetic construct
MHWVRQAPGKGLEWVTV

SEQ ID NO: 29 - protein, 38 aa, synthetic construct
YYADSVKGRFTISRDNSKNTLYLQMNSLRAEDTAVYYC

SEQ ID NO: 30 - protein, 11 aa, synthetic construct
WGQGTTVTVSS

SEQ ID NO: 31 - protein, 118 aa, synthetic construct
QVQLVEAGGGVVQPGRSLRLSCAASGFIFSGYGMHWVRQAPGKGLEWVTVIWYDGSNKYYADSVKGRFTISRDNSKNTLYLQMNSLRAEDTAVYYCARDTGDRFFDYWGQGTLVTVSS

SEQ ID NO: 32 - protein, 6 aa, synthetic construct
QSVSSY

SEQ ID NO: 33 - skipped

SEQ ID NO: 34 - protein, 10 aa, synthetic construct
QLRSNWPPYT

SEQ ID NO: 35 - protein, 26 aa, synthetic construct [sequence omitted]

SEQ ID NO: 36 - protein, 17 aa, synthetic construct
LAWYQQKPGQAPRLLIY

SEQ ID NO: 37 - protein, 36 aa, synthetic construct
SRATGIPARFGGSGSGTDFTLTISSLEPEDFAVYYC

SEQ ID NO: 38 - protein, 12 aa, synthetic construct
FGQGTKLEIKTS

SEQ ID NO: 39 - protein, 110 aa, synthetic construct
EIVLTQSPATLSLSPGERATLSCRASQSVSSYLAWYQQKPGQAPRLLIYDASSRATGIPARFGGSGSGTDFTLTISSLEPEDFAVYYCQLRSNWPPYTFGQGTKLEIKTS

SEQ ID NO: 40 - protein, 5 aa, synthetic construct
GYGMH

SEQ ID NO: 41 - protein, 17 aa, synthetic construct
VIWYDGSNKYYADSVKG

SEQ ID NO: 42 - protein, 9 aa, synthetic construct
DTGDRFFDY

SEQ ID NO: 43 - protein, 30 aa, synthetic construct
QVQLVEAGGGVVQPGRSLRLSCAASGFIFS

SEQ ID NO: 44 - protein, 14 aa, synthetic construct
WVRQAPGKGLEWVT

SEQ ID NO: 45 - protein, 31 aa, synthetic construct
FTISRDNSKNTLYLQMNSLRAEDTAVYYCAR

SEQ ID NO: 46 - protein, 11 aa, synthetic construct
WGQGTLVTVSS

SEQ ID NO: 47 - protein, 118 aa, synthetic construct; identical to SEQ ID NO: 31

SEQ ID NO: 48 - protein, 11 aa, synthetic construct
RASQSVSSYLA

SEQ ID NO: 49 - protein, 7 aa, synthetic construct
DASSRAT

SEQ ID NO: 50 - protein, 10 aa, synthetic construct
QLRSNWPPYT

SEQ ID NO: 51 - protein, 23 aa, synthetic construct
EIVLTQSPATLSLSPGERATLSC

SEQ ID NO: 52 - protein, 15 aa, synthetic construct
WYQQKPGQAPRLLIY

SEQ ID NO: 53 - protein, 32 aa, synthetic construct
GIPARFGGSGSGTDFTLTISSLEPEDFAVYYC

SEQ ID NO: 54 - protein, 12 aa, synthetic construct
FGQGTKLEIKTS

SEQ ID NO: 55 - protein, 110 aa, synthetic construct; identical to SEQ ID NO: 39

SEQ ID NO: 56 - protein, 9 aa, synthetic construct
TGFIFSGYG

SEQ ID NO: 57 - protein, 9 aa, synthetic construct
CGFIFSGYG

SEQ ID NO: 58 - protein, 9 aa, synthetic construct
GGFIFSGYG

SEQ ID NO: 59 - protein, 9 aa, synthetic construct
SSFIFSGYG

SEQ ID NO: 60 - protein, 9 aa, synthetic construct
SCFIFSGYG

SEQ ID NO: 61 - protein, 9 aa, synthetic construct
SQFIFSGYG

SEQ ID NO: 62 - protein, 9 aa, synthetic construct
SNFIFSGYG

SEQ ID NO: 63 - protein, 9 aa, synthetic construct
STFIFSGYG

SEQ ID NO: 64 - protein, 9 aa, synthetic construct
SGWIFSGYG

SEQ ID NO: 65 - protein, 9 aa, synthetic construct
SGFIWSGYG

SEQ ID NO: 66 - protein, 9 aa, synthetic construct
SGWIWSGYG

SEQ ID NO: 67 - protein, 9 aa, synthetic construct
SGPIFSGYG

SEQ ID NO: 68 - protein, 9 aa, synthetic construct
SGFIPSGYG

SEQ ID NO: 69 - protein, 9 aa, synthetic construct
SGPIPSGYG

SEQ ID NO: 70 - protein, 9 aa, synthetic construct
SGYIFSGYG

SEQ ID NO: 71 - protein, 9 aa, synthetic construct
SGFIYSGYG

SEQ ID NO: 72 - protein, 9 aa, synthetic construct
SGYIYSGYG

SEQ ID NO: 73 - protein, 9 aa, synthetic construct
SGFLFSGYG

SEQ ID NO: 74 - protein, 9 aa, synthetic construct
SGFVFSGYG

SEQ ID NO: 75 - protein, 9 aa, synthetic construct
SGFAFSGYG

SEQ ID NO: 76 - protein, 9 aa, synthetic construct
SGFMFSGYG

SEQ ID NO: 77 - protein, 9 aa, synthetic construct
SGFIFTGYG

SEQ ID NO: 78 - protein, 9 aa, synthetic construct
SGFIFCGYG

SEQ ID NO: 79 - protein, 9 aa, synthetic construct
SGFIFGGYG

SEQ ID NO: 80 - protein, 9 aa, synthetic construct
SGFIFSSYG

SEQ ID NO: 81 - protein, 9 aa, synthetic construct
SGFIFSCYG

SEQ ID NO: 82 - protein, 9 aa, synthetic construct
SGFIFSTYG

SEQ ID NO: 83 - protein, 9 aa, synthetic construct
SGFIFSQYG

SEQ ID NO: 84 - protein, 9 aa, synthetic construct
SGFIFSNYG

SEQ ID NO: 85 - protein, 9 aa, synthetic construct
SGFIFSGYS

SEQ ID NO: 86 - protein, 9 aa, synthetic construct
SGFIFSGYC

SEQ ID NO: 87 - protein, 9 aa, synthetic construct
SGFIFSGYT

SEQ ID NO: 88 - protein, 9 aa, synthetic construct
SGFIFSGYQ

SEQ ID NO: 89 - protein, 9 aa, synthetic construct
SGFIFSGYN

SEQ ID NO: 90 - protein, 9 aa, synthetic construct
SGFIFSGFG

SEQ ID NO: 91 - protein, 9 aa, synthetic construct
SGFIFSGSG

SEQ ID NO: 92 - protein, 9 aa, synthetic construct
SGFIFSGTG

SEQ ID NO: 93 - protein, 8 aa, synthetic construct
LWYDGSNK

SEQ ID NO: 94 - protein, 8 aa, synthetic construct
VWYDGSNK

SEQ ID NO: 95 - protein, 8 aa, synthetic construct
AWYDGSNK

SEQ ID NO: 96 - protein, 8 aa, synthetic construct
IFYDGSNK

SEQ ID NO: 97 - protein, 8 aa, synthetic construct
IPYDGSNK

SEQ ID NO: 98 - protein, 8 aa, synthetic construct
IHYDGSNK

SEQ ID NO: 99 - protein, 8 aa, synthetic construct
IWFDGSNK

SEQ ID NO: 100 - protein, 8 aa, synthetic construct
IWSDGSNK

SEQ ID NO: 101 - protein, 8 aa, synthetic construct
IWTDGSNK

SEQ ID NO: 102 - protein, 8 aa, synthetic construct
IWYEGSNK

SEQ ID NO: 103 - protein, 8 aa, synthetic construct
IWYNGSNK

SEQ ID NO: 104 - protein, 8 aa, synthetic construct
IWYDSSNK

SEQ ID NO: 105 - protein, 8 aa, synthetic construct
IWYDTSNK

SEQ ID NO: 106 - protein, 8 aa, synthetic construct
IWYDCSNK

SEQ ID NO: 107 - protein, 8 aa, synthetic construct
IWYDNSNK

SEQ ID NO: 108 - protein, 8 aa, synthetic construct
IWYDQSNK

SEQ ID NO: 109 - protein, 8 aa, synthetic construct
IWYDGCNK

SEQ ID NO: 110 - protein, 8 aa, synthetic construct
IWYDGTNK

SEQ ID NO: 111 - protein, 8 aa, synthetic construct
IWYDGGNK

SEQ ID NO: 112 - protein, 8 aa, synthetic construct
IWYDGNNK

SEQ ID NO: 113 - protein, 8 aa, synthetic construct
IWYDGQNK

SEQ ID NO: 114 - protein, 8 aa, synthetic construct
IWYDGSQK

SEQ ID NO: 115 - protein, 8 aa, synthetic construct
IWYDGSGK

SEQ ID NO: 116 - protein, 8 aa, synthetic construct
IWYDGSSK

SEQ ID NO: 117 - protein, 8 aa, synthetic construct
IWYDGSCK

SEQ ID NO: 118 - protein, 8 aa, synthetic construct
IWYDGSTK

SEQ ID NO: 119 - protein, 8 aa, synthetic construct
IWYDGSNR

SEQ ID NO: 120 - protein, 8 aa, synthetic construct
IWYDGSNH

SEQ ID NO: 121 - protein, 11 aa, synthetic construct
VRDTGDRFFDY

SEQ ID NO: 122 - protein, 11 aa, synthetic construct
LRDTGDRFFDY

SEQ ID NO: 123 - protein, 11 aa, synthetic construct
IRDTGDRFFDY

SEQ ID NO: 124 - protein, 11 aa, synthetic construct
AKDTGDRFFDY

SEQ ID NO: 125 - protein, 11 aa, synthetic construct
AHDTGDRFFDY

SEQ ID NO: 126 - protein, 11 aa, synthetic construct
ARETGDRFFDY

SEQ ID NO: 127 - protein, 11 aa, synthetic construct
ARDSGDRFFDY

SEQ ID NO: 128 - protein, 11 aa, synthetic construct
ARDCGDRFFDY

SEQ ID NO: 129 - protein, 11 aa, synthetic construct
ARDYGDRFFDY

SEQ ID NO: 130 - protein, 11 aa, synthetic construct
ARDTQDRFFDY

SEQ ID NO: 131 - protein, 11 aa, synthetic construct
ARDTCDRFFDY

SEQ ID NO: 132 - protein, 11 aa, synthetic construct
ARDTTDRFFDY

SEQ ID NO: 133 - protein, 11 aa, synthetic construct
ARDTSDRFFDY

SEQ ID NO: 134 - protein, 11 aa, synthetic construct
ARDTGERFFDY

SEQ ID NO: 135 - protein, 11 aa, synthetic construct
ARDTGDKFFDY

SEQ ID NO: 136 - protein, 11 aa, synthetic construct
ARDTGDHFFDY

SEQ ID NO: 137 - protein, 11 aa, synthetic construct
ARDTGDRWFDY

SEQ ID NO: 138 - protein, 11 aa, synthetic construct
ARDTGDRPFDY

SEQ ID NO: 139 - protein, 11 aa, synthetic construct
ARDTGDRYFDY

SEQ ID NO: 140 - protein, 11 aa, synthetic construct
ARDTGDRFWDY

SEQ ID NO: 141 - protein, 11 aa, synthetic construct
ARDTGDRFPDY

SEQ ID NO: 142 - protein, 11 aa, synthetic construct
ARDTGDRFYDY

SEQ ID NO: 143 - protein, 11 aa, synthetic construct
ARDTGDRFFEY

SEQ ID NO: 144 - protein, 11 aa, synthetic construct
ARDTGDRFFDT

SEQ ID NO: 145 - protein, 11 aa, synthetic construct
ARDTGDRFFDS

SEQ ID NO: 146 - protein, 11 aa, synthetic construct
ARDTGDRFFDF

SEQ ID NO: 147 - protein, 6 aa, synthetic construct
GSVSSY

SEQ ID NO: 148 - protein, 6 aa, synthetic construct
NSVSSY

SEQ ID NO: 149 - protein, 6 aa, synthetic construct
QTVSSY

SEQ ID NO: 150 - protein, 6 aa, synthetic construct
QCVSSY

SEQ ID NO: 151 - protein, 6 aa, synthetic construct
QYVSSY

SEQ ID NO: 152 - protein, 6 aa, synthetic construct
QSASSY

SEQ ID NO: 153 - protein, 6 aa, synthetic construct
QSLSSY

SEQ ID NO: 154 - protein, 6 aa, synthetic construct
QSISSY

SEQ ID NO: 155 - protein, 6 aa, synthetic construct
QSMSSY

SEQ ID NO: 156 - protein, 6 aa, synthetic construct
QSVTSY

SEQ ID NO: 157 - protein, 6 aa, synthetic construct
QSVCSY

SEQ ID NO: 158 - protein, 6 aa, synthetic construct
QSVYSY

SEQ ID NO: 159 - protein, 6 aa, synthetic construct
QSVSTY

SEQ ID NO: 160 - protein, 6 aa, synthetic construct
QSVSCY

SEQ ID NO: 161 - protein, 6 aa, synthetic construct
QSVSYY

SEQ ID NO: 162 - protein, 6 aa, synthetic construct
QSVSSS

SEQ ID NO: 163 - protein, 6 aa, synthetic construct
QSVSST

SEQ ID NO: 164 - protein, 6 aa, synthetic construct
QSVSSF

SEQ ID NO: 165-172 - skipped (8 entries)

SEQ ID NO: 173 - protein, 10 aa, synthetic construct
NLRSNWPPYT

SEQ ID NO: 174 - protein, 10 aa, synthetic construct
PLRSNWPPYT

SEQ ID NO: 175 - protein, 10 aa, synthetic construct
QIRSNWPPYT

SEQ ID NO: 176 - protein, 10 aa, synthetic construct
QVRSNWPPYT

SEQ ID NO: 177 - protein, 10 aa, synthetic construct
QARSNWPPYT

SEQ ID NO: 178 - protein, 10 aa, synthetic construct
QLKSNWPPYT

SEQ ID NO: 179 - protein, 10 aa, synthetic construct
QLHSNWPPYT

SEQ ID NO: 180 - protein, 10 aa, synthetic construct
QLRTNWPPYT

SEQ ID NO: 181 - protein, 10 aa, synthetic construct
QLRCNWPPYT

SEQ ID NO: 182 - protein, 10 aa, synthetic construct
QLRYNWPPYT

SEQ ID NO: 183 - protein, 10 aa, synthetic construct
QLRSQWPPYT

SEQ ID NO: 184 - protein, 10 aa, synthetic construct
QLRSNFPPYT

SEQ ID NO: 185 - protein, 10 aa, synthetic construct
QLRSNYPPYT

SEQ ID NO: 186 - protein, 10 aa, synthetic construct
QLRSNPPPYT

SEQ ID NO: 187 - protein, 10 aa, synthetic construct
QLRSNWFPYT

SEQ ID NO: 188 - protein, 10 aa, synthetic construct
QLRSNWQPYT

SEQ ID NO: 189 - protein, 10 aa, synthetic construct
QLRSNWNPYT

SEQ ID NO: 190 - protein, 10 aa, synthetic construct
QLRSNWPWYT

SEQ ID NO: 191 - protein, 10 aa, synthetic construct
QLRSNWWPYT

SEQ ID NO: 192 - protein, 10 aa, synthetic construct
QLRSNWPNYT

SEQ ID NO: 193 - protein, 10 aa, synthetic construct
QLRSNWPQYT

SEQ ID NO: 194 - protein, 10 aa, synthetic construct
QLRSNWPPTT

SEQ ID NO: 195 - protein, 10 aa, synthetic construct
QLRSNWPPST

SEQ ID NO: 196 - protein, 10 aa, synthetic construct
QLRSNWPPFT

SEQ ID NO: 197 - protein, 10 aa, synthetic construct
QLRSNWPPYS

SEQ ID NO: 198 - protein, 10 aa, synthetic construct
QLRSNWPPYC

SEQ ID NO: 199 - protein, 10 aa, synthetic construct
QLRSNWPPYY

SEQ ID NO: 200 - protein, 5 aa, synthetic construct
SYGMH

SEQ ID NO: 201 - protein, 5 aa, synthetic construct
CYGMH

SEQ ID NO: 202 - protein, 5 aa, synthetic construct
TYGMH

SEQ ID NO: 203 - protein, 5 aa, synthetic construct
QYGMH

SEQ ID NO: 204 - protein, 5 aa, synthetic construct
NYGMH

SEQ ID NO: 205 - protein, 5 aa, synthetic construct
GYSMH

SEQ ID NO: 206 - protein, 5 aa, synthetic construct
GYCMH

SEQ ID NO: 207 - protein, 5 aa, synthetic construct
GYTMH

SEQ ID NO: 208 - protein, 5 aa, synthetic construct
GYQMH

SEQ ID NO: 209 - protein, 5 aa, synthetic construct
GYNMH

SEQ ID NO: 210 - protein, 5 aa, synthetic construct
GFGMH

SEQ ID NO: 211 - protein, 5 aa, synthetic construct
GSGMH

SEQ ID NO: 212 - protein, 5 aa, synthetic construct
GTGMH

SEQ ID NO: 213 - protein, 5 aa, synthetic construct
GYGIH

SEQ ID NO: 214 - protein, 5 aa, synthetic construct
GYGLH

SEQ ID NO: 215 - protein, 5 aa, synthetic construct
GYGVH

SEQ ID NO: 216 - protein, 5 aa, synthetic construct
GYGAH

SEQ ID NO: 217 - protein, 5 aa, synthetic construct
GYGCH

SEQ ID NO: 218 - protein, 5 aa, synthetic construct
GYGMK

SEQ ID NO: 219 - protein, 5 aa, synthetic construct
GYGMR

SEQ ID NO: 220 - protein, 5 aa, synthetic construct
GYGMP

SEQ ID NO: 221 - protein, 17 aa, synthetic construct
LIWYDGSNKYYADSVKG

SEQ ID NO: 222 - protein, 17 aa, synthetic construct
IIWYDGSNKYYADSVKG

SEQ ID NO: 223 - protein, 17 aa, synthetic construct
AIWYDGSNKYYADSVKG

SEQ ID NO: 224 - protein, 17 aa, synthetic construct
MIWYDGSNKYYADSVKG

SEQ ID NO: 225 - protein, 17 aa, synthetic construct
VLWYDGSNKYYADSVKG

SEQ ID NO: 226 - protein, 17 aa, synthetic construct
VVWYDGSNKYYADSVKG

SEQ ID NO: 227 - protein, 17 aa, synthetic construct
VAWYDGSNKYYADSVKG

SEQ ID NO: 228 - protein, 17 aa, synthetic construct
VMWYDGSNKYYADSVKG

SEQ ID NO: 229 - protein, 18 aa, synthetic construct
VILWYDGSNKYYADSVKG

SEQ ID NO: 230 - protein, 18 aa, synthetic construct
VIVWYDGSNKYYADSVKG

SEQ ID NO: 231 - protein, 18 aa, synthetic construct
VIAWYDGSNKYYADSVKG

SEQ ID NO: 232 - protein, 18 aa, synthetic construct
VIIFYDGSNKYYADSVKG

SEQ ID NO: 233 - protein, 18 aa, synthetic construct
VIIPYDGSNKYYADSVKG

SEQ ID NO: 234 - protein, 18 aa, synthetic construct
VIIHYDGSNKYYADSVKG

SEQ ID NO: 235 - protein, 18 aa, synthetic construct
VIIWFDGSNKYYADSVKG

SEQ ID NO: 236 - protein, 18 aa, synthetic construct
VIIWSDGSNKYYADSVKG

SEQ ID NO: 237 - protein, 18 aa, synthetic construct
VIIWTDGSNKYYADSVKG

SEQ ID NO: 238 - protein, 18 aa, synthetic construct
VIIWYEGSNKYYADSVKG

SEQ ID NO: 239 - protein, 18 aa, synthetic construct
VIIWYNGSNKYYADSVKG

SEQ ID NO: 240 - protein, 18 aa, synthetic construct
VIIWYDSSNKYYADSVKG

SEQ ID NO: 241 - protein, 18 aa, synthetic construct
VIIWYDTSNKYYADSVKG

SEQ ID NO: 242 - protein, 18 aa, synthetic construct
VIIWYDCSNKYYADSVKG

SEQ ID NO: 243 - protein, 18 aa, synthetic construct
VIIWYDNSNKYYADSVKG

SEQ ID NO: 244 - protein, 18 aa, synthetic construct
VIIWYDQSNKYYADSVKG

SEQ ID NO: 245 - protein, 18 aa, synthetic construct
VIIWYDGCNKYYADSVKG

SEQ ID NO: 246 - protein, 18 aa, synthetic construct
VIIWYDGTNKYYADSVKG

SEQ ID NO: 247 - protein, 18 aa, synthetic construct
VIIWYDGGNKYYADSVKG

SEQ ID NO: 248 - protein, 18 aa, synthetic construct
VIIWYDGNNKYYADSVKG

SEQ ID NO: 249 - protein, 18 aa, synthetic construct
VIIWYDGQNKYYADSVKG

SEQ ID NO: 250 - protein, 18 aa, synthetic construct
VIIWYDGSQKYYADSVKG

SEQ ID NO: 251 - protein, 18 aa, synthetic construct
VIIWYDGSGKYYADSVKG

SEQ ID NO: 252 - protein, 18 aa, synthetic construct
VIIWYDGSSKYYADSVKG

SEQ ID NO: 253 - protein, 18 aa, synthetic construct
VIIWYDGSCKYYADSVKG

SEQ ID NO: 254 - protein, 18 aa, synthetic construct
VIIWYDGSTKYYADSVKG

SEQ ID NO: 255 - protein, 18 aa, synthetic construct
VIIWYDGSNRYYADSVKG

SEQ ID NO: 256 - protein, 18 aa, synthetic construct
VIIWYDGSNHYYADSVKG

SEQ ID NO: 257 - protein, 17 aa, synthetic construct
VIWYDGSNKSYADSVKG

SEQ ID NO: 258 - protein, 17 aa, synthetic construct
VIWYDGSNKTYADSVKG

SEQ ID NO: 259 - protein, 17 aa, synthetic construct
VIWYDGSNKFYADSVKG

SEQ ID NO: 260 - protein, 17 aa, synthetic construct
VIWYDGSNKYSADSVKG

SEQ ID NO: 261 - protein, 17 aa, synthetic construct
VIWYDGSNKYTADSVKG

SEQ ID NO: 262 - protein, 17 aa, synthetic construct
VIWYDGSNKYFADSVKG

SEQ ID NO: 263 - protein, 17 aa, synthetic construct
VIWYDGSNKYYVDSVKG

SEQ ID NO: 264 - protein, 17 aa, synthetic construct
VIWYDGSNKYYLDSVKG

SEQ ID NO: 265 - protein, 17 aa, synthetic construct
VIWYDGSNKYYIDSVKG

SEQ ID NO: 266 - protein, 17 aa, synthetic construct
VIWYDGSNKYYMDSVKG

SEQ ID NO: 267 - protein, 17 aa, synthetic construct
VIWYDGSNKYYAESVKG

SEQ ID NO: 268 - protein, 17 aa, synthetic construct
VIWYDGSNKYYADCVKG

SEQ ID NO: 269 - protein, 17 aa, synthetic construct
VIWYDGSNKYYADTVKG

SEQ ID NO: 270 - protein, 17 aa, synthetic construct
VIWYDGSNKYYADSAKG

SEQ ID NO: 271 - protein, 17 aa, synthetic construct
VIWYDGSNKYYADSLKG

SEQ ID NO: 272 - protein, 17 aa, synthetic construct
VIWYDGSNKYYADSIKG

SEQ ID NO: 273 - protein, 17 aa, synthetic construct
VIWYDGSNKYYADSMKG

SEQ ID NO: 274 - protein, 17 aa, synthetic construct
VIWYDGSNKYYADSVRG

SEQ ID NO: 275 - protein, 17 aa, synthetic construct
VIWYDGSNKYYADSVHG

SEQ ID NO: 276 - protein, 17 aa, synthetic construct
VIWYDGSNKYYADSVKS

SEQ ID NO: 277 - protein, 17 aa, synthetic construct
VIWYDGSNKYYADSVKT

SEQ ID NO: 278 - protein, 17 aa, synthetic construct
VIWYDGSNKYYADSVKC

SEQ ID NO: 279 - protein, 9 aa, synthetic construct
ETGDRFFDY

SEQ ID NO: 280 - protein, 9 aa, synthetic construct
DSGDRFFDY

SEQ ID NO: 281 - protein, 9 aa, synthetic construct
DCGDRFFDY

SEQ ID NO: 282 - protein, 9 aa, synthetic construct
DYGDRFFDY

SEQ ID NO: 283 - protein, 9 aa, synthetic construct
DTQDRFFDY

SEQ ID NO: 284 - protein, 9 aa, synthetic construct
DTCDRFFDY

SEQ ID NO: 285 - protein, 9 aa, synthetic construct
DTTDRFFDY

SEQ ID NO: 286 - protein, 9 aa, synthetic construct
DTSDRFFDY

SEQ ID NO: 287 - protein, 9 aa, synthetic construct
DTGERFFDY

SEQ ID NO: 288 - protein, 9 aa, synthetic construct
DTGDKFFDY

SEQ ID NO: 289 - protein, 9 aa, synthetic construct
DTGDHFFDY

SEQ ID NO: 290 - protein, 9 aa, synthetic construct
DTGDRWFDY

SEQ ID NO: 291 - protein, 9 aa, synthetic construct
DTGDRPFDY

SEQ ID NO: 292 - protein, 9 aa, synthetic construct
DTGDRYFDY

SEQ ID NO: 293 - protein, 9 aa, synthetic construct
DTGDRFWDY

SEQ ID NO: 294 - protein, 9 aa, synthetic construct
DTGDRFPDY

SEQ ID NO: 295 - protein, 9 aa, synthetic construct
DTGDRFYDY

SEQ ID NO: 296 - protein, 9 aa, synthetic construct
DTGDRFFEY

SEQ ID NO: 297 - protein, 9 aa, synthetic construct
DTGDRFFDT

SEQ ID NO: 298 - protein, 9 aa, synthetic construct
DTGDRFFDS

SEQ ID NO: 299 - protein, 9 aa, synthetic construct
DTGDRFFDF

SEQ ID NO: 300 - protein, 11 aa, synthetic construct
KASQSVSSYLA

SEQ ID NO: 301 - protein, 11 aa, synthetic construct
HASQSVSSYLA

SEQ ID NO: 302 - protein, 11 aa, synthetic construct
RVSQSVSSYLA

SEQ ID NO: 303 - protein, 11 aa, synthetic construct
RLSQSVSSYLA

SEQ ID NO: 304 - protein, 11 aa, synthetic construct
RISQSVSSYLA

SEQ ID NO: 305 - protein, 11 aa, synthetic construct
RMSQSVSSYLA

SEQ ID NO: 306 - protein, 11 aa, synthetic construct
RATQSVSSYLA

SEQ ID NO: 307 - protein, 11 aa, synthetic construct
RACQSVSSYLA

SEQ ID NO: 308 - protein, 11 aa, synthetic construct
RAYQSVSSYLA

SEQ ID NO: 309 - protein, 11 aa, synthetic construct
RASGSVSSYLA

SEQ ID NO: 310 - protein, 11 aa, synthetic construct
RASNSVSSYLA

SEQ ID NO: 311 - protein, 11 aa, synthetic construct
RASQTVSSYLA

SEQ ID NO: 312 - protein, 11 aa, synthetic construct
RASQCVSSYLA

SEQ ID NO: 313 - protein, 11 aa, synthetic construct
RASQYVSSYLA

SEQ ID NO: 314 - protein, 11 aa, synthetic construct
RASQSASSYLA

SEQ ID NO: 315 - protein, 11 aa, synthetic construct
RASQSLSSYLA

SEQ ID NO: 316 - protein, 11 aa, synthetic construct
RASQSISSYLA

SEQ ID NO: 317 - protein, 11 aa, synthetic construct
RASQSMSSYLA

SEQ ID NO: 318 - protein, 11 aa, synthetic construct
RASQSVTSYLA

SEQ ID NO: 319 - protein, 11 aa, synthetic construct
RASQSVCSYLA

SEQ ID NO: 320 - protein, 11 aa, synthetic construct
RASQSVYSYLA

SEQ ID NO: 321 - protein, 11 aa, synthetic construct
RASQSVSTYLA

SEQ ID NO: 322 - protein, 11 aa, synthetic construct
RASQSVSCYLA

SEQ ID NO: 323 - protein, 11 aa, synthetic construct
RASQSVSYYLA

SEQ ID NO: 324 - protein, 11 aa, synthetic construct
RASQSVSSSLA

SEQ ID NO: 325 - protein, 11 aa, synthetic construct
RASQSVSSTLA

SEQ ID NO: 326 - protein, 11 aa, synthetic construct
RASQSVSSFLA

SEQ ID NO: 327 - protein, 11 aa, synthetic construct
RASQSVSSYIA

SEQ ID NO: 328 - protein, 11 aa, synthetic construct
RASQSVSSYVA

SEQ ID NO: 329 - protein, 11 aa, synthetic construct
RASQSVSSYAA

SEQ ID NO: 330 - protein, 11 aa, synthetic construct
RASQSVSSYMA

SEQ ID NO: 331 - protein, 11 aa, synthetic construct
RASQSVSSYLV

SEQ ID NO: 332 - protein, 11 aa, synthetic construct
RASQSVSSYLL

SEQ ID NO: 333 - protein, 11 aa, synthetic construct
RASQSVSSYLI

SEQ ID NO: 334 - protein, 11 aa, synthetic construct
RASQSVSSYLM

SEQ ID NO: 335 - protein, 7 aa, synthetic construct
EASSRAT

SEQ ID NO: 336 - protein, 7 aa, synthetic construct
DVSSRAT

SEQ ID NO: 337 - protein, 7 aa, synthetic construct
DLSSRAT

SEQ ID NO: 338 - protein, 7 aa, synthetic construct
DISSRAT

SEQ ID NO: 339 - protein, 7 aa, synthetic construct
DMSSRAT

SEQ ID NO: 340 - protein, 7 aa, synthetic construct
DATSRAT

SEQ ID NO: 341 - protein, 7 aa, synthetic construct
DACSRAT

SEQ ID NO: 342 - protein, 7 aa, synthetic construct
DAYSRAT

SEQ ID NO: 343 - protein, 7 aa, synthetic construct
DASTRAT

SEQ ID NO: 344 - protein, 7 aa, synthetic construct
DASCRAT

SEQ ID NO: 345 - protein, 7 aa, synthetic construct
DASYRAT

SEQ ID NO: 346 - protein, 7 aa, synthetic construct
DASSKAT

SEQ ID NO: 347 - protein, 7 aa, synthetic construct
DASSHAT

SEQ ID NO: 348 - protein, 7 aa, synthetic construct
DASSRVT

SEQ ID NO: 349 - protein, 7 aa, synthetic construct
DASSRLT

SEQ ID NO: 350 - protein, 7 aa, synthetic construct
DASSRIT

SEQ ID NO: 351 - protein, 7 aa, synthetic construct
DASSRAS

SEQ ID NO: 352 - protein, 7 aa, synthetic construct
DASSRAC

SEQ ID NO: 353 - protein, 7 aa, synthetic construct
DASSRAY

SEQ ID NO: 354 - protein, 10 aa, synthetic construct
NLRSNWPPYT

SEQ ID NO: 355 - protein, 10 aa, synthetic construct
PLRSNWPPYT

SEQ ID NO: 356 - protein, 10 aa, synthetic construct
QIRSNWPPYT

SEQ ID NO: 357 - protein, 10 aa, synthetic construct
QVRSNWPPYT

SEQ ID NO: 358 - protein, 10 aa, synthetic construct
QARSNWPPYT

SEQ ID NO: 359 - protein, 10 aa, synthetic construct
QLKSNWPPYT

SEQ ID NO: 360 - protein, 10 aa, synthetic construct
QLHSNWPPYT

SEQ ID NO: 361 - protein, 10 aa, synthetic construct
QLRTNWPPYT

SEQ ID NO: 362 - protein, 10 aa, synthetic construct
QLRCNWPPYT

SEQ ID NO: 363 - protein, 10 aa, synthetic construct
QLRYNWPPYT

SEQ ID NO: 364 - protein, 10 aa, synthetic construct
QLRSQWPPYT

SEQ ID NO: 365 - protein, 10 aa, synthetic construct
QLRSNFPPYT

SEQ ID NO: 366 - protein, 10 aa, synthetic construct
QLRSNYPPYT

SEQ ID NO: 367 - protein, 10 aa, synthetic construct
QLRSNPPPYT

SEQ ID NO: 368 - protein, 10 aa, synthetic construct
QLRSNWFPYT

SEQ ID NO: 369 - protein, 10 aa, synthetic construct
QLRSNWQPYT

SEQ ID NO: 370 - protein, 10 aa, synthetic construct
QLRSNWNPYT

SEQ ID NO: 371 - protein, 10 aa, synthetic construct
QLRSNWPWYT

SEQ ID NO: 372 - protein, 10 aa, synthetic construct
QLRSNWWPYT

SEQ ID NO: 373 - protein, 10 aa, synthetic construct
QLRSNWPNYT

SEQ ID NO: 374 - protein, 10 aa, synthetic construct
QLRSNWPQYT

SEQ ID NO: 375 - protein, 10 aa, synthetic construct
QLRSNWPPTT

SEQ ID NO: 376 - protein, 10 aa, synthetic construct
QLRSNWPPST

SEQ ID NO: 377 - protein, 10 aa, synthetic construct
QLRSNWPPFT

SEQ ID NO: 378 - protein, 10 aa, synthetic construct
QLRSNWPPYS

SEQ ID NO: 379 - protein, 10 aa, synthetic construct
QLRSNWPPYC

SEQ ID NO: 380 - protein, 10 aa, synthetic construct
QLRSNWPPYY

SEQ ID NO: 381 - protein, 9 aa, synthetic construct
WGQGTLVTV

SEQ ID NO: 382 - protein, 10 aa, synthetic construct
FGQGTKLEIK

SEQ ID NO: 383 - protein, 9 aa, synthetic construct
WGQGTLVTV

SEQ ID NO: 384 - protein, 10 aa, synthetic construct
FGQGTKLEIK

SEQ ID NO: 385 - protein, 120 aa, synthetic construct
EVQLVESGGGLVKPGGSLRLSCVASGFIFSDNYMYWVRQTPEKRLEWVATISDGGSYTYYPDSVKGRFTISRDNAKNNLYLQMSSLKSEDTAIYYCARGYYRYEGAMDYWGQGTSVTVSS

SEQ ID NO: 386 - protein, 25 aa, synthetic construct
EVQLVESGGGLVKPGGSLRLSCVAS

SEQ ID NO: 387 - protein, 8 aa, synthetic construct
GFIFSDNY

SEQ ID NO: 388 - protein, 17 aa, synthetic construct
MYWVRQTPEKRLEWVAT

SEQ ID NO: 389 - protein, 8 aa, synthetic construct
ISDGGSYT

SEQ ID NO: 390 - protein, 38 aa, synthetic construct
YYPDSVKGRFTISRDNAKNNLYLQMSSLKSEDTAIYYC

SEQ ID NO: 391 - protein, 13 aa, synthetic construct
ARGYYRYEGAMDY

SEQ ID NO: 392 - protein, 11 aa, synthetic construct
WGQGTSVTVSS

SEQ ID NO: 393 - protein, 9 aa, synthetic construct
WGQGTSVTV

SEQ ID NO: 394 - protein, 114 aa, synthetic construct
NIVMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSWTFGGGTKLEIKTS

SEQ ID NO: 395 - protein, 26 aa, synthetic construct
NIVMTQSPSSLAVSAGEKVTMSCKSS

SEQ ID NO: 396 - protein, 12 aa, synthetic construct
QSVLYSSNQKNY

SEQ ID NO: 397 - protein, 17 aa, synthetic construct
LAWYQQKPGQSPKLLIY

SEQ ID NO: 398 - skipped

SEQ ID NO: 399 - protein, 36 aa, synthetic construct
TRESGVPDRFTGSGSGTDFTLTISSVQAEDLAVYYC

SEQ ID NO: 400 - protein, 8 aa, synthetic construct
HQYLSSWT

SEQ ID NO: 401 - protein, 12 aa, synthetic construct
FGGGTKLEIKTS

SEQ ID NO: 402 - protein, 10 aa, synthetic construct
FGGGTKLEIK

SEQ ID NO: 403 - protein, 120 aa, synthetic construct; identical to SEQ ID NO: 385

SEQ ID NO: 404 - protein, 30 aa, synthetic construct
EVQLVESGGGLVKPGGSLRLSCVASGFIFS

SEQ ID NO: 405 - protein, 5 aa, synthetic construct
DNYMY

SEQ ID NO: 406 - protein, 14 aa, synthetic construct
WVRQTPEKRLEWVA

SEQ ID NO: 407 - protein, 17 aa, synthetic construct
TISDGGSYTYYPDSVKG

SEQ ID NO: 408 - protein, 32 aa, synthetic construct
RFTISRDNAKNNLYLQMSSLKSEDTAIYYCAR

SEQ ID NO: 409 - protein, 11 aa, synthetic construct
GYYRYEGAMDY

SEQ ID NO: 410 - protein, 11 aa, synthetic construct
WGQGTSVTVSS

SEQ ID NO: 411 - protein, 9 aa, synthetic construct
WGQGTSVTV

SEQ ID NO: 412 - protein, 112 aa, synthetic construct
NIVMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSWTFGGGTKLEIK

SEQ ID NO: 413 - protein, 23 aa, synthetic construct
NIVMTQSPSSLAVSAGEKVTMSC

SEQ ID NO: 414 - protein, 15 aa, synthetic construct
WYQQKPGQSPKLLIY

SEQ ID NO: 415 - protein, 7 aa, synthetic construct
WASTRES

SEQ ID NO: 416 - protein, 32 aa, synthetic construct
GVPDRFTGSGSGTDFTLTISSVQAEDLAVYYC

SEQ ID NO: 417 - protein, 8 aa, synthetic construct
HQYLSSWT

SEQ ID NO: 418 - protein, 12 aa, synthetic construct
FGGGTKLEIKTS

SEQ ID NO: 419 - protein, 10 aa, synthetic construct
FGGGTKLEIK

SEQ ID NO: 420 - protein, 15 aa, synthetic construct
GGGGSGGGGSGGGGS

SEQ ID NO: 421 - protein, 17 aa, synthetic construct
KSSQSVLYSSNQKNYLA

SEQ ID NO: 422 - protein, 5 aa, synthetic construct
GGGGS

(19) United States
(12) Patent Application Publication
(10) Pub. No.: US 2025/0235535 A1
(43) Pub. Date: Jul. 24, 2025

(54) COMPOSITIONS AND METHODS FOR THE TREATMENT OF VEN/AZA RESISTANT ACUTE MYELOID LEUKEMIA

(71) Applicant: The Regents of the University of Colorado, A Body Corporate, Denver, CO (US)

(72) Inventors: Mark Eric Kohler, Denver, CO (US); Haley Simpson, Denver, CO (US); Catherine Danis, Denver, CO (US); Lillie Leach, Denver, CO (US); Amanda Novak, Denver, CO (US)

(21) Appl. No.: 19/171,465

(22) Filed: Apr. 7, 2025

Related U.S. Application Data
(63) Continuation of application No. PCT/US2024/058938, filed on Dec. 6, 2024.
(60) Provisional application No. 63/610,215, filed on Dec. 14, 2023.

(51) Int. Cl. A61K 40/11; A61K 40/31; A61K 40/42; A61P 35/02; C07K 16/28; C12N 5/0783

(52) U.S. Cl. CPC A61K 40/11; A61K 40/31; A61K 40/421; A61P 35/02; C07K 16/283; C12N 5/0636; C07K 2317/622; C07K 2319/02; C07K 2319/03; C12N 2510/00

(57) ABSTRACT

The disclosure describes T cells that express chimeric antigen receptors (CARs), as well as pharmaceutical compositions comprising T cells and methods of making and using such T cells. Particularly, this disclosure describes T cells expressing a CAR that binds to CD64, and methods of use in treating acute myeloid leukemia.

### CROSS-REFERENCE TO RELATED APPLICATIONS

Pursuant to 35 U.S.C. § 119(e) this application is a continuation of International Application PCT/US2024/058938, filed on Dec. 6, 2024, which claims the benefit of U.S. Patent Application Ser. No. 63/610,215, filed on Dec. 14, 2023. The disclosure of the prior applications are considered part of (and are incorporated by reference in) the disclosure of this application.

### SEQUENCE LISTING

This application contains a Sequence Listing that has been submitted electronically as an XML file named “58666-0003001_SL_ST26.XML.” The XML file, created on Apr. 1, 2025, is 374,316 bytes in size. The material in the XML file is hereby incorporated by reference in its entirety.

### BACKGROUND OF THE INVENTION

Acute myeloid leukemia (AML) is a blood cancer in which the bone marrow of a subject makes abnormal myeloblasts, red blood cells, or platelets. AML is one of the most common forms of acute leukemia in adults. The build-up of AML cells in bone marrow and blood can rapidly lead to infection, anemia, excessive bleeding, and death. The BCL-2 inhibitor venetoclax has recently emerged as an important component of therapy for acute myeloid leukemia (AML). The current FDA-approved standard of care for the majority of patients who are too elderly or unfit for aggressive chemotherapy is treatment with venetoclax in combination with a hypomethylating agent, such as azacitidine (“ven/aza treatment”) or decitabine. It is estimated that approximately 70% of these patients will achieve complete remission (CR) of their disease upon ven/aza treatment. However, it is estimated that approximately 30% of patients do not respond to treatment with ven/aza and are unable to achieve CR. Furthermore, of those patients that initially achieve a CR, the majority will relapse in the following two years. Thus, there is a need in the art for alternative methods of treating subjects that will not respond to or relapse after treatment with ven/aza.

### SUMMARY OF INVENTION

The present disclosure provides polypeptides comprising: a) a first domain comprising an amino acid sequence of one of SEQ ID NO: 1, SEQ ID NO: 2 and SEQ ID NO: 3; b) a second domain comprising an amino acid sequence of SEQ ID NO: 4; c) a third domain comprising an amino acid sequence of SEQ ID NO: 5; d) a fourth domain comprising an amino acid sequence of SEQ ID NO: 6; and e) a fifth domain comprising an amino acid sequence of SEQ ID NO: 7.

In some aspects, the first domain further comprises an amino acid sequence of SEQ ID NO: 23.

In some aspects, the polypeptides comprise an amino acid sequence of one of SEQ ID NO: 11, SEQ ID NO: 12 and SEQ ID NO: 13.

The present disclosure provides polypeptide comprising: a) a first domain comprising an amino acid sequence of one of SEQ ID NO: 1, SEQ ID NO: 2 and SEQ ID NO: 3; b) a second domain comprising an amino acid sequence of SEQ ID NO: 8; c) a third domain comprising an amino acid sequence of SEQ ID NO: 9; d) a fourth domain comprising an amino acid sequence of SEQ ID NO: 10; and e) a fifth domain comprising an amino acid sequence of SEQ ID NO: 7.

In some aspects, the first domain further comprises an amino acid sequence of SEQ ID NO: 23.

In some aspects, polypeptides comprise an amino acid sequence of one of SEQ ID NO: 14, SEQ ID NO: 15 and SEQ ID NO: 16.

The present disclosure provides a nucleic acid molecules comprising nucleic acid sequences encoding a polypeptide of the present disclosure. In some aspects, the nucleic acid molecules comprise a nucleic acid sequence of one of SEQ ID NO: 17-22.

The present disclosure provides vectors comprising the nucleic acid molecules of the present disclosure, preferably wherein the vector is a viral vector, preferably wherein the viral vector is a lentiviral vector.

The present disclosure provides cells comprising the polypeptides of the present disclosure, the nucleic acid molecules of the present disclosure, and/or the vectors of the present disclosure.

In some aspects, the cells are immune cell, preferably wherein the immune cells are T-cells or Natural Killer (NK) cells.

The present disclosure provides populations of the cells of the present disclosure.

The present disclosure provides methods of treating Acute Myeloid Leukemia (AML) in a subject, the method comprising administering the populations of cells of the present disclosure.

In some aspects, the subjects having AML have a population of AML cells that are CD64+.

In some aspects, the subjects having AML have a population of monocytic leukemia stem cells (mLSCs).

In some aspects, the subjects have been previously administered at least one AML-targeting therapy, preferably wherein the AML-targeting therapy comprises the administration of a combination of venetoclax and azacitidine.

In some aspects, the subject has relapsed after treatment with the at least one AML-targeting therapy and/or the subject is resistant to treatment with the at least one AML-targeting therapy.

Any of the above aspects and embodiments, or aspects and embodiments described herein can be combined with any other aspect and embodiment.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers, or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

FIG. 1A-C is a series of graphs showing that CD64 CAR with a CD28 costimulatory domain and the m22 single chain fragment variable (scFv) (CD64-28z) T cells have antigen-dependent effector activity against AML cell lines. A) CD64 expression on AML cell lines K562 (CD64-negative), MOLM14 (CD64-low), and THP1 (CD64-high) used in downstream experiments (top). AML cell lines were co-incubated with either T cells from a healthy donor that were activated but not transduced (Mock, negative control) or with T cells transduced with a CD64 CAR containing a CD28 costimulatory domain (CD64 CAR). AML cells and T cells were co-incubated at a 1:1 ratio for 24 hours in triplicate and evaluated by flow cytometry. B) Quantification of the percentage of AML cells of remaining viable cells after the 24-hour coincubation using flow cytometry. The results demonstrate no reduction of the CD64-negative K652 cells by CD64 CAR T cells, but significant reduction of the CD64-low MOLM14 and CD64-high THP1 AML cells by CD64-28z CAR T cells (labeled as CD64 CAR). C) CD64-negative K562 cells and CD64-high THP1 cells were co-incubated with either CD19 CAR T cells (CD19-28z, irrelevant CAR used as a negative control) or with CD64 CAR T cells (CD64-28z) at a 1:1 ratio for 24 hours to measure antigen-dependent cytokine secretion from CD64-28z CAR T cells. CAR T cells were cultured alone as a negative control. All co-incubations/cultures were done in triplicate. After 24 hours, supernatant was collected from all wells and evaluated for cytokines by ELISA, demonstrating significant production of Interferon-gamma (IFNG) and Interleukin-2 (IL-2) by CD64-28z CAR T cells only in the presence of CD64+ AML cells. Statistical comparisons between CD64-28z and negative controls were performed using Mann-Whitney analysis.

FIG. 2A-B is a series of graphs showing that CD64-28z CAR T cells (containing the m22 scFv) have in vivo efficacy against CD64+ AML cells lines in xenograft models. A) NSG mice were inoculated with 10⁶ luciferase-expressing THP1 cells via tail vein injection on Day −4. Bioluminescent imagining (BLI) confirmed engraftment of the THP1 cells on Day −1. On Day 0, Mice were either treated with 5×10⁶ CD64-28z CAR T cells or were given no treatment. Leukemia burden was monitored by BLI once to twice weekly and mice were monitored for survival based on the development of predefined endpoints of terminal leukemia. CD64-28z CAR T cells significantly reduced leukemia progression and prolonged survival relative to no treatment. B) NSG mice were inoculated with 10⁶ luciferase-expressing MOLM14 cells via tail vein injection on Day −4. Bioluminescent imagining (BLI) confirmed engraftment of the MOLM14 cells on Day −1. On Day 0, Mice were either treated with 5×10⁶ CD64-28z CAR T cells or were given no treatment. Leukemia burden was monitored by BLI twice weekly, and mice were monitored for survival based on the development of predefined endpoints of terminal leukemia. CD64-28z CAR T cells significantly reduced leukemia progression and prolonged survival relative to no treatment. Statistical analysis of survival in all experiments per Log-Rank analysis with ** representing a p-value of less than 0.01.

FIG. 3A-C is a series of graphs showing that CD64-28z CAR T cells (containing the m22 scFv) have cytotoxicity against patient-derived monocytic AML in vitro. A) CD64+ Monocytic AML cells with monocytic leukemia stem cells (mLSCs) were obtained from the University of Colorado Biorepository. B-C) Mock T cells or CD64-28z CAR T cells derived from the same healthy donor were co-incubated in vitro with patient AML cells in triplicate for 24 or 48 hours at different effector (T cell) to target (AML cells) ratios (E:T ratios). B) Co-incubation with CD64-28z CAR T cells resulted in a significant, dose-dependent depletion of live AML cells at all E:T ratios, as measured by flow cytometry. C) Flow cytometric evaluation of remaining AML cells at 24 and 48 hours demonstrates CD64-28z CAR T cells specifically deplete CD64+ AML cells significantly more than mock T cells at all E:T ratios. Depletion of CD64+ AML cells by CD64-28z CAR T cells occurs in a dose-dependent manner and increases from 24 hours to 48 hours at E:T ratios of 1:4 and 1:16, whereas all CD64+ AML cells are depleted within the first 24 hours at the 1:1 E:T ratio. Statistical comparisons using two-way ANOVA with Tukey's multiple comparison test. ns=not significant, ***=p-value less than 0.001, ****=p-value less than 0.0001.

FIG. 4A-C is a series of graphs showing that CD64-28z CAR T cells (containing the m22 scFv) have cytotoxicity against patient-derived AML with mixed primitive and monocytic populations in vitro. A) Patient derived AML with a mixed population of CD64− (primitive) and CD64+(monocytic) leukemia stem cells (LSCs) were obtained from the University of Colorado Biorepository. B-C) Mock T cells or CD64-28z CAR T cells derived from the same healthy donor were co-incubated with patient AML cells in vitro for 24 or 48 hours at different effector (T cell) to target (AML cells) ratios (E:T ratio) in triplicate. B) Co-incubation with CD64-28z CAR T cells resulted in a significant, dose-dependent depletion of live AML cells at all E:T ratios, as measured by flow cytometry. C) Flow cytometric evaluation of remaining AML cells at 24 and 48 hours demonstrates CD64-28z CAR T cells specifically deplete CD64+ AML cells significantly more than mock T cells at all E:T ratios. Depletion of CD64+ AML cells by CD64-28z CAR T cells occurs in a dose-dependent manner and increased from 24 hours to 48 hours for E:T ratios of 1:4 and 1:16, whereas all CD64+ AML cells were depleted within the first 24 hours at the 1:1 E:T ratio. Statistical comparisons using two-way ANOVA with Tukey's multiple comparison test. ns=not significant, *=p-value less than 0.05, ****=p-value less than 0.0001.

FIG. 5A-D is a series of graphs showing that CD64-28z CAR T cells (containing the m22 scFv) deplete monocytic leukemic stem cells (mLSCs) from patient-derived AML samples. Patient-derived AML samples were obtained from the University of Colorado Biorepository. Predominantly monocytic AML samples (FIGS. 5A-5B) and mixed primitive/monocytic AML patient samples (FIGS. 5C-5D) were co-incubated with CD64-28z CAR T cells or with matched Mock T cells at an Effector:Target ratio of 1:2 for 24 hours, in vitro. T cells were antibody-depleted and remaining AML cells were injected into NSG-S mice (10⁶ AML cells/mouse; n=7-10 mice/group). AML was allowed to engraft over the next 10 weeks, at which point the mice were taken down and the bone marrow was evaluated by flow cytometry for engraftment of human AML cells and for CD64+ human AML cells. We observed a significant decrease in the engraftment of monocytic AML after pre-incubation with CD64-28z CAR T cells relative to mock T cells (FIG. 5A) with a significant reduction of CD64+ AML cells within the engrafted population (FIG. 5B). We similarly observed a decreased engraftment of mixed primitive/monocytic AML cells after pre-incubation with CD64-28z CAR T cells relative to mock T cells (FIG. 5C) with a significant reduction of CD64+ AML cells as well (FIG. 5D). Collectively, the decreased engraftment of the bulk population and the CD64+ AML cells after ex vivo CAR incubation demonstrates the ability of CD64 CAR T cell to target a mLSC population which carries the engraftment potential of the leukemic population. Statistical comparisons using Mann-Whitney Analysis as indicated.

FIG. 6A-C is a series of graphs showing that a CD64 CAR containing the m22 scFv and a 4-1BB costimulatory domain (CD64-BBz) and a CD64 CAR containing the m22 scFv and a CD28 costimulatory domain (CD64-28z) CAR T cells have in vivo efficacy against CD64+AML cells lines in xenograft models. A-B) NSG mice were inoculated with 10⁶ luciferase-expressing MOLM14 cells via tail vein injection on Day −4. Bioluminescent imagining (BLI) confirmed engraftment of the MOLM14 cells on Day −1. On Day 0, Mice were either treated with 3×10⁶ CD64-28z CAR T cells, 3×10⁶ CD64-BBz CAR T cells or an equivalent number of Mock T cells. Mice were treated with Mock or CAR T cells from three independent T cell donors (5 mice per treatment per T cell donor). Leukemia burden was monitored by BLI once to twice weekly (FIG. 6A) and mice were monitored for survival based on the development of predefined endpoints of terminal leukemia (FIG. 6B). Both CD64-28z and CD64-BBz CAR T cells significantly reduced leukemia progression and prolonged survival relative to Mock T cells, with CD64-BBz CAR T cells demonstrating a survival advantage over treatment with CD64-28z CAR T cells. Statistical analysis of survival of each group, combined between donors was performed using Log-Rank analysis whereas *=p-value less than 0.05, and ****=p-value less than 0.0001. C) Quantification of bioluminescent signal as total flux per mouse (photons/second) to quantify leukemia burden in mice over time. Mice treated with CD64-BBz and CD64-28z CAR-T cells demonstrated decreased leukemia burden over time compared to mock treated mice. Statistical analysis by two-way ANOVA where *=p-value less than 0.05, and ****=p-value less than 0.0001. The difference between CD64-28z and CD64-BBz CAR T cell treatment showed a p-value of 0.06 by two-way ANOVA.

FIG. 7A-B is a series of graphs showing CD64 expression in various leukemia models. A) A series of histograms showing expression of CD64 measured by flow cytometry on K562, MOLM14, and THP-1 AML cell lines. B) A series of histograms and contour plots showing characterization of CD64 expression in primary AML samples.

FIG. 8A-D is a series of diagrams and graphs demonstrating different versions of the CD64 CAR can be expressed in healthy donor T-cells. A) Schematic of exemplary CD64-28-m22, CD64-BB-m22, and CD64-BB-611 CAR constructs. The CD64-28-m22 consists of the m22 scFv, an extracellular and transmembrane (EC/TM) domain derived from CD28, a CD28 costimulatory domain, and the CD3-zeta (CD3z) signaling domain. The CD64-BB-m22 consists of the m22 scFv, an extracellular and transmembrane (EC/TM) domain derived from CD8, a 4-1BB costimulatory domain, and the CD3-zeta (CD3z) signaling domain. The CD64-BB-611 consists of the 611 scFv, an extracellular and transmembrane (EC/TM) domain derived from CD8, a 4-1BB costimulatory domain, and the CD3-zeta (CD3z) signaling domain. B) Histograms showing CD64-28-m22, CD64-BB-m22, and CD64-BB-611 CAR construct transduction efficiency and expression in healthy donor human T cells. C) Graph of CD4 to CD8 T cell ratios in CD64-BB-611, CD64-BB-m22, and CD64-28-m22 CAR T cell populations. D) Bar graph comparing T cell phenotypes in CD64-BB-611, CD64-BB-m22, and CD64-28-m22 CAR T cell populations. Statistical analysis by two-way ANOVA, where **=p-value of less than 0.01, ***=p-value of less than 0.001 and ****=p-value of less than 0.0001.

FIG. 9A-E is a series of graphs demonstrating that CD64 CAR T cells are cytotoxic against CD64+ AML cells in vitro. A) Scatter plots of flow cytometry results assessing residual K562, MOLM14, and THP-1 AML cell lines cells after co-culture with CD64-BB-611, CD64-BB-m22, and CD64-28-m22 CAR T cells. B) Quantification of killing of AML cell line cells as shown in FIG. 9A from three replicates at two different E:T ratios showing antigen-dependent cytotoxicity of all CD64 CAR constructs. Statistical analysis by two-way ANOVA, where **=p-value of less than 0.01, ***=p-value of less than 0.001 and ****=p-value of less than 0.0001. C) Luciferase-based killing assay assessing CD64-BB-611, CD64-BB-m22, and CD64-28-m22 CAR T cell killing of K562, MOLM14, and THP-1 AML cell line cells. Results show each CD64 CAR construct produces antigen-dependent cytotoxicity. D) Scatter plots showing CD64-BB-611, CD64-BB-m22, and CD64-28-m22 CAR T cell reduction of CD64⁺ AML cells from patient-derived monocytic and mixed monocytic/primitive (“Mixed”). E) Quantification of CD64-BB-611, CD64-BB-m22, and CD64-28-m22 CAR T cell killing of monocytic and mixed monocytic/primitive AML patient samples after 24-hour co-culture at various E:T ratios. Cytotoxicity calculated based on normalization to AML cells co-cultured with equivalent number of Mock T cells at the same E:T ratio and analyzed by flow cytometry at the end of the 24-hour co-culture.

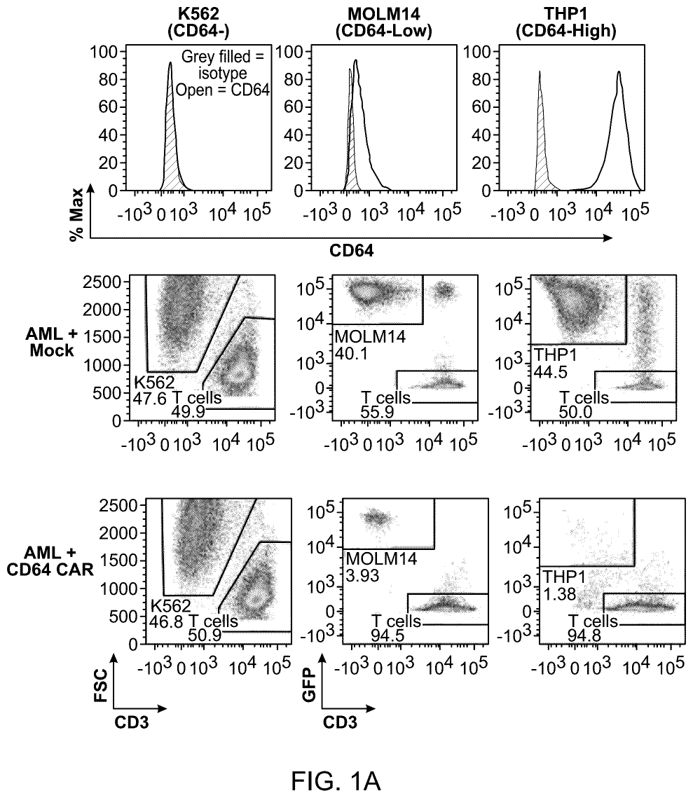

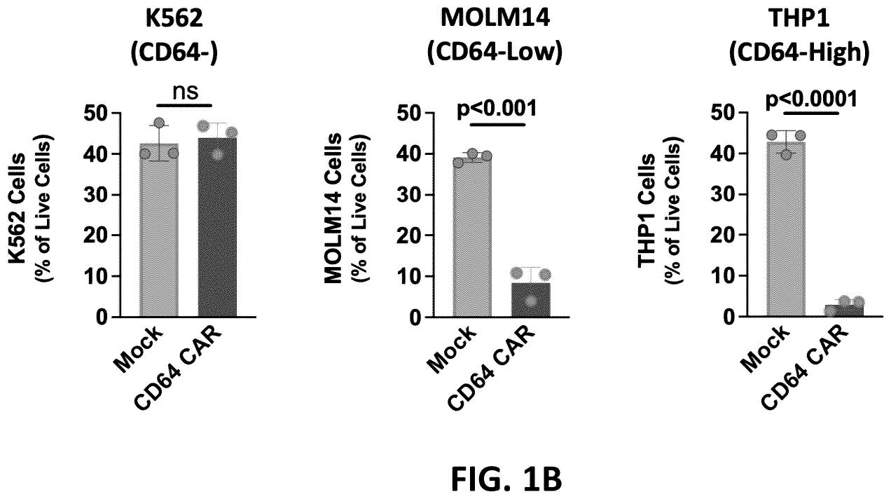

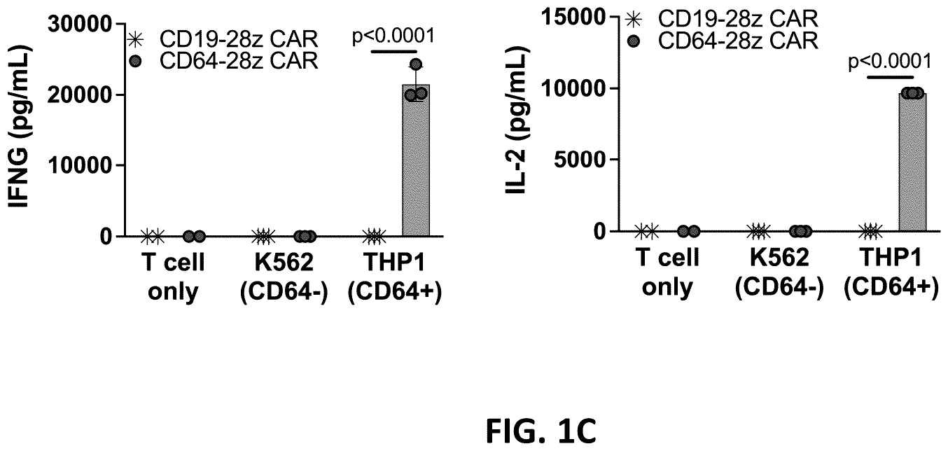

FIG. 1A-F is a series of graphs showing that T cell effector cytokines are released when CD64 CAR T cells engage CD64⁺ AML cells. A-C) Bar graphs showing antigen-dependent secretion of Interleukin-2 (IL-2) (FIG. 10A), Interferon-gamma (IFNg) (FIG. 10B), and tumor necrosis factor alpha (TNFa) (FIG. 10C) by CD64-BB-m22 and CD64-28-m22, and CD64-BB611 CAR T cells after 24-hour co-culture with the AML cell lines K562, MOLM14, and THP1. D-F) Bar graphs showing antigen-dependent secretion of IL-2 (FIG. 10D), IFNg (FIG. 10E), and TNFa (FIG. 10F) by CD64-BB-m22 and CD64-28-m22, and CD64-BB611 CAR T cells after 24-hour co-culture with patient-derived monocytic or mixed primitive/monocytic AML samples. Statistical analysis throughout FIG. 10 done by two-way ANOVA, where *=p-value of less than 0.05, **=p-value of less than 0.01, ***=p-value of less than 0.001 and ****=p-value of less than 0.0001.

FIG. 11A-E is a series of graphs showing the manufacturing and efficacy of patient-derived CD64 CAR T cells relative to healthy donor CD64 CAR T cells. A) Line graph showing proliferation of T cells during manufacturing of CD64-BB-m22 CAR T cells. This data demonstrates that patient-derived T cells proliferate with comparable kinetics as healthy donor-derived T cells during CD64-BB-m22 CAR T cell manufacturing. B) Histograms showing CD64-BB-m22 CAR construct transduction efficiency of T cells from two AML patient donors (pt-AML03 and pt-AML06) as compared to T cells from two health donors (Healthy Donor 42 and 20). C) Bar graph comparing proliferation of patient-derived CD64-BB-m22 CAR T cells and healthy donor-derived CD64-BB-m22 CAR T cells after 24-hour co-culture with MOLM14 AML cells. D) Luciferase killing assay assessing overnight killing of MOLM14 AML cells by two patient-derived CD64-BB-m22 CAR T cell products demonstrating patient-derived CAR T cells are cytotoxic. E) Scatter plots and histograms showing elimination of CD64⁺ AML cells from monocytic and mixed patient-derived AML samples by autologous patient-derived CD64-BB-m22 CAR T cells.

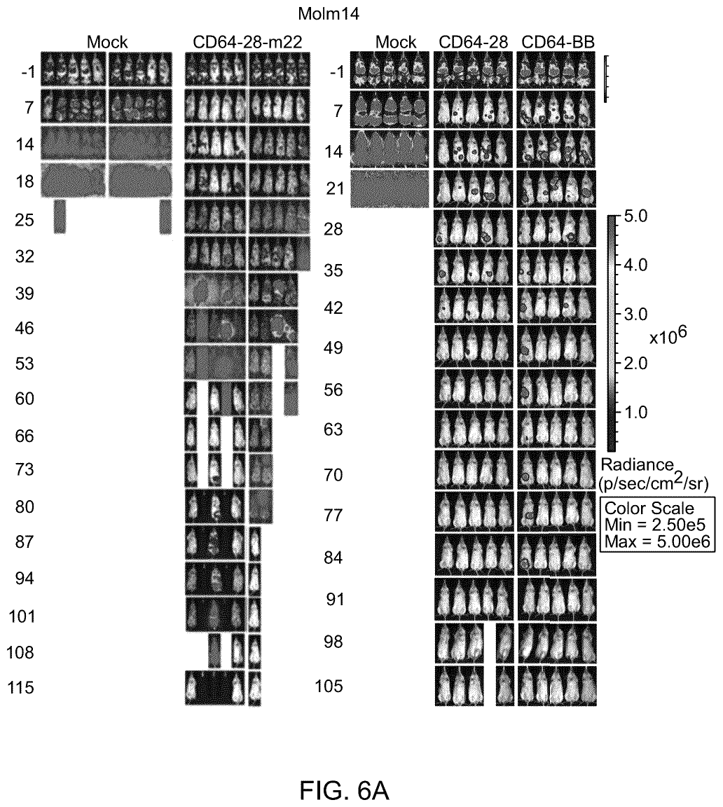

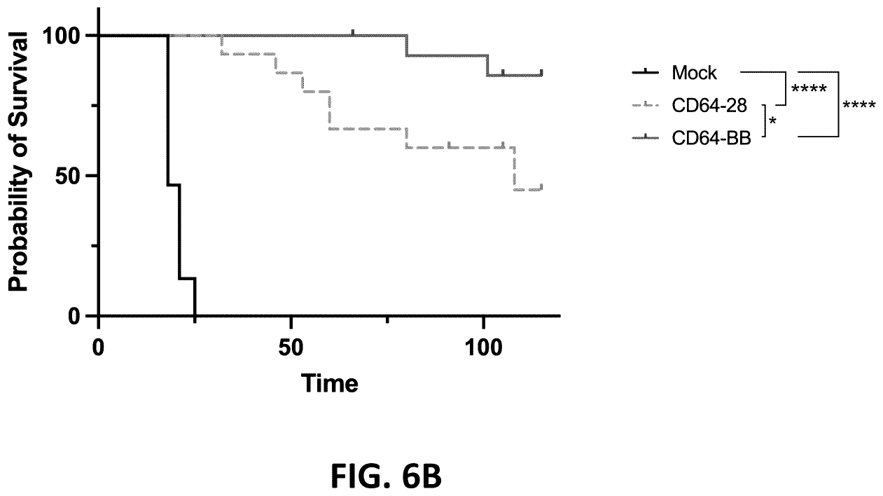

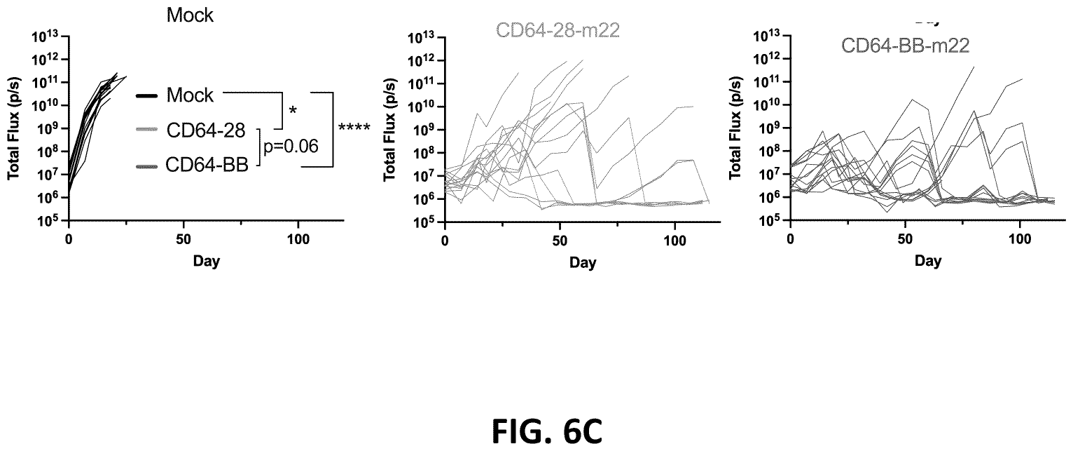

FIG. 12A-F is a series of bioluminescent images and graphs showing CD64 CAR T cells eliminate CD64⁺ AML cell lines in xenograft models. A) Bioluminescent images of THP-1 leukemia burden in NSG mice engrafted with 10⁶ THP1 cells on Day −4 and treated with 5×10⁶ CD64-BB-611, CD64-BB-m22, or CD64-28-m22 (labeled as CD64-28) CAR T cells, or an equivalent dose of Mock T cells from the same T cell donor. This experiment was performed in triplicate with three independent healthy T cell donors. B) Survival analysis of mice treated with CD64-BB-611, CD64-BB-m22, or CD64-28-m22 CAR T cells, or mock-treated control. Both CD64-BB-611 and CD64-BB-m22 CAR T cells significantly reduced leukemia progression and prolonged survival relative to Mock T cell and CD64-28z-m22 CAR T cell treated mice. Statistical analysis of survival of each group, combined between donors was performed using Log-Rank analysis, where **=p-value of less than 0.01, ***=p-value of less than 0.001 and ****=p-value of less than 0.0001. C) Quantification of bioluminescent signal as total flux per mouse (photons/second) to quantify leukemia burden in mice over time. Mice treated with CD64-BB-m22 and CD64-BB-611 CAR-T cells demonstrated decreased leukemia burden compared to mock treated mice. Statistical analysis by two-way ANOVA, where *=p-value of less than 0.05, **=p-value of less than 0.01, ***=p-value of less than 0.001 and ****=p-value of less than 0.0001. D) Single T-cell donor replicate of data as shown in FIG. 6 with inclusion of treatment with CD64-BB-611 in NSG mice engrafted with MOLM14 leukemia. Bioluminescent imaging of MOLM14 leukemia burden in NSG mouse xenografts models treated with 3×10⁶ CD64-BB-611, CD64-BB-m22, and CD64-28-m22 CAR T cells as compared to mock-treated control. E) Survival analysis of mice treated with CD64-BB-611, CD64-BB-m22, or CD64-28-m22 CAR T cells, or mock-treated control, showing prolonged survival after treatment with any version of the CD64 CAR compared to Mock T cells. Statistical analysis of survival of each group, combined between donors was performed using Log-Rank analysis, where **=p-value of less than 0.01, ***=p-value of less than 0.001 and ****=p-value of less than 0.0001. F) Quantification of bioluminescent signal as total flux per mouse (photons/second) to quantify leukemia burden in mice over time. Mice treated with CD64-28-m22, CD64-BB-m22, and CD64-BB-611 CAR-T cells demonstrated decreased leukemia burden compared to mock treated mice. Statistical analysis by two-way ANOVA, where *=p-value of less than 0.05, **=p-value of less than 0.01, ***=p-value of less than 0.001 and ****=p-value of less than 0.0001.

FIG. 13A-F is a series of graphs showing that CD64 CAR T cells persist after clearance of CD64⁺ leukemia in xenograft models. A) Graphs showing percentage and absolute number of total, CD4⁺, and CD8⁺CD64-BB-m22 and CD64-28-m22 CAR T cells persisting in the bone marrow at 35 days post-infusion and after MOLM14 leukemia clearance. Statistical analysis by Mann-Whitney, where *=p-value of less than 0.05, **=p-value of less than 0.01, ***=p-value of less than 0.001 and ****=p-value of less than 0.0001. B) Bar graph comparing T cell phenotypes of persisting CD64-BB-m22 and CD64-28-m22 CAR T cells at 35 days post-infusion and MOLM14 leukemia clearance. C) Bar graph showing percentages of persisting CD64-BB-m22 and CD64-28-m22 CAR T cells expressing PD1 and TIM3 exhaustion markers 35 days post-infusion and after MOLM14 leukemia clearance. D) Graphs showing percentage and absolute number of total, CD4⁺, and CD8⁺CD64-BB-611, CD64-BB-m22, and CD64-28-m22 CAR T cells persisting in the bone marrow 105 days after infusion and after MOLM14 leukemia clearance. Statistical analysis by one-way ANOVA, where *=p-value of less than 0.05, **=p-value of less than 0.01, ***=p-value of less than 0.001 and ****=p-value of less than 0.0001. E) Bar graph comparing T-cell phenotypes of persisting CD64-BB-611, CD64-BB-m22, and CD64-28-m22 CAR T cells 105 days after infusion and after MOLM14 leukemia clearance. Statistical analysis by two-way ANOVA, where *=p-value of less than 0.05. F) Bar graph showing percentages of persisting CD64-BB-611, CD64-BB-m22, and CD64-28-m22 CAR T cells expressing PD1 and TIM3 exhaustion markers 105 days after infusion and after MOLM14 leukemia clearance.

FIG. 14A-E is a series of graphs showing that CD64 CAR T cells exhibit toxicity against monocytes. A) Flow cytometry scatter plots showing the elimination of healthy donor-derived monocytes by CD64-BB-611, CD64-BB-m22, and CD64-28-m22 CAR T cells after overnight co-culture. B) Quantification of monocyte killing in two independent monocyte/T cell donors, in which CD64-28-m22, CD64-BB-m22, CD64-BB-611 CAR T cells were co-cultured with autologous monocytes at various E:T ratios overnight and cytotoxicity of monocytes was calculated by flow cytometry in comparison to Mock T cell negative control. C-E) Bar graphs showing antigen-dependent secretion of IL-2 (FIG. 14C), IFNg, (FIG. 14D), and TNFa (FIG. 14E) by CD64-BB-m22, CD64-28-m22, and CD64-BB-611 CAR T cells after overnight co-culture with monocytes. Three independent healthy T cell donors were used for CAR T cell production (20, 28, and 42). Statistical analysis by two-way ANOVA, where *=p-value of less than 0.05, **=p-value of less than 0.01, ***=p-value of less than 0.001 and ****=p-value of less than 0.0001.

FIG. 15A-F is a series of graphs that show CD64 CAR T cells in combination with venetoclax and azacitidine additively eliminate CD64⁺ primary AML ex vivo. A-B) Graphs showing remaining viable AML population (FIG. 15A) and CD33, CD34, and CD64 expression (FIG. 15B) in mixed monocytic/primitive AML cells treated with venetoclax and azacitadine (ven/aza), CD64-BB-m22 CAR T cells, both ven/aza and CD64-BB-m22 CAR T cells, or neither ven/aza or CD64-BB-m22 CAR T cells (DMSO used as negative control for ven/aza and mock T cells used as negative control for CAR T cells). C) Graph showing the quantification of residual CD64+ and CD64− AML cells from a primary mixed monocytic/primitive AML patient sample following treatment with ven/aza, CD64-BB-m22 CAR T cells, both ven/aza and CD64-BB-m22 CAR T cells, or neither ven/aza or CD64-BB-m22 CAR T cells (DMSO/Mock control). D-E) Graphs showing the remaining viable AML population (FIG. 15D) and CD33, CD34, and CD64 expression (FIG. 15E) on these residual AML cells after treatment of a monocytic AML patient sample with ven/aza, CD64-BB-m22 CAR T cells, both ven/aza and CD64-BB-m22 CAR T cells, or neither ven/aza or CD64-BB-m22 CAR T cells (DMSO/Mock control). F) Graph showing the quantification of CD64+ and CD64− AML cells from a primary monocytic AML patient sample after treatment with ven/aza, CD64-BB-m22 CAR T cells, both ven/aza and CD64-BB-m22 CAR T cells, or DMSO and Mock T cell negative controls. Statistical analysis of FIGS. 15C and 15F done by two-way ANOVA, where *=p-value of less than 0.05, **=p-value of less than 0.01, ***=p-value of less than 0.001 and ****=p-value of less than 0.0001.

### DETAILED DESCRIPTION OF THE INVENTION

Acute myeloid leukemia (AML) is a blood cancer that is one of the most commonly diagnosed types of leukemia in adults. There were an estimated 11,000 deaths from AML in the United States in 2020, along with 20,000 newly diagnosed cases. The average age of a person diagnosed with acute myeloid leukemia is about 68, with most cases occurring after the age of 45. However, acute myeloid leukemia is also diagnosed in younger patients, and is the second most common leukemia in children. Prognosis for patients diagnosed with acute myeloid leukemia is generally poor, with a long-term survival of only 40-50% in younger adult patients and a median overall survival of less than one year for older patients. New therapies aimed at supplementing the standard remission induction regimen of cytarabine with intermittent dosing of an anthracycline have not yielded additional clinical benefits. Thus, there exists a need for more specialized and personalized treatment methods, particularly in older patients who are unfit for induction therapy.

Recent research has demonstrated that acute myeloid leukemia exhibits a high level of biological heterogeneity, potentially explaining the difficulty in finding effective therapeutic strategies for the treatment of AML. Furthermore, it has been recently recognized that leukemia stem cells (LSCs), which are capable of giving rise to identical as well as differentiated daughter cells, perpetuate and maintain acute myeloid leukemia and promote disease relapses.

As an alternative to high intensity induction therapy, the current FDA-approved standard of care for elderly patients or patients who are otherwise unfit for aggressive chemotherapy is treatment with a combination of the BCL-2 inhibitor venetoclax and a hypomethylating agent (HMA), such as azacitidine or decitabine. Specifically, treatment with a combination of venetoclax and azacitidine (hereafter referred to as “ven/aza treatment” or “treatment with ven/aza”) is estimated to induce a complete remission (CR) of AML in approximately 70% of treated patients. However, this means that approximately 30% of patients do not end up responding to ven/aza treatment and therefore do not achieve complete remission. Furthermore, on average, this remission lasts approximately 18 months with patients ultimately relapsing and dying from leukemia or complications of leukemia-directed therapy. Accordingly, there is a need in the art for new therapies for the treatment of AML, specifically subjects that do not respond to ven/aza treatment or that relapse after treatment with ven/aza.

The present disclosure is based on, inter alia, the discovery that the cell surface protein Cluster of Differentiation 64 (“CD64”) can be used to target cellular therapies for the eradication of specific subpopulations of AML cells. Accordingly, the present disclosure provides anti-CD64 cellular immunotherapies for the treatment of AML in a subject.

The present invention generally provides cells, including immune cells (e.g., T cells, B cells, Natural Killer (NK) cells, monocytes, macrophages, or artificially generated cells with immune effector function) derived from a patient, a healthy donor, a differentiated stem cell (including but not limited to induced pluripotent stem cells (iPSC), embryonic stem cells, hematopoietic and/or other tissue specific stem cells), or a non-human source, which are genetically modified to express an antigen recognizing receptor (e.g., chimeric antigen receptor (CAR)) that binds to CD64 and methods of use thereof for the treatment of acute myeloid leukemia in a subject. Immune cell (e.g., T cell) activation is mediated by engagement of CAR molecules to its cognate antigen (i.e., CD64) with signal amplification leading to enhanced persistence, antigen-sensitivity, and efficacy occurring when the CAR is engaged to its respective cognate.

CARs, which are at times referred to as artificial T cell receptors, chimeric T cell receptors (cTCR), T-bodies or chimeric immunoreceptors, are engineered receptors known in the art. They are used primarily to transform immune effector cells, in particular T cells, to provide those cells with a desired antigen specificity and effector response. Adoptive cell therapies using CAR T cells are particularly under investigation in the field of cancer therapy. In these therapies, T cells are removed from a patient, donor or are derived from a stem cell source and engineered to express CARs specific to the antigens found in a particular form of cancer. The CAR T cells, which can then recognize and kill the cancer cells, are reintroduced into the patient whereupon the CAR T cells undergo proliferative expansion, elimination of target antigen-positive cells and, in a minority of patients, transition to a long-lasting, persistent population with retained anti-tumor effector activity.

First generation CARs provide a TCR-like signal from an Immunoreceptor Tyrosine-based Activation Motif (ITAM) containing intracellular signaling domain, most commonly derived from the CD3 zeta (CD3z) molecule, and thereby elicit tumoricidal functions. However, the engagement of CD3z-chain fusion receptors may not suffice to elicit substantial IL-2 secretion and/or T cell proliferation in the absence of a concomitant co-stimulatory signal. In physiological T cell responses, optimal lymphocyte activation requires the engagement of one or more co-stimulatory receptors such as CD28 or 4-1BB. In the setting of suboptimal activation elicited by first generation CARs, T cell activity in vivo is often transient and incapable of controlling the malignancy.

Second (2nd) generation CARs have been constructed to transduce a functional antigen-dependent co-stimulatory signal in human primary T cells in addition to antigen-dependent TCR-like signal, permitting T cell proliferation in addition to tumoricidal activity. Second generation CARs most commonly provide co-stimulation using co-stimulatory domains (synonymously, co-stimulatory signaling regions) derived from CD28 or 4-1BB. The combined delivery of co-stimulation plus a CD3 zeta signal renders 2nd generation CARs superior in terms of function as compared to their first generation counterparts (CD3z signal alone). An example of a 2nd generation CAR is found in U.S. Pat. No. 7,446,190, incorporated herein by reference.

Third (3rd) generation CARs have also been prepared. These combine multiple co-stimulatory domains (synonymously, co-stimulatory signaling regions) with a TCR-like signaling domain in cis, such as CD28+4-1BB+CD3z or CD28+OX40+CD3z, to further augment potency. In the 3rd generation CARs, the co-stimulatory domains are aligned in series in the CAR endodomain and are generally placed upstream of CD3z or its equivalent. In general, however, the results achieved with these third generation CARs have been disappointing, showing only a marginal improvement over 2nd generation configurations, with some 3rd generation CARs being inferior to 2nd generation configurations.

This present invention provides various CARs that engage with CD64. The present disclosure also provides immune cells (e.g., T cells) genetically engineered to express the CARs described herein. As demonstrated in the experimental examples herein, these immune cells comprising the CARs of the present disclosure demonstrate superior activity against acute myeloid leukemia (AML), including AML subtypes that are resistant to treatment with a combination of venetoclax and azacitidine. Thus, the present invention overcomes problems associated with treating the patients that do not end up responding to ven/aza treatment and therefore do not achieve complete remission.

### Chimeric Antigen Receptors (CARs) of the Present Disclosure

In some aspects, the present disclosure provides a polypeptide (i.e., a CAR) comprising: a) a first domain; b) a second domain; c) a third domain; d) a fourth domain; and e) a fifth domain. In some aspects, the polypeptide comprises an antigen recognition domain that specifically binds to CD64.

In some aspects of the preceding polypeptides, the first domain can comprise, consist essentially of, or consist of an antigen recognition domain.

In some aspects of the preceding polypeptides, the second domain can comprise, consist essentially of, or consist of a hinge domain.

In some aspects of the preceding polypeptides, the third domain can comprise, consist essentially of, or consist of a transmembrane domain, as described herein.

In some aspects of the preceding polypeptides, the fourth domain can comprise, consist essentially of, or consist of a costimulatory domain, as described herein.

In some aspects of the preceding polypeptides, the firth domain can comprise, consist essentially of, or consist of an activation domain, as described herein.

### Antigen Recognition Domains

Antigen recognition domain, (also referred to as an “Antigen recognition moiety”) refers to a molecule or portion of a molecule that specifically binds to an antigen. The antigen recognition domains of the present disclosure can specifically bind to CD64.

In some embodiments, the antigen recognition domain of the CAR described herein binds (e.g. specifically binds) to the antigen CD64 described in Table 1. The antigen specific CAR, when expressed on the cell surface, redirects the specificity of immune cells (e.g. T cells) to the respective antigen.

TABLE 1
Target of Antigen Recognition Domains

| | Protein Name | Protein Relevance | UniProt ID | NCBI Accession No. |
|---|---|---|---|---|
| CD64 | high affinity | CD64 is a type of | P12314 | NM_000566 |
| | immunoglobulin gamma Fc | integral membrane | | NM_001378804 |
| | receptor I; Fc fragment of | glycoprotein known as | | NM_001378805 |
| | IgG, high affinity Ia, | an Fc receptor that binds | | NM_001378806 |
| | receptor (CD64); Fc | monomeric IgG-type | | NM_001378807 |
| | fragment of IgG, high | antibodies with high | | NM_001378808 |
| | affinity Ia, receptor for | affinity. After binding | | NM_001378809 |
| | (CD64); Fc gamma receptor | IgG, CD64 interacts | | NM_001378810 |
| | Ia; Fc-gamma RI; Fc- | with an accessory chain | | NM_001378811 |
| | gamma receptor I A1; IgG | known as the common γ | | |
| | Fc receptor I; fc-gamma | chain (γ chain), which | | |
| | RIA; fcgammaRIa; Fc | possesses an ITAM | | |
| | fragment of IgG receptor Ia; | motif that is necessary | | |
| | CD64; FCRI; CD64A; | for triggering cellular | | |
| | IGFR1 | activation | | |

In some aspects, the antigen recognition domain of a CAR of the present disclosure is an antibody, antibody-like molecule, or fragment thereof and the antigen is a tumor antigen.

In some aspects. the antigen recognition domain of the CARs described herein may recognize an epitope comprising the shared space between one or more antigens. In some embodiments, the antigen recognition domain comprises complementary determining regions (CDRs) of a monoclonal antibody, variable regions of a monoclonal antibody, an scFv, a VH, a VHH, a single domain antibody (e.g., a camelid single domain antibody), an antibody mimetic and/or antigen binding fragments thereof. In some embodiments, the specificity of the antigen recognition domain is derived from a protein or peptide (e.g., a ligand in a receptor-ligand pair) that specifically binds to another protein or peptide (e.g., a receptor in a receptor-ligand pair). In some embodiments, the antigen recognition domain comprises an aptamer, a T cell receptor (TCR)-like antibody, or a single chain TCR (scTCR). Almost any moiety that binds a given target (e.g., tumor associated antigen (TAA)) with sufficient affinity can be used as an antigen recognition domain. The arrangement of the antigen recognition domain could be multimeric, such as a diabody or multimers. In some embodiments, the multimers can be formed by cross pairing of the variable portion of the light and heavy chains into a diabody.

In some embodiments, the antigen recognition domain of the CARs described herein comprises an antibody mimetic. The term “antibody mimetic” is intended to describe an organic compound that specifically binds a target sequence and has a structure distinct from a naturally-occurring antibody. Antibody mimetics may comprise a protein, a nucleic acid, or a small molecule. The target sequence to which an antibody mimetic of the disclosure specifically binds may be an antigen. Exemplary antibody mimetics include, but are not limited to, an affibody, an afflilin, an affimer, an affitin, an alphabody, an anticalin, an avimer (also known as avidity multimer), a DARPin (Designed Ankyrin Repeat Protein), a Fynomer, a Kunitz domain peptide, a monobody and a centyrin.

In some embodiments, the antigen recognition domains of the CARs provided herein comprise a single chain variable fragments (scFv) derived from monoclonal antibodies specific for CD64. Accordingly, the antigen recognition domain of a CAR provided herein can comprise any scFv known in the art to specifically bind CD64. In some embodiments, the antigen recognition domain of the CARs provided herein comprises a fragment of the VH and VL chains of a single-chain variable fragment (scFv) that specifically bind CD64.

In some embodiments, the antigen recognition domain of a CAR provided herein comprises an antibody or an antigen-binding fragment thereof. In some embodiments, the antigen recognition domain of a CAR provided herein comprises a single chain antibody fragment (scFv) comprising a light chain variable domain (VL) and heavy chain variable domain (VH) of a monoclonal anti-CD64 antibody. Optionally, the VH and VL may be joined by a flexible linker, such as a glycine-serine linker or a Whitlow linker. In some embodiments, the antigen binding moiety may comprise VH and VL that are directionally linked, for example, from N to C terminus, VH-linker-VL or VL-linker-VH.

In some embodiments, the antigen recognition domain of a CAR provided herein comprises an scFv whose affinity for CD64 has been optimized to induce cytotoxicity of tumor cells that produce high levels or normal levels of CD64. In some embodiments, the antigen recognition domain of a CAR provided herein comprises an scFv whose affinity for CD64 has been optimized to induce cytotoxicity of tumor cells that produce low levels of CD64.

In some embodiments, the antigen recognition domain of a CAR described herein comprises complementarity determining regions (CDRs) and/or a heavy chain variable domain (VH) and a light chain variable domain (VL) derived from an anti-CD64 antibody.

In some embodiments, the antigen recognition domain of a CAR described herein can comprise, consist essentially of, or consist of an amino acid sequence at least 65%, 70%, 75%, 80%, 85%, 90%, 95%, 96%, 97%, 98%, 99%, or 100% (or any percentage in between) identical to SEQ ID NO: 1, SEQ ID NO: 2, or SEQ ID NO: 3.

The antigen recognition domain of the CARs provided herein may include CDRs and/or VH and VL derived from an anti-CD64 antibody (or antigen binding fragment thereof). Anti-CD64 antibodies of the disclosure can comprise any one of the partial light chain sequences known in the art and/or any one of partial heavy chain sequences known in the art. In some embodiments, the antigen recognition domain of a CAR described herein comprises an scFv comprising a VH and a VL, wherein the VH comprises the amino acid sequence of a VH from an anti-CD64 antibody known in the art, and the VL comprises the amino acid sequence of the corresponding VL known in the art.

In some embodiments, the antigen recognition domain of a CAR described herein comprises an scFv comprising a VH and a VL, wherein the VH comprises a CDRH1, a CDRH2, and a CDRH3 each comprising the amino acid sequence of a CDRH1, a CDRH2, and a CDRH3 of an anti-CD64 antibody known in the art, and wherein and the VL comprises a CDRL1, a CDRL2, and a CDRL3 each comprising the amino acid sequence of a CDRL1, a CDRL2, and a CDRL3 of the same anti-CD64 antibody known in the art. Determination of CDR regions is well within the skill of the art. It is understood that in some embodiments, CDRs can be a combination of the Kabat and Chothia CDR (also termed “combined CRs” or “extended CDRs”).

In some embodiments, the CDRs are the Kabat CDRs. In other embodiments, the CDRs are the Chothia CDRs. In other embodiments, the CDRs are IMGT CDRs. In other words, in embodiments with more than one CDR, the CDRs may be any of Kabat, Chothia, IMGT combination CDRs, or combinations thereof.

In some embodiments, an antigen recognition domain of a CAR provided herein can include (i) a VH having a CDRH1 having the amino acid sequence set forth in SEQ ID NO:24 (or a derivative of SEQ ID NO:24 with one, two, three, or four amino acid modifications), a CDRH2 having the amino acid sequence set forth in SEQ ID NO:25 (or a derivative of SEQ ID NO:25 with one, two, three, or four amino acid modifications), and a CDRH3 having the amino acid sequence set forth in SEQ ID NO:26 (or a derivative of SEQ ID NO:26 with one, two, three, or four amino acid modifications), and/or (ii) a VL having a CDRL1 having the amino acid sequence set forth in SEQ ID NO:32 (or a derivative of SEQ ID NO:32 with one, two, three, or four amino acid modifications), a CDRL2 having the amino acid sequence set forth in SEQ ID NO:33 (or a derivative of SEQ ID NO:33 with one, two, three, or four amino acid modifications), and a CDRL3 having the amino acid sequence set forth in SEQ ID NO:34 (or a derivative of SEQ ID NO:34 with one, two, three, or four amino acid modifications). Examples of such antigen recognition domains having these CDRs and the ability to bind to CD64 include, without limitation, antigen recognition domains having the VH set forth in SEQ ID NO:31 and the VL set forth in SEQ ID NO:39.

In some embodiments, an antigen recognition domain of a CAR provided herein having (i) a VH having a CDRH1 having the amino acid sequence set forth in SEQ ID NO:24 (or a derivative of SEQ ID NO:24 with one, two, three, or four amino acid modifications), a CDRH2 having the amino acid sequence set forth in SEQ ID NO:25 (or a derivative of SEQ ID NO:25 with one, two, three, or four amino acid modifications), and a CDRH3 having the amino acid sequence set forth in SEQ ID NO:26 (or a derivative of SEQ ID NO:26 with one, two, three, or four amino acid modifications), and/or (ii) a VL having a CDRL1 having the amino acid sequence set forth in SEQ ID NO:32 (or a derivative of SEQ ID NO:32 with one, two, three, or four amino acid modifications), a CDRL2 having the amino acid sequence set forth in SEQ ID NO:33 (or a derivative of SEQ ID NO:33 with one, two, three, or four amino acid modifications), and a CDRL3 having the amino acid sequence set forth in SEQ ID NO:34 (or a derivative of SEQ ID NO:34 with one, two, three, or four amino acid modifications) can include any appropriate framework regions. For example, such an antigen recognition domain can include (i) a VH that includes a framework region 1 having the amino acid sequence set forth in SEQ ID NO:27 (or a derivative of SEQ ID NO:27 with one, two, three, four, five, six, seven, eight, nine, ten, or more amino acid modifications), a framework region 2 having the amino acid sequence set forth in SEQ ID NO:28 (or a derivative of SEQ ID NO:28 with one, two, three, four, five, six, seven, eight, nine, ten, or more amino acid modifications), a framework region 3 having the amino acid sequence set forth in SEQ ID NO:29 (or a derivative of SEQ ID NO:29 with one, two, three, four, five, six, seven, eight, nine, ten, or more amino acid modifications), and a framework region 4 having the amino acid sequence set forth in SEQ ID NO:30 (or a derivative of SEQ ID NO:30 with one, two, three, four, five, six, seven, eight, nine, ten, or more amino acid modifications), and/or (ii) a VL that includes a framework region 1 having the amino acid sequence set forth in SEQ ID NO:35 (or a derivative of SEQ ID NO:35 with one, two, three, four, five, six, seven, eight, nine, ten, or more amino acid modifications), a framework region 2 having the amino acid sequence set forth in SEQ ID NO:36 (or a derivative of SEQ ID NO:36 with one, two, three, four, five, six, seven, eight, nine, ten, or more amino acid modifications), a framework region 3 having the amino acid sequence set forth in SEQ ID NO:37 (or a derivative of SEQ ID NO:37 with one, two, three, four, five, six, seven, eight, nine, ten, or more amino acid modifications), and a framework region 4 having the amino acid sequence set forth in SEQ ID NO:38 (or a derivative of SEQ ID NO:38 with one, two, three, four, five, six, seven, eight, nine, ten, or more amino acid modifications).

In some embodiments, an antigen recognition domain of a CAR provided herein can include (i) a VH that includes an amino acid sequence having at least 90 percent identity to the amino acid sequence set forth in SEQ ID NO:31, and/or (ii) a VL that includes an amino acid sequence having at least 90 percent identity to the amino acid sequence set forth in SEQ ID NO:39. For example, a CAR provided herein can include (i) a VH that includes an amino acid sequence having at least 90, 91, 92, 93, 94, 95, 96, 97, 98, or 99 percent identity to the amino acid sequence set forth in SEQ ID NO:31, and/or (ii) a VL that includes an amino acid sequence having at least 90, 91, 92, 93, 94, 95, 96, 97, 98, or 99 percent identity to the amino acid sequence set forth in SEQ ID NO:39. In some cases, a CAR provided herein can include (i) a VH that includes an amino acid sequence having 100 percent identity to the amino acid sequence set forth in SEQ ID NO:31, and/or (ii) a VL that includes an amino acid sequence having 100 percent identity to the amino acid sequence set forth in SEQ ID NO:39.

In some embodiments, an antigen recognition domain of a CAR provided herein can include (i) a VH that includes an amino acid sequence having at least 90 percent identity to the amino acid sequence set forth in SEQ ID NO:31, provided that the VH includes the amino acid sequences set forth in SEQ ID NOs:24, 25, and 26, and/or (ii) a VL that includes an amino acid sequence having at least 90 percent identity to the amino acid sequence set forth in SEQ ID NO:39, provided that the VL includes the amino acid sequences set forth in SEQ ID NOs:32, 33, and 34. For example, a CAR provided herein can include (i) a VH that includes an amino acid sequence having at least 90, 91, 92, 93, 94, 95, 96, 97, 98, or 99 percent identity to the amino acid sequence set forth in SEQ ID NO:31, provided that the VH includes the amino acid sequences set forth in SEQ ID NOs:24, 25, and 26, and/or (ii) a VL that includes an amino acid sequence having at least 90, 91, 92, 93, 94, 95, 96, 97, 98, or 99 percent identity to the amino acid sequence set forth in SEQ ID NO:39, provided that the VL includes the amino acid sequences set forth in SEQ ID NOs:32, 33, and 34.

In some embodiments, an antigen recognition domain of a CAR provided herein can include (i) a VH having a CDRH1 having the amino acid sequence set forth in SEQ ID NO:40 (or a derivative of SEQ ID NO:40 with one, two, three, or four amino acid modifications), a CDRH2 having the amino acid sequence set forth in SEQ ID NO:41 (or a derivative of SEQ ID NO:41 with one, two, three, or four amino acid modifications), and a CDRH3 having the amino acid sequence set forth in SEQ ID NO:42 (or a derivative of SEQ ID NO:42 with one, two, three, or four amino acid modifications), and/or (ii) a VL having a CDRL1 having the amino acid sequence set forth in SEQ ID NO:48 (or a derivative of SEQ ID NO:48 with one, two, three, or four amino acid modifications), a CDRL2 having the amino acid sequence set forth in SEQ ID NO:49 (or a derivative of SEQ ID NO:49 with one, two, three, or four amino acid modifications), and a CDRL3 having the amino acid sequence set forth in SEQ ID NO:50 (or a derivative of SEQ ID NO:50 with one, two, three, or four amino acid modifications). Examples of such antigen recognition domains having these CDRs and the ability to bind CD64 include, without limitation, antigen recognition domains having the VH set forth in SEQ ID NO:47 and the VL set forth in SEQ ID NO:55.

In some embodiments, an antigen recognition domain of a CAR provided herein having (i) a VH having a CDRH1 having the amino acid sequence set forth in SEQ ID NO:40 (or a derivative of SEQ ID NO:40 with one, two, three, or four amino acid modifications), a CDRH2 having the amino acid sequence set forth in SEQ ID NO:41 (or a derivative of SEQ ID NO:41 with one, two, three, or four amino acid modifications), and a CDRH3 having the amino acid sequence set forth in SEQ ID NO:42 (or a derivative of SEQ ID NO:42 with one, two, three, or four amino acid modifications), and/or (ii) a VL having a CDRL1 having the amino acid sequence set forth in SEQ ID NO:48 (or a derivative of SEQ ID NO:48 with one, two, three, or four amino acid modifications), a CDRL2 having the amino acid sequence set forth in SEQ ID NO:49 (or a derivative of SEQ ID NO:49 with one, two, three, or four amino acid modifications), and a CDRL3 having the amino acid sequence set forth in SEQ ID NO:50 (or a derivative of SEQ ID NO:50 with one, two, three, or four amino acid modifications) can include any appropriate framework regions. For example, such an antigen recognition domain can include (i) a VH that includes a framework region 1 having the amino acid sequence set forth in SEQ ID NO:43 (or a derivative of SEQ ID NO:43 with one, two, three, four, five, six, seven, eight, nine, ten, or more amino acid modifications), a framework region 2 having the amino acid sequence set forth in SEQ ID NO:44 (or a derivative of SEQ ID NO:44 with one, two, three, four, five, six, seven, eight, nine, ten, or more amino acid modifications), a framework region 3 having the amino acid sequence set forth in SEQ ID NO:45 (or a derivative of SEQ ID NO:45 with one, two, three, four, five, six, seven, eight, nine, ten, or more amino acid modifications), and a framework region 4 having the amino acid sequence set forth in SEQ ID NO:46 (or a derivative of SEQ ID NO:46 with one, two, three, four, five, six, seven, eight, nine, ten, or more amino acid modifications), and/or (ii) a VL that includes a framework region 1 having the amino acid sequence set forth in SEQ ID NO:51 (or a derivative of SEQ ID NO:51 with one, two, three, four, five, six, seven, eight, nine, ten, or more amino acid modifications), a framework region 2 having the amino acid sequence set forth in SEQ ID NO:52 (or a derivative of SEQ ID NO:52 with one, two, three, four, five, six, seven, eight, nine, ten, or more amino acid modifications), a framework region 3 having the amino acid sequence set forth in SEQ ID NO:53 (or a derivative of SEQ ID NO:53 with one, two, three, four, five, six, seven, eight, nine, ten, or more amino acid modifications), and a framework region 4 having the amino acid sequence set forth in SEQ ID NO:54 (or a derivative of SEQ ID NO:54 with one, two, three, four, five, six, seven, eight, nine, ten, or more amino acid modifications).

In some embodiments, an antigen recognition domain of a CAR provided herein can include (i) a VH that includes an amino acid sequence having at least 90 percent identity to the amino acid sequence set forth in SEQ ID NO:47, and/or (ii) a VL that includes an amino acid sequence having at least 90 percent identity to the amino acid sequence set forth in SEQ ID NO:55. For example, a CAR provided herein can include (i) a VH that includes an amino acid sequence having at least 90, 91, 92, 93, 94, 95, 96, 97, 98, or 99 percent identity to the amino acid sequence set forth in SEQ ID NO:47, and/or (ii) a VL that includes an amino acid sequence having at least 90, 91, 92, 93, 94, 95, 96, 97, 98, or 99 percent identity to the amino acid sequence set forth in SEQ ID NO:55. In some cases, a CAR provided herein can include (i) a VH that includes an amino acid sequence having 100 percent identity to the amino acid sequence set forth in SEQ ID NO:47, and/or (ii) a VL that includes an amino acid sequence having 100 percent identity to the amino acid sequence set forth in SEQ ID NO:55.

In some embodiments, an antigen recognition domain of a CAR provided herein can include (i) a VH that includes an amino acid sequence having at least 90 percent identity to the amino acid sequence set forth in SEQ ID NO:47, provided that the VH includes the amino acid sequences set forth in SEQ ID NOs:40, 41, and 42, and/or (ii) a VL that includes an amino acid sequence having at least 90 percent identity to the amino acid sequence set forth in SEQ ID NO:55, provided that the VL includes the amino acid sequences set forth in SEQ ID NOs:48, 49, and 50. For example, a CAR provided herein can include (i) a VH that includes an amino acid sequence having at least 90, 91, 92, 93, 94, 95, 96, 97, 98, or 99 percent identity to the amino acid sequence set forth in SEQ ID NO:47, provided that the VH includes the amino acid sequences set forth in SEQ ID NOs:40, 41, and 42, and/or (ii) a VL that includes an amino acid sequence having at least 90, 91, 92, 93, 94, 95, 96, 97, 98, or 99 percent identity to the amino acid sequence set forth in SEQ ID NO:55, provided that the VL includes the amino acid sequences set forth in SEQ ID NOs:48, 49, and 50.

In some embodiments, an antigen recognition domain of a CAR provided herein can include (a) a VH, wherein the VH comprises (i) a CDRH1 that comprises, consists essentially of, or consists of the amino acid sequence set forth in SEQ ID NO:24, (ii) a CDRH2 that comprises, consists essentially of, or consists of the amino acid sequence set forth in SEQ ID NO:25, and (iii) a CDRH3 that comprises, consists essentially of, or consists of the amino acid sequence set forth in SEQ ID NO:26, and/or (b) a VL, wherein the VL comprises (i) a CDRL1 that comprises, consists essentially of, or consists of the amino acid sequence set forth in SEQ ID NO:32, (ii) a CDRL2 that comprises, consists essentially of, or consists of the amino acid sequence set forth in SEQ ID NO:33, and (iii) a CDRL3 that comprises, consists essentially of, or consists of the amino acid sequence set forth in SEQ ID NO:34. As used herein, a “CDRH1 that consists essentially of the amino acid sequence set forth in SEQ ID NO:24” is a CDRH1 that has zero, one, or two amino acid substitutions within SEQ ID NO:24, that has zero, one, two, three, four, or five amino acid residues directly preceding SEQ ID NO:24, and/or that has zero, one, two, three, four, or five amino acid residues directly following SEQ ID NO:24, provided that the CAR maintains its basic ability to bind to CD64. Examples of a CDRH1 that consists essentially of the amino acid sequence set forth in SEQ ID NO:24 include, without limitation, those set forth in Table 2.

TABLE 2
Exemplary CDRHIs that consist essentially
of the amino acid sequence set forth
in SEQ ID NO: 24.

| Sequence | SEQ ID NO: |
|---|---|
| TGFIFSGYG | 56 |
| CGFIFSGYG | 57 |
| GGFIFSGYG | 58 |
| SSFIFSGYG | 59 |
| SCFIFSGYG | 60 |
| SQFIFSGYG | 61 |
| SNFIFSGYG | 62 |
| STFIFSGYG | 63 |
| SGWIFSGYG | 64 |
| SGFIWSGYG | 65 |
| SGWIWSGYG | 66 |
| SGPIFSGYG | 67 |
| SGFIPSGYG | 68 |
| SGPIPSGYG | 69 |
| SGYIFSGYG | 70 |
| SGFIYSGYG | 71 |
| SGYIYSGYG | 72 |
| SGFLFSGYG | 73 |
| SGFVFSGYG | 74 |
| SGFAFSGYG | 75 |
| SGFMFSGYG | 76 |
| SGFIFTGYG | 77 |
| SGFIFCGYG | 78 |
| SGFIFGGYG | 79 |
| SGFIFSSYG | 80 |
| SGFIFSCYG | 81 |
| SGFIFSTYG | 82 |
| SGFIFSQYG | 83 |
| SGFIFSNYG | 84 |
| SGFIFSGYS | 85 |
| SGFIFSGYC | 86 |
| SGFIFSGYT | 87 |
| SGFIFSGYQ | 88 |
| SGFIFSGYN | 89 |
| SGFIFSGFG | 90 |
| SGFIFSGSG | 91 |
| SGFIFSGTG | 92 |

As used herein, a “CDRH2 that consists essentially of the amino acid sequence set forth in SEQ ID NO:25” is a CDRH2 that has zero, one, or two amino acid substitutions within SEQ ID NO:25, that has zero, one, two, three, four, or five amino acid residues directly preceding SEQ ID NO:25, and/or that has zero, one, two, three, four, or five amino acid residues directly following SEQ ID NO:25, provided that the CAR maintains its basic ability to bind to CD64. Examples of a CDRH2 that consists essentially of the amino acid sequence set forth in SEQ ID NO:25 include, without limitation, those set forth in Table 3.

TABLE 3
Exemplary CDRH2s that consist essentially
of the amino acid sequence set forth
in SEQ ID NO: 25.

| Sequence | SEQ ID NO: |
|---|---|
| LWYDGSNK | 93 |
| VWYDGSNK | 94 |
| AWYDGSNK | 95 |
| IFYDGSNK | 96 |
| IPYDGSNK | 97 |
| IHYDGSNK | 98 |
| IWFDGSNK | 99 |
| IWSDGSNK | 100 |
| IWTDGSNK | 101 |
| IWYEGSNK | 102 |
| IWYNGSNK | 103 |
| IWYDSSNK | 104 |
| IWYDTSNK | 105 |
| IWYDCSNK | 106 |
| IWYDNSNK | 107 |
| IWYDQSNK | 108 |
| IWYDGCNK | 109 |
| IWYDGTNK | 110 |
| IWYDGGNK | 111 |
| IWYDGNNK | 112 |
| IWYDGQNK | 113 |
| IWYDGSQK | 114 |
| IWYDGSGK | 115 |
| IWYDGSSK | 116 |
| IWYDGSCK | 117 |
| IWYDGSTK | 118 |
| IWYDGSNR | 119 |
| IWYDGSNH | 120 |

As used herein, a “CDRH3 that consists essentially of the amino acid sequence set forth in SEQ ID NO:26” is a CDRH3 that has zero, one, or two amino acid substitutions within SEQ ID NO:26, that has zero, one, two, three, four, or five amino acid residues directly preceding SEQ ID NO:26, and/or that has zero, one, two, three, four, or five amino acid residues directly following SEQ ID NO:26, provided that the CAR maintains its basic ability to bind to CD64. Examples of a CDRH3 that consists essentially of the amino acid sequence set forth in SEQ ID NO:26 include, without limitation, those set forth in Table 4.

TABLE 4
Exemplary CDRH3s that consist essentially
of the amino acid sequence set forth
in SEQ ID NO: 26.

| Sequence | SEQ ID NO: |
|---|---|
| VRDTGDRFFDY | 121 |
| LRDTGDRFFDY | 122 |
| IRDTGDRFFDY | 123 |
| AKDTGDRFFDY | 124 |
| AHDTGDRFFDY | 125 |
| ARETGDRFFDY | 126 |
| ARDSGDRFFDY | 127 |
| ARDCGDRFFDY | 128 |
| ARDYGDRFFDY | 129 |
| ARDTQDRFFDY | 130 |
| ARDTCDRFFDY | 131 |
| ARDTTDRFFDY | 132 |
| ARDTSDRFFDY | 133 |
| ARDTGERFFDY | 134 |
| ARDTGDKFFDY | 135 |
| ARDTGDHFFDY | 136 |
| ARDTGDRWFDY | 137 |
| ARDTGDRPFDY | 138 |
| ARDTGDRYFDY | 139 |
| ARDTGDRFWDY | 140 |
| ARDTGDRFPDY | 141 |
| ARDTGDRFYDY | 142 |
| ARDTGDRFFEY | 143 |
| ARDTGDRFFDT | 144 |
| ARDTGDRFFDS | 145 |
| ARDTGDRFFDF | 146 |

As used herein, a “CDRL1 that consists essentially of the amino acid sequence set forth in SEQ ID NO:32” is a CDRL1 that has zero, one, or two amino acid substitutions within SEQ ID NO:32, that has zero, one, two, three, four, or five amino acid residues directly preceding SEQ ID NO:32, and/or that has zero, one, two, three, four, or five amino acid residues directly following SEQ ID NO:32, provided that the CAR maintains its basic ability to bind to CD64. Examples of a CDRL1 that consists essentially of the amino acid sequence set forth in SEQ ID NO:32 include, without limitation, those set forth in Table 5.

TABLE 5
Exemplary CDRL1s that consist essentially of
the amino acid sequence set forth
in SEQ ID NO: 32.

| Sequence | SEQ ID NO: |
|---|---|
| GSVSSY | 147 |
| NSVSSY | 148 |
| QTVSSY | 149 |
| QCVSSY | 150 |
| QYVSSY | 151 |
| QSASSY | 152 |
| QSLSSY | 153 |
| QSISSY | 154 |
| QSMSSY | 155 |
| QSVTSY | 156 |
| QSVCSY | 157 |
| QSVYSY | 158 |
| QSVSTY | 159 |
| QSVSCY | 160 |
| QSVSYY | 161 |
| QSVSSS | 162 |
| QSVSST | 163 |
| QSVSSF | 164 |

As used herein, a “CDRL2 that consists essentially of the amino acid sequence set forth in SEQ ID NO:33” is a CDRL2 that has zero, one, or two amino acid substitutions within SEQ ID NO:33, that has zero, one, two, three, four, or five amino acid residues directly preceding SEQ ID NO:33, and/or that has zero, one, two, three, four, or five amino acid residues directly following SEQ ID NO:33, provided that the CAR maintains its basic ability to bind to CD64. Examples of a CDRL2 that consists essentially of the amino acid sequence set forth in SEQ ID NO:33 include, without limitation, those set forth in Table 6.

TABLE 6
Exemplary CDRL2s that consist essentially of
the amino acid sequence set forth in
SEQ ID NO: 33.

| Sequence | SEQ ID NO: |
|---|---|
| EAS | 165 |
| DVS | 166 |
| DLS | 167 |
| DIS | 168 |
| DMS | 169 |
| DAT | 170 |
| DAC | 171 |
| DAY | 172 |

As used herein, a “CDRL3 that consists essentially of the amino acid sequence set forth in SEQ ID NO:34” is a CDRL3 that has zero, one, or two amino acid substitutions within SEQ ID NO:34, that has zero, one, two, three, four, or five amino acid residues directly preceding SEQ ID NO:34, and/or that has zero, one, two, three, four, or five amino acid residues directly following SEQ ID NO:34, provided that the CAR maintains its basic ability to bind to CD64. Examples of a CDRL3 that consists essentially of the amino acid sequence set forth in SEQ ID NO:34 include, without limitation, those set forth in Table 7.

TABLE 7
Exemplary CDRL3s that consist essentially
of the amino acid sequence set forth
in SEQ ID NO: 34.

| Sequence | SEQ ID NO: |
|---|---|
| NLRSNWPPYT | 173 |
| PLRSNWPPYT | 174 |
| QIRSNWPPYT | 175 |
| QVRSNWPPYT | 176 |
| QARSNWPPYT | 177 |
| QLKSNWPPYT | 178 |
| QLHSNWPPYT | 179 |
| QLRTNWPPYT | 180 |
| QLRCNWPPYT | 181 |
| QLRYNWPPYT | 182 |
| QLRSQWPPYT | 183 |
| QLRSNFPPYT | 184 |
| QLRSNYPPYT | 185 |
| QLRSNPPPYT | 186 |
| QLRSNWFPYT | 187 |
| QLRSNWQPYT | 188 |
| QLRSNWNPYT | 189 |
| QLRSNWPWYT | 190 |
| QLRSNWWPYT | 191 |
| QLRSNWPNYT | 192 |
| QLRSNWPQYT | 193 |
| QLRSNWPPTT | 194 |
| QLRSNWPPST | 195 |
| QLRSNWPPFT | 196 |
| QLRSNWPPYS | 197 |
| QLRSNWPPYC | 198 |
| QLRSNWPPYY | 199 |

In some embodiments, an antigen recognition domain of a CAR provided herein can include (a) a VH, wherein the VH comprises (i) a CDRH1 that comprises, consists essentially of, or consists of the amino acid sequence set forth in SEQ ID NO:40, (ii) a CDRH2 that comprises, consists essentially of, or consists of the amino acid sequence set forth in SEQ ID NO:41, and (iii) a CDRH3 that comprises, consists essentially of, or consists of the amino acid sequence set forth in SEQ ID NO:42, and/or (b) a VL, wherein the VL comprises (i) a CDRL1 that comprises, consists essentially of, or consists of the amino acid sequence set forth in SEQ ID NO:48, (ii) a CDRL2 that comprises, consists essentially of, or consists of the amino acid sequence set forth in SEQ ID NO:49, and (iii) a CDRL3 that comprises, consists essentially of, or consists of the amino acid sequence set forth in SEQ ID NO:50. As used herein, a “CDRH1 that consists essentially of the amino acid sequence set forth in SEQ ID NO:40” is a CDRH1 that has zero, one, or two amino acid substitutions within SEQ ID NO:40, that has zero, one, two, three, four, or five amino acid residues directly preceding SEQ ID NO:40, and/or that has zero, one, two, three, four, or five amino acid residues directly following SEQ ID NO:40, provided that the CAR maintains its basic ability to bind to CD64. Examples of a CDRH1 that consists essentially of the amino acid sequence set forth in SEQ ID NO:40 include, without limitation, those set forth in Table 8.

TABLE 8
Exemplary CDRHIs that consist essentially
of the amino acid sequence set forth
in SEQ ID NO: 40.

| Sequence | SEQ ID NO: |
|---|---|
| SYGMH | 200 |
| CYGMH | 201 |
| TYGMH | 202 |
| QYGMH | 203 |
| NYGMH | 204 |
| GYSMH | 205 |
| GYCMH | 206 |
| GYTMH | 207 |
| GYQMH | 208 |
| GYNMH | 209 |
| GFGMH | 210 |
| GSGMH | 211 |
| GTGMH | 212 |
| GYGIH | 213 |
| GYGLH | 214 |
| GYGVH | 215 |
| GYGAH | 216 |
| GYGCH | 217 |
| GYGMK | 218 |
| GYGMR | 219 |
| GYGMP | 220 |

As used herein, a “CDRH2 that consists essentially of the amino acid sequence set forth in SEQ ID NO:41” is a CDRH2 that has zero, one, or two amino acid substitutions within SEQ ID NO:41, that has zero, one, two, three, four, or five amino acid residues directly preceding SEQ ID NO:41, and/or that has zero, one, two, three, four, or five amino acid residues directly following SEQ ID NO:41, provided that the CAR maintains its basic ability to bind to CD64. Examples of a CDRH2 that consists essentially of the amino acid sequence set forth in SEQ ID NO:41 include, without limitation, those set forth in Table 9.

TABLE 9
Exemplary CDRH2s that consist essentially
of the amino acid sequence set forth
in SEQ ID NO: 41.

| Sequence | SEQ ID NO: |
|---|---|
| LIWYDGSNKYYADSVKG | 221 |
| IIWYDGSNKYYADSVKG | 222 |
| AIWYDGSNKYYADSVKG | 223 |
| MIWYDGSNKYYADSVKG | 224 |
| VLWYDGSNKYYADSVKG | 225 |
| VVWYDGSNKYYADSVKG | 226 |
| VAWYDGSNKYYADSVKG | 227 |
| VMWYDGSNKYYADSVKG | 228 |
| VILWYDGSNKYYADSVKG | 229 |
| VIVWYDGSNKYYADSVKG | 230 |
| VIAWYDGSNKYYADSVKG | 231 |
| VIIFYDGSNKYYADSVKG | 232 |
| VIIPYDGSNKYYADSVKG | 233 |
| VIIHYDGSNKYYADSVKG | 234 |
| VIIWFDGSNKYYADSVKG | 235 |
| VIIWSDGSNKYYADSVKG | 236 |
| VIIWTDGSNKYYADSVKG | 237 |
| VIIWYEGSNKYYADSVKG | 238 |
| VIIWYNGSNKYYADSVKG | 239 |
| VIIWYDSSNKYYADSVKG | 240 |
| VIIWYDTSNKYYADSVKG | 241 |
| VIIWYDCSNKYYADSVKG | 242 |
| VIIWYDNSNKYYADSVKG | 243 |
| VIIWYDQSNKYYADSVKG | 244 |
| VIIWYDGCNKYYADSVKG | 245 |
| VIIWYDGTNKYYADSVKG | 246 |
| VIIWYDGGNKYYADSVKG | 247 |
| VIIWYDGNNKYYADSVKG | 248 |
| VIIWYDGQNKYYADSVKG | 249 |
| VIIWYDGSQKYYADSVKG | 250 |
| VIIWYDGSGKYYADSVKG | 251 |
| VIIWYDGSSKYYADSVKG | 252 |
| VIIWYDGSCKYYADSVKG | 253 |
| VIIWYDGSTKYYADSVKG | 254 |
| VIIWYDGSNRYYADSVKG | 255 |
| VIIWYDGSNHYYADSVKG | 256 |
| VIWYDGSNKSYADSVKG | 257 |
| VIWYDGSNKTYADSVKG | 258 |
| VIWYDGSNKFYADSVKG | 259 |
| VIWYDGSNKYSADSVKG | 260 |
| VIWYDGSNKYTADSVKG | 261 |
| VIWYDGSNKYFADSVKG | 262 |
| VIWYDGSNKYYVDSVKG | 263 |
| VIWYDGSNKYYLDSVKG | 264 |
| VIWYDGSNKYYIDSVKG | 265 |
| VIWYDGSNKYYMDSVKG | 266 |
| VIWYDGSNKYYAESVKG | 267 |
| VIWYDGSNKYYADCVKG | 268 |
| VIWYDGSNKYYADTVKG | 269 |
| VIWYDGSNKYYADSAKG | 270 |
| VIWYDGSNKYYADSLKG | 271 |
| VIWYDGSNKYYADSIKG | 272 |
| VIWYDGSNKYYADSMKG | 273 |
| VIWYDGSNKYYADSVRG | 274 |
| VIWYDGSNKYYADSVHG | 275 |
| VIWYDGSNKYYADSVKS | 276 |
| VIWYDGSNKYYADSVKT | 277 |
| VIWYDGSNKYYADSVKC | 278 |

As used herein, a “CDRH3 that consists essentially of the amino acid sequence set forth in SEQ ID NO:42” is a CDRH3 that has zero, one, or two amino acid substitutions within SEQ ID NO:42, that has zero, one, two, three, four, or five amino acid residues directly preceding SEQ ID NO:42, and/or that has zero, one, two, three, four, or five amino acid residues directly following SEQ ID NO:42, provided that the CAR maintains its basic ability to bind to CD64. Examples of a CDRH3 that consists essentially of the amino acid sequence set forth in SEQ ID NO:42 include, without limitation, those set forth in Table 10.

TABLE 10
Exemplary CDRH3s that consist essentially
of the amino acid sequence set
forth in SEQ ID NO: 42.

| Sequence | SEQ ID NO: |
|---|---|
| ETGDRFFDY | 279 |
| DSGDRFFDY | 280 |
| DCGDRFFDY | 281 |
| DYGDRFFDY | 282 |
| DTQDRFFDY | 283 |
| DTCDRFFDY | 284 |
| DTTDRFFDY | 285 |
| DTSDRFFDY | 286 |
| DTGERFFDY | 287 |
| DTGDKFFDY | 288 |
| DTGDHFFDY | 289 |
| DTGDRWFDY | 290 |
| DTGDRPFDY | 291 |
| DTGDRYFDY | 292 |
| DTGDRFWDY | 293 |
| DTGDRFPDY | 294 |
| DTGDRFYDY | 295 |
| DTGDRFFEY | 296 |
| DTGDRFFDT | 297 |
| DTGDRFFDS | 298 |
| DTGDRFFDF | 299 |

As used herein, a “CDRL1 that consists essentially of the amino acid sequence set forth in SEQ ID NO:48” is a CDRL1 that has zero, one, or two amino acid substitutions within SEQ ID NO:48, that has zero, one, two, three, four, or five amino acid residues directly preceding SEQ ID NO:48, and/or that has zero, one, two, three, four, or five amino acid residues directly following SEQ ID NO:48, provided that the CAR maintains its basic ability to bind to CD64. Examples of a CDRL1 that consists essentially of the amino acid sequence set forth in SEQ ID NO:48 include, without limitation, those set forth in Table 11.

TABLE 11
Exemplary CDRL1s that consist essentially
of the amino acid sequence set
forth in SEQ ID NO: 48.

| Sequence | SEQ ID NO: |
|---|---|
| KASQSVSSYLA | 300 |
| HASQSVSSYLA | 301 |
| RVSQSVSSYLA | 302 |
| RLSQSVSSYLA | 303 |
| RISQSVSSYLA | 304 |
| RMSQSVSSYLA | 305 |
| RATQSVSSYLA | 306 |
| RACQSVSSYLA | 307 |
| RAYQSVSSYLA | 308 |
| RASGSVSSYLA | 309 |
| RASNSVSSYLA | 310 |
| RASQTVSSYLA | 311 |
| RASQCVSSYLA | 312 |
| RASQYVSSYLA | 313 |
| RASQSASSYLA | 314 |
| RASQSLSSYLA | 315 |
| RASQSISSYLA | 316 |
| RASQSMSSYLA | 317 |
| RASQSVTSYLA | 318 |
| RASQSVCSYLA | 319 |
| RASQSVYSYLA | 320 |
| RASQSVSTYLA | 321 |
| RASQSVSCYLA | 322 |
| RASQSVSYYLA | 323 |
| RASQSVSSSLA | 324 |
| RASQSVSSTLA | 325 |
| RASQSVSSFLA | 326 |
| RASQSVSSYIA | 327 |
| RASQSVSSYVA | 328 |
| RASQSVSSYAA | 329 |
| RASQSVSSYMA | 330 |
| RASQSVSSYLV | 331 |
| RASQSVSSYLL | 332 |
| RASQSVSSYLI | 333 |
| RASQSVSSYLM | 334 |

As used herein, a “CDRL2 that consists essentially of the amino acid sequence set forth in SEQ ID NO:49” is a CDRL2 that has zero, one, or two amino acid substitutions within SEQ ID NO:49, that has zero, one, two, three, four, or five amino acid residues directly preceding SEQ ID NO:49, and/or that has zero, one, two, three, four, or five amino acid residues directly following SEQ ID NO:49, provided that the CAR maintains its basic ability to bind to CD64. Examples of a CDRL2 that consists essentially of the amino acid sequence set forth in SEQ ID NO:49 include, without limitation, those set forth in Table 12.

TABLE 12
Exemplary CDRL2s that consist essentially
of the amino acid sequence set
forth in SEQ ID NO: 49.

| Sequence | SEQ ID NO: |
|---|---|
| EASSRAT | 335 |
| DVSSRAT | 336 |
| DLSSRAT | 337 |
| DISSRAT | 338 |
| DMSSRAT | 339 |
| DATSRAT | 340 |
| DACSRAT | 341 |
| DAYSRAT | 342 |
| DASTRAT | 343 |
| DASCRAT | 344 |
| DASYRAT | 345 |
| DASSKAT | 346 |
| DASSHAT | 347 |
| DASSRVT | 348 |
| DASSRLT | 349 |
| DASSRIT | 350 |
| DASSRAS | 351 |
| DASSRAC | 352 |
| DASSRAY | 353 |

As used herein, a “CDRL3 that consists essentially of the amino acid sequence set forth in SEQ ID NO:50” is a CDR3 that has zero, one, or two amino acid substitutions within SEQ ID NO:50, that has zero, one, two, three, four, or five amino acid residues directly preceding SEQ ID NO:50, and/or that has zero, one, two, three, four, or five amino acid residues directly following SEQ ID NO:50, provided that the CAR maintains its basic ability to bind to CD64. Examples of a CDRL3 that consists essentially of the amino acid sequence set forth in SEQ ID NO:50 include, without limitation, those set forth in Table 13.

TABLE 13
Exemplary CDR3s that consist essentially
of the amino acid sequence set forth
in SEQ ID NO: 50.

| Sequence | SEQ ID NO: |
|---|---|
| NLRSNWPPYT | 354 |
| PLRSNWPPYT | 355 |
| QIRSNWPPYT | 356 |
| QVRSNWPPYT | 357 |
| QARSNWPPYT | 358 |
| QLKSNWPPYT | 359 |
| QLHSNWPPYT | 360 |
| QLRTNWPPYT | 361 |
| QLRCNWPPYT | 362 |
| QLRYNWPPYT | 363 |
| QLRSQWPPYT | 364 |
| QLRSNFPPYT | 365 |
| QLRSNYPPYT | 366 |
| QLRSNPPPYT | 367 |
| QLRSNWFPYT | 368 |
| QLRSNWQPYT | 369 |
| QLRSNWNPYT | 370 |
| QLRSNWPWYT | 371 |
| QLRSNWWPYT | 372 |
| QLRSNWPNYT | 373 |
| QLRSNWPQYT | 374 |
| QLRSNWPPTT | 375 |
| QLRSNWPPST | 376 |
| QLRSNWPPFT | 377 |
| QLRSNWPPYS | 378 |
| QLRSNWPPYC | 379 |
| QLRSNWPPYY | 380 |

A representative number of antigen recognition domains of the CARs provided herein are further described in Table 14.

TABLE 14
Representative antigen recognition domains

| | SEQ ID NO(s) of: | | | | | |
|---|---|---|---|---|---|---|
| | | VH domain | | | VL domain | |
| Clone # | CDRHs 1, | Framework | VH | CDRLs 1, | Framework | VL |
| (binder type) | 2, and 3 | Regions | domain | 2, and 3 | Regions | domain |
| #1 (scFv) | 24, 25, 26 | 27, 28, 29, 30 | 31 | 32, 33, 34 | 35, 36, 37, 38 | 39 |
| #2 (scFv) | 40, 41, 42 | 43, 44, 45, 46 | 47 | 48, 49, 50 | 51, 52, 53, 54 | 55 |

### Signal Peptides

In some embodiments, any of the CARs provided herein can further comprise a signal peptide (also known in the art as a signal peptide, signal sequence, signal peptide sequence, leader peptide, and leader peptide sequence). In some embodiments, the first domain of the CAR described herein comprises a signal peptide or a leader peptide sequence. Exemplary signal sequences include but are not limited to a CD8α signal sequence or an IgG signal sequence. In some embodiments, the CAR described herein does not comprise a signal peptide.

In some embodiments, the CAR (e.g., the first domain of the CAR) may comprise a human CD8α signal sequence.

In some embodiments, the CAR (e.g., the first recognition domain of the CAR) may comprise a human IgG signal sequence. In some aspects, the hinge domain of a CAR of the present disclosure can comprise, consist essentially of, or consist of an amino acid sequence having at least 65%, 70%, 75%, 80%, 85%, 90%, 95%, 96%, 97%, 98%, 99%, or 100% (or any percentage in between) identical to SEQ ID NO: 23.

### Hinge Domains

In some embodiments, a hinge domain (also known in the art as a spacer region or a stalk region) is located between the antigen recognition domain and the transmembrane domain of the CAR. In particular, stalk regions are used to provide more flexibility and accessibility for the extracellular antigen recognition domain. In some embodiments, a hinge domain may comprise up to about 300 amino acids. In some embodiments, the hinge comprises about 10 to about 100 amino acids in length. In some embodiments, the hinge comprises about 25 to about 50 amino acids in length. In some embodiments, the hinge domain establishes an optimal effector-target inter membrane distance. In some embodiments, the hinge domain provides flexibility for antigen recognition domain to bind the target antigen. Any protein that is stable and/or dimerizes can serve this purpose.

A hinge domain may be derived from all or part of naturally occurring molecules, such as from all or part of the extracellular region of CD8α or CD28, or from all or part of an antibody heavy-chain constant region. Alternatively, the hinge domain may be a synthetic sequence that corresponds to a naturally occurring hinge sequence or may be an entirely synthetic hinge sequence.

In some embodiments, the hinge domain is a part of human CD8α chain (e.g., NP_001139345.1). In some embodiments, the hinge domain of CARs described herein comprises a subsequence of CD8α or CD28 either in wild-type form or mutated to avoid Fc-receptor binding in particular the hinge domain of any of an CD8α, or a CD28. In some embodiments, the stalk region comprises a human CD8α hinge or a human CD28 hinge.

In some embodiments, the hinge domain of a CAR of the present disclosure can comprise, consist essentially of, or consist of an amino acid sequence having at least 65%, 70%, 75%, 80%, 85%, 90%, 95%, 96%, 97%, 98%, 99%, or 100% (or any percentage in between) identical to SEQ ID NO: 4.

In some embodiments, the hinge domain of a CAR of the present disclosure can comprise, consist essentially of, or consist of an amino acid sequence having at least 65%, 70%, 75%, 80%, 85%, 90%, 95%, 96%, 97%, 98%, 99%, or 100% (or any percentage in between) identical to SEQ ID NO: 8.

### Transmembrane Domains

Suitable transmembrane domains for a CAR disclosed herein have the ability to (a) be expressed at the surface of a cell, which is in some embodiments an immune cell such as, for example a T cell, and/or (b) interact with the ligand-binding domain and intracellular signaling domain for directing cellular response of an immune cell against a predefined target cell. The transmembrane domain can be derived either from a natural or from a synthetic source. The transmembrane domain can be derived from any membrane-bound or transmembrane protein. The transmembrane domains can include the transmembrane region(s) of CD8α and/or CD28. In some embodiments, the transmembrane domain comprises a CD8α transmembrane domain. In some embodiments, the transmembrane domain comprises a CD28 transmembrane domain.

Alternatively, the transmembrane domain can be synthetic, and can comprise hydrophobic residues such as leucine and valine. In some embodiments, a triplet of phenylalanine, tryptophan and valine is found at one or both termini of a synthetic transmembrane domain. Optionally, a short oligonucleotide or polypeptide linker, in some embodiments, between 2 and 10 amino acids in length may form the linkage between the transmembrane domain and the intracellular domain of a CAR. In some embodiments, the linker is a glycine-serine linker.

In some embodiments, the transmembrane domain of a CAR of the present disclosure can comprise, consist essentially of, or consist of an amino acid sequence having at least 65%, 70%, 75%, 80%, 85%, 90%, 95%, 96%, 97%, 98%, 99%, or 100% (or any percentage in between) identical to SEQ ID NO: 5.

In some embodiments, the transmembrane domain of a CAR of the present disclosure can comprise, consist essentially of, or consist of an amino acid sequence having at least 65%, 70%, 75%, 80%, 85%, 90%, 95%, 96%, 97%, 98%, 99%, or 100% (or any percentage in between) identical to SEQ ID NO: 9.

### Costimulatory Domains

The intracellular domain of a CAR provided herein may comprise one or more costimulatory domains. The costimulatory domains can include a 4-1BB (CD137) and/or a CD28 costimulatory domain, or a fragment thereof, or a combination thereof. In some embodiments, a CAR described herein comprises a CD28 costimulatory domain or a fragment thereof. In some embodiments, a CAR described herein comprises a 4-1BB (CD137) costimulatory domain or a fragment thereof.

In some embodiments, the costimulatory domain of a CAR of the present disclosure can comprise, consist essentially of, or consist of an amino acid sequence having at least 65%, 70%, 75%, 80%, 85%, 90%, 95%, 96%, 97%, 98%, 99%, or 100% (or any percentage in between) identical to SEQ ID NO: 6.

In some embodiments, the costimulatory domain of a CAR of the present disclosure can comprise, consist essentially of, or consist of an amino acid sequence having at least 65%, 70%, 75%, 80%, 85%, 90%, 95%, 96%, 97%, 98%, 99%, or 100% (or any percentage in between) identical to SEQ ID NO: 10.

### Activation Domains

In some embodiments, the activation domain of a CAR disclosed herein is responsible for activation of at least one of the normal effector functions of the immune cell (e.g. T cell) in which the CAR is expressed. The terms “intracellular signaling domain” or “intracellular domain” are used interchangeably and refer to a domain that comprises a co-stimulatory domain and/or an activation domain. The term “effector function” refers to a specialized function of a cell. Effector function of a T-cell, for example, may be cytolytic activity or helper activity including the secretion of cytokines. The term “activation domain” refers to the portion of a protein which transduces the effector function signal and directs the cell to perform a specialized function. While usually an entire activation domain can be employed, in many cases it is not necessary to use the entire chain. To the extent that a truncated portion of the activation domain is used, such truncated portion may be used in place of the intact chain as long as it transduces the effector function signal. The term activation domain is thus meant to include any truncated portion of the activation domain sufficient to transduce the effector function signal. In some embodiments, the activation domain further comprises a signaling domain for T-cell activation. In some instances, the signaling domain for T-cell activation comprises an intracellular domain derived from CD3ζ (CD3zeta; CD3z). In some embodiments, the CAR described herein comprises at least one (e.g., one, two, three, or more) activation domains selected from a CD3ζ or a fragment thereof. In some embodiments, the CAR described herein has an activation domain comprising a domain derived from CD3ζ (CD3zeta; CD3z).

In some embodiments, the activation domain of a CAR of the present disclosure can comprise, consist essentially of, or consist of an amino acid sequence having at least 65%, 70%, 75%, 80%, 85%, 90%, 95%, 96%, 97%, 98%, 99%, or 100% (or any percentage in between) identical to SEQ ID NO: 7.

In some embodiments, the CD3zeta activation domain comprises a mutation in an ITAM domain. Examples of mutations in ITAM domains of CD3zeta are provided in Feucht et al., Nat Med. 2019; 25(1): 82-88. In some embodiments, each of the two tyrosine residues in one or more of ITAM1, ITAM2, or ITAM3 domains of the CD3zeta activation domain are point-mutated to a phenylalanine residue. In some embodiments, the CD3zeta activation domain comprises a deletion of one or more of the ITAM1, ITAM2, or ITAM3 domains.

Table 15 provides exemplary amino acid sequences of the domains which can be used in the CARs described herein. In some embodiments, a CAR provided herein comprises one or more domains described in Table 15, or a fragment or portion thereof.

TABLE 15
Exemplary Amino Acid Sequences of CAR Domains

| Exemplary CAR | | SEQ ID |
|---|---|---|
| domains | Amino Acid Sequence | NO: |
| SIGNAL PEPTIDE | | |
| human CD8alpha signal | MALPVTALLLPLALLLHAARP | 23 |
| sequence | | |
| HINGES | | |
| human CD8alpha hinge | TTTPAPRPPTPAPTIASQPLSLRPEACRP | 4 |
| domain | AAGGAVHTRGLDFACD | |
| human CD28 hinge | IEVMYPPPYLDNEKSNGTIIHVKGKHLCP | 8 |
| domain | SPLFPGPSKP | |
| TRANSMEMBRANE | | |
| human CD8alpha | IYIWAPLAGTCGVLLLSLVITLYC | 5 |
| transmembrane domain | | |
| human CD28 | FWVLVVVGGVLACYSLLVTVAFIIFWV | 9 |
| transmembrane | | |
| domain | | |
| COSTIMULATORY DOMAINS | | |
| human 4-1BB | KRGRKKLLYIFKQPFMRPVQTTQEEDGCS | 6 |
| costimulatory | CRFPEEEEGGCEL | |
| domain | | |
| human CD28 | RSKRSRLLHSDYMNMTPRRPGPTRKHYQP | 10 |
| costimulatory | YAPPRDFAAYRS | |
| domain | | |
| ACTIVATION DOMAINS | | |
| human CD3zeta | RVKFSRSADAPAYQQGQNQLYNELNLGRREEYDVLDK | 7 |
| intracellular | RRGRDPEMGGKPRRKNPQEGLYNELQKDKMAEAYSEI | |
| signaling domain | GMKGERRRGKGHDGLYQGLSTATKDTYDALHMQALPP | |
| | R | |

### Nucleic Acids of the Present Disclosure

Nucleic acids encoding any of the CARs described herein are also provided. Nucleic acids encoding the CAR may be humanized. In some embodiments, the nucleic acid encoding a CAR provided herein is codon-optimized for expression in human cells. In some embodiments, the disclosure provides a full-length CAR cDNA or coding region. Included in the scope of the invention are nucleic acid sequences that encode functional portions of the CAR described herein. Functional portions encompass, for example, those parts of a CAR that retain the ability to recognize target cells, or detect, treat, or prevent a disease, to a similar extent, the same extent, or to a higher extent, as the parent CAR.

### Exemplary Anti-CD64 CAR Constructs

Disclosed herein are CARs that specifically bind to CD64. In some embodiments, the CAR comprises an antigen recognition domain that specifically binds human CD64, a hinge domain comprising or consisting of a CD8α hinge domain or a CD28 hinge domain, a transmembrane domain comprising or consisting of a CD8α transmembrane domain or a CD28 transmembrane domain; a costimulatory domain comprising or consisting of a 4-1BB costimulatory domain or a CD28 costimulatory domain; and an intracellular signaling domain comprising or consisting of a CD3zeta activation domain. Also disclosed herein are nucleic acid sequences encoding said CARs. In some embodiments, a T cell or population of T cells described herein is genetically modified to express at least one of the exemplary anti-CD64 CAR constructs described herein.

611 scfv CAR Constructs

An exemplary anti-CD64 CAR, “611-41BBz”, amino acid sequence is shown below. (CD8α signal peptide, CD64 scFv (611), CD8α hinge, CD8α transmembrane domain, 4-1BB signaling domain, CD3z signaling domain)

(SEQ ID NO: 11)
MALPVTALLLPLALLLHAARPQVQLVEAGGGVVQPGRSLRLSCAA
SGFIFSGYGMHWVRQAPGKGLEWVTVIWYDGSNKYYADSVKGRFT
ISRDNSKNTLYLQMNSLRAEDTAVYYCARDTGDRFFDYWGQGTLV
TVSSGGGGSGGGGSGGGGSEIVLTQSPATLSLSPGERATLSCRAS
QSVSSYLAWYQQKPGQAPRLLIYDASSRATGIPARFGGSGSGTDF
TLTISSLEPEDFAVYYCQLRSNWPPYTFGQGTKLEIKTSTTTPAP
RPPTPAPTIASQPLSLRPEACRPAAGGAVHTRGLDFACDIYIWAP
LAGTCGVLLLSLVITLYCKRGRKKLLYIFKQPFMRPVQTTQEEDG
CSCRFPEEEEGGCELSRVKFSRSADAPAYQQGQNQLYNELNLGRR
EEYDVLDKRRGRDPEMGGKPRRKNPQEGLYNELQKDKMAEAYSEI
GMKGERRRGKGHDGLYQGLSTATKDTYDALHMQALPPR*

In some embodiments, the anti-CD64 CAR provided herein may comprise, consist essentially of, or consist of an amino acid sequence having at least 90%, 91%, 92%, 93%, 94%, 95%, 96%, 97%, 98%, 99%, or 100% identity with the amino acid sequence of SEQ ID NO: 11.

An exemplary anti-CD64 CAR, “611-41BBz”, polynucleotide sequence is shown below. (CD8α signal peptide, CD64 scFv (611), CD8α hinge, CD8α transmembrane domain, 4-1BB signaling domain, CD3z signaling domain)

(SEQ ID NO: 17)
ATGGCTTTGCCTGTTACAGCTCTGCTGCTGCCCCTGGCTCTGCTT
CTGCATGCTGCTAGACCTCAGGTGCAGCTGGTTGAAGCTGGTGGC
GGAGTTGTGCAGCCTGGCAGATCTCTGAGACTGAGCTGTGCCGCC
AGCGGCTTCATCTTTAGCGGCTATGGCATGCACTGGGTCCGACAG
GCACCTGGCAAAGGCCTGGAATGGGTCACCGTGATTTGGTACGAC
GGCAGCAACAAGTACTACGCCGACAGCGTGAAGGGCAGATTCACC
ATCAGCCGGGACAACAGCAAGAACACCCTGTACCTGCAGATGAAC
AGCCTGAGAGCCGAGGACACCGCCGTGTACTACTGTGCCAGAGAC
ACCGGCGACAGATTCTTCGACTATTGGGGCCAGGGCACCCTCGTG
ACAGTTTCTTCTGGCGGCGGAGGATCTGGCGGAGGTGGAAGCGGA
GGCGGTGGATCTGAAATCGTGCTGACACAGAGCCCCGCCACACTG
TCACTTTCTCCAGGCGAAAGAGCCACACTGAGCTGCAGAGCCTCT
CAGAGCGTGTCCTCTTACCTGGCCTGGTATCAGCAGAAGCCTGGA
CAGGCTCCCCGGCTGCTGATCTACGATGCTTCTAGCAGAGCTACA
GGCATCCCCGCCAGATTTGGCGGCTCTGGCTCTGGCACTGATTTC
ACCCTGACCATAAGCAGCCTGGAACCTGAGGACTTTGCCGTGTAT
TATTGCCAGCTGCGGAGCAACTGGCCTCCTTACACATTTGGCCAG
GGGACCAAGCTGGAAATCAAGACTAGTACCACCACACCAGCTCCT
CGGCCACCTACTCCAGCTCCAACAATTGCCAGCCAGCCTCTGTCT
CTGAGGCCCGAAGCTTGTAGACCTGCTGCTGGCGGAGCCGTGCAT
ACAAGAGGACTGGATTTCGCCTGCGACATCTACATCTGGGCCCCT
CTGGCTGGAACATGTGGCGTGCTGCTGCTCAGCCTGGTCATCACC
CTGTACTGCAAGCGGGGCAGAAAGAAGCTGCTGTACATCTTCAAG
CAGCCCTTCATGCGGCCCGTGCAGACCACACAAGAGGAAGATGGC
TGCTCCTGCAGATTCCCCGAGGAAGAAGAAGGCGGCTGCGAGCTG
TCTAGAGTGAAGTTCAGCAGAAGCGCCGACGCTCCTGCCTATCAG
CAGGGACAGAATCAGCTGTACAACGAGCTGAACCTGGGGCGCAGA
GAAGAGTACGACGTGCTGGACAAGAGAAGAGGCAGGGACCCTGAG
ATGGGCGGCAAGCCCAGAAGAAAGAACCCTCAAGAGGGCCTGTAT
AATGAGCTGCAGAAAGACAAGATGGCCGAGGCCTACAGCGAGATC
GGAATGAAGGGCGAACGCAGAAGAGGAAAGGGCCACGACGGACTG
TATCAGGGCCTGAGCACAGCCACCAAGGACACCTATGATGCCCTG
CACATGCAGGCACTGCCTCCAAGATGA

In some embodiments, the anti-CD64 CAR provided herein is encoded by a polynucleotide sequence comprising, consisting essentially of, or consisting of a nucleic acid sequence having at least 90%, 91%, 92%, 93%, 94%, 95%, 96%, 97%, 98%, 99%, or 100% identity with the nucleic acid sequence of SEQ ID NO: 17.

An exemplary anti-CD64 CAR, “611-CD28z”, amino acid sequence is shown below. (CD8α signal peptide, CD64 scFv (611), CD28 hinge, CD28 transmembrane domain, CD28 signaling domain, CD3z signaling domain)

(SEQ ID NO: 14)
MALPVTALLLPLALLLHAARPQVQLVEAGGGVVQPGRSLRLSCAA
SGFIFSGYGMHWVRQAPGKGLEWVTVIWYDGSNKYYADSVKGRFT
ISRDNSKNTLYLQMNSLRAEDTAVYYCARDTGDRFFDYWGQGTLV
TVSSGGGGSGGGGSGGGGSEIVLTQSPATLSLSPGERATLSCRAS
QSVSSYLAWYQQKPGQAPRLLIYDASSRATGIPARFGGSGSGTDF
TLTISSLEPEDFAVYYCQLRSNWPPYTFGQGTKLEIKTSGAAAIE
VMYPPPYLDNEKSNGTIIHVKGKHLCPSPLFPGPSKPFWVLVVVG
GVLACYSLLVTVAFIIFWVRSKRSRLLHSDYMNMTPRRPGPTRKH
YQPYAPPRDFAAYRSRVKFSRSADAPAYQQGQNQLYNELNLGRRE
EYDVLDKRRGRDPEMGGKPRRKNPQEGLYNELQKDKMAEAYSEIG
MKGERRRGKGHDGLYQGLSTATKDTYDALHMQALPPR*

In some embodiments, the anti-CD64 CAR provided herein may comprise, consist essentially of, or consist of an amino acid sequence having at least 90%, 91%, 92%, 93%, 94%, 95%, 96%, 97%, 98%, 99%, or 100% identity with the amino acid sequence of SEQ ID NO: 14.

An exemplary anti-CD64 CAR, “611-CD28z”, polynucleotide sequence is shown below. (CD8α signal peptide. CD64 scFv (611), CD28 hinge, CD28 transmembrane domain. CD28 signaling domain, CD3z signaling domain)

(SEQ ID NO: 18)
ATGGCTTTGCCTGTTACAGCTCTGCTGCTGCCCCTGGCTCTGCTT
CTGCATGCTGCTAGACCTCAGGTGCAGCTGGTTGAAGCTGGTGGC
GGAGTTGTGCAGCCTGGCAGATCTCTGAGACTGAGCTGTGCCGCC
AGCGGCTTCATCTTTAGCGGCTATGGCATGCACTGGGTCCGACAG
GCACCTGGCAAAGGCCTGGAATGGGTCACCGTGATTTGGTACGAC
GGCAGCAACAAGTACTACGCCGACAGCGTGAAGGGCAGATTCACC
ATCAGCCGGGACAACAGCAAGAACACCCTGTACCTGCAGATGAAC
AGCCTGAGAGCCGAGGACACCGCCGTGTACTACTGTGCCAGAGAC
ACCGGCGACAGATTCTTCGACTATTGGGGCCAGGGCACCCTCGTG
ACAGTTTCTTCTGGCGGCGGAGGATCTGGCGGAGGTGGAAGCGGA
GGCGGTGGATCTGAAATCGTGCTGACACAGAGCCCCGCCACACTG
TCACTTTCTCCAGGCGAAAGAGCCACACTGAGCTGCAGAGCCTCT
CAGAGCGTGTCCTCTTACCTGGCCTGGTATCAGCAGAAGCCTGGA
CAGGCTCCCCGGCTGCTGATCTACGATGCTTCTAGCAGAGCTACA
GGCATCCCCGCCAGATTTGGCGGCTCTGGCTCTGGCACTGATTTC
ACCCTGACCATAAGCAGCCTGGAACCTGAGGACTTTGCCGTGTAT
TATTGCCAGCTGCGGAGCAACTGGCCTCCTTACACATTTGGCCAG
GGGACCAAGCTGGAAATCAAGACTAGTGGCGCCGCTGCTATTGAA
GTGATGTACCCTCCTCCTTACCTGGACAACGAGAAGTCCAACGGC
ACCATCATCCACGTGAAGGGCAAGCACCTGTGTCCTTCTCCACTG
TTCCCCGGACCTAGCAAGCCTTTCTGGGTGCTCGTTGTTGTTGGC
GGCGTGCTGGCCTGTTATTCCCTGCTGGTTACCGTGGCCTTCATC
ATCTTTTGGGTCCGAAGCAAGCGGAGCCGGCTGCTGCACAGCGAC
TACATGAACATGACCCCTAGACGGCCCGGACCAACCAGAAAGCAC
TACCAGCCTTACGCTCCTCCTAGAGACTTCGCCGCCTACCGGTCT
AGAGTGAAGTTCAGCAGATCCGCCGACGCTCCTGCCTATCAGCAG
GGACAGAACCAGCTGTACAACGAGCTGAACCTGGGGAGAAGAGAA
GAGTACGACGTGCTGGACAAGCGGAGAGGCAGAGATCCTGAGATG
GGCGGCAAGCCCAGACGGAAGAATCCTCAAGAGGGCCTGTATAAT
GAGCTGCAGAAAGACAAGATGGCCGAGGCCTACAGCGAGATCGGA
ATGAAGGGCGAGCGCAGAAGAGGCAAGGGACACGATGGACTGTAC
CAGGGCCTGAGCACCGCCACCAAGGATACCTATGATGCCCTGCAC
ATGCAGGCCCTGCCTCCAAGATGA

In some embodiments, the anti-CD64 CAR provided herein is encoded by a polynucleotide sequence comprising, consisting essentially of. or consisting of a nucleic acid sequence having at least 90%, 91%, 92%, 93%, 94%, 95%, 96%, 97%, 98%, 99%, or 100% identity with the nucleic acid sequence of SEQ ID NO: 18.

H22 scfv CAR Constructs

An exemplary anti-CD64 CAR, “H22-41BBz”, amino acid sequence is shown below. (CD8α signal peptide. CD64 scFv (H22), CD8α hinge, CD8α transmembrane domain. 4-1BB signaling domain, CD3z signaling domain)

(SEQ ID NO: 12)
MALPVTALLLPLALLLHAARPQVQLVESGGGWQPGRSLRLSCSSS
GFIFSDNYMYWVRQAPGKGLEWVATISDGGSYTYYPDSVKGRFTI
SRDNSKNTLFLQMDSLRPEDTGVYFCARGYYRYEGAMDYWGQGTP
VTVSSGGGGSGGGGSGGGGSDIQLTQSPSSLSASVGDRVTITCKS
SQSVLYSSNQKNYLAWYQQKPGKAPKLLIYWASTRESGVPSRFSG
SGSGTDFTFTISSLQPEDIATYYCHQYLSSWTFGQGTKLEIKTST
TTPAPRPPTPAPTIASQPLSLRPEACRPAAGGAVHTRGLDFACDI
YIWAPLAGTCGVLLLSLVITLYCKRGRKKLLYIFKQPFMRPVQTT
QEEDGCSCRFPEEEEGGCELSRVKFSRSADAPAYQQGQNQLYNEL
NLGRREEYDVLDKRRGRDPEMGGKPRRKNPQEGLYNELQKDKMAE
AYSEIGMKGERRRGKGHDGLYQGLSTATKDTYDALHMQALPPR*

In some embodiments, the anti-CD64 CAR provided herein may comprise, consist essentially of, or consist of an amino acid sequence having at least 90%, 91%, 92%, 93%, 94%, 95%, 96%, 97%, 98%, 99%, or 100% identity with the amino acid sequence of SEQ ID NO: 12.

An exemplary anti-CD64 CAR, “H22-41BBz”, polynucleotide sequence is shown below. (CD8α signal peptide, CD64 scFv (H22), CD8α hinge, CD8α transmembrane domain, 4-1BB signaling domain, CD3z signaling domain)

(SEQ ID NO: 19)
ATGGCTTTGCCTGTTACAGCTCTGCTGCTGCCCCTGGCTCTGCTT
CTGCATGCTGCTAGACCTCAGGTGCAGCTGGTGGAATCTGGCGGA
GGATGGCAGCCTGGCAGAAGCCTGAGACTGAGCTGTAGCAGCAGC
GGCTTCATCTTCAGCGACAACTACATGTACTGGGTCCGACAGGCC
CCTGGCAAAGGCCTTGAATGGGTCGCCACAATCTCTGACGGCGGC
AGCTACACCTACTATCCCGACTCTGTGAAGGGCAGATTCACCATC
AGCCGGGACAACAGCAAGAATACCCTGTTCCTGCAGATGGACAGC
CTGCGGCCTGAAGATACCGGCGTGTACTTTTGCGCCAGAGGCTAC
TACAGATACGAGGGCGCCATGGACTATTGGGGCCAGGGAACACCT
GTGACCGTTTCTAGCGGCGGAGGTGGAAGCGGAGGCGGAGGTAGT
GGTGGTGGCGGATCTGACATTCAGCTGACACAGAGCCCTAGCAGC
CTGTCTGCCTCTGTGGGCGACAGAGTGACCATCACATGCAAGAGC
AGCCAGAGCGTGCTGTACTCCAGCAACCAGAAGAACTACCTGGCC
TGGTATCAGCAGAAGCCCGGCAAGGCTCCTAAGCTGCTGATCTAC
TGGGCCAGCACCAGAGAAAGCGGCGTGCCAAGCAGATTTTCTGGC
AGCGGCTCTGGCACCGACTTCACCTTCACCATAAGCTCCCTGCAG
CCTGAGGACATTGCCACCTACTACTGCCACCAGTACCTGAGCAGC
TGGACCTTTGGCCAGGGCACCAAGCTGGAAATCAAGACTAGTACC
ACCACACCAGCTCCTCGGCCACCTACTCCAGCTCCAACAATTGCC
AGCCAGCCTCTGTCTCTGAGGCCCGAAGCTTGTAGACCTGCTGCT
GGCGGAGCCGTGCATACAAGAGGACTGGATTTCGCCTGCGACATC
TACATCTGGGCCCCTCTGGCTGGAACATGTGGCGTGCTGCTGCTC
AGCCTGGTCATCACCCTGTACTGCAAGCGGGGCAGAAAGAAGCTG
CTGTACATCTTCAAGCAGCCCTTCATGCGGCCCGTGCAGACCACA
CAAGAGGAAGATGGCTGCTCCTGCAGATTCCCCGAGGAAGAAGAA
GGCGGCTGCGAGCTGTCTAGAGTGAAGTTCAGCAGAAGCGCCGAC
GCTCCTGCCTATCAGCAGGGACAGAATCAGCTGTACAACGAGCTG
AACCTGGGGCGCAGAGAAGAGTACGACGTGCTGGACAAGAGAAGA
GGCAGGGACCCTGAGATGGGCGGCAAGCCCAGAAGAAAGAACCCT
CAAGAGGGCCTGTATAATGAGCTGCAGAAAGACAAGATGGCCGAG
GCCTACAGCGAGATCGGAATGAAGGGCGAACGCAGAAGAGGAAAG
GGCCACGACGGACTGTATCAGGGCCTGAGCACAGCCACCAAGGAC
ACCTATGATGCCCTGCACATGCAGGCACTGCCTCCAAGATGA

In some embodiments, the anti-CD64 CAR provided herein is encoded by a polynucleotide sequence comprising, consisting essentially of, or consisting of a nucleic acid sequence having at least 90%, 91%, 92%, 93%, 94%, 95%, 96%, 97%, 98%, 99%, or 100% identity with the nucleic acid sequence of SEQ ID NO: 19.

An exemplary anti-CD64 CAR, “H22-CD28z”, amino acid sequence is shown below. (CD8α signal peptide. CD64 scFv (H22), CD28 hinge, CD28 transmembrane domain. CD28 signaling domain, CD3z signaling domain)

(SEQ ID NO: 15)
MALPVTALLLPLALLLHAARPQVQLVESGGGWQPGRSLRLSCSSS
GFIFSDNYMYWVRQAPGKGLEWVATISDGGSYTYYPDSVKGRFTI
SRDNSKNTLFLQMDSLRPEDTGVYFCARGYYRYEGAMDYWGQGTP
VTVSSGGGGSGGGGSGGGGSDIQLTQSPSSLSASVGDRVTITCKS
SQSVLYSSNQKNYLAWYQQKPGKAPKLLIYWASTRESGVPSRFSG
SGSGTDFTFTISSLQPEDIATYYCHQYLSSWTFGQGTKLEIKTSG
AAAIEVMYPPPYLDNEKSNGTIIHVKGKHLCPSPLFPGPSKPFWV
LVVVGGVLACYSLLVTVAFIIFWVRSKRSRLLHSDYMNMTPRRPG
PTRKHYQPYAPPRDFAAYRSRVKFSRSADAPAYQQGQNQLYNELN
LGRREEYDVLDKRRGRDPEMGGKPRRKNPQEGLYNELQKDKMAEA
YSEIGMKGERRRGKGHDGLYQGLSTATKDTYDALHMQALPPR*

In some embodiments, the anti-CD64 CAR provided herein may comprise, consist essentially of, or consist of an amino acid sequence having at least 90%, 91%, 92%, 93%, 94%, 95%, 96%, 97%, 98%, 99%, or 100% identity with the amino acid sequence of SEQ ID NO: 15.

An exemplary anti-CD64 CAR, “H22-CD28z”, polynucleotide sequence is shown below. (CD8α signal peptide, CD64 scFv (H22), CD28 hinge, CD28 transmembrane domain, CD28 signaling domain, CD3z signaling domain)

(SEQ ID NO: 20)
ATGGCTTTGCCTGTTACAGCTCTGCTGCTGCCCCTGGCTCTGCTT
CTGCATGCTGCTAGACCTCAGGTGCAGCTGGTGGAATCTGGCGGA
GGATGGCAGCCTGGCAGAAGCCTGAGACTGAGCTGTAGCAGCAGC
GGCTTCATCTTCAGCGACAACTACATGTACTGGGTCCGACAGGCC
CCTGGCAAAGGCCTTGAATGGGTCGCCACAATCTCTGACGGCGGC
AGCTACACCTACTATCCCGACTCTGTGAAGGGCAGATTCACCATC
AGCCGGGACAACAGCAAGAATACCCTGTTCCTGCAGATGGACAGC
CTGCGGCCTGAAGATACCGGCGTGTACTTTTGCGCCAGAGGCTAC
TACAGATACGAGGGCGCCATGGACTATTGGGGCCAGGGAACACCT
GTGACCGTTTCTAGCGGCGGAGGTGGAAGCGGAGGCGGAGGTAGT
GGTGGTGGCGGATCTGACATTCAGCTGACACAGAGCCCTAGCAGC
CTGTCTGCCTCTGTGGGCGACAGAGTGACCATCACATGCAAGAGC
AGCCAGAGCGTGCTGTACTCCAGCAACCAGAAGAACTACCTGGCC
TGGTATCAGCAGAAGCCCGGCAAGGCTCCTAAGCTGCTGATCTAC
TGGGCCAGCACCAGAGAAAGCGGCGTGCCAAGCAGATTTTCTGGC
AGCGGCTCTGGCACCGACTTCACCTTCACCATAAGCTCCCTGCAG
CCTGAGGACATTGCCACCTACTACTGCCACCAGTACCTGAGCAGC
TGGACCTTTGGCCAGGGCACCAAGCTGGAAATCAAGACTAGTGGC
GCCGCTGCTATTGAAGTGATGTACCCTCCTCCTTACCTGGACAAC
GAGAAGTCCAACGGCACCATCATCCACGTGAAGGGCAAGCACCTG
TGTCCTTCTCCACTGTTCCCCGGACCTAGCAAGCCTTTCTGGGTG
CTCGTTGTTGTTGGCGGCGTGCTGGCCTGTTATTCCCTGCTGGTT
ACCGTGGCCTTCATCATCTTTTGGGTCCGAAGCAAGCGGAGCCGG
CTGCTGCACAGCGACTACATGAACATGACCCCTAGACGGCCCGGA
CCAACCAGAAAGCACTACCAGCCTTACGCTCCTCCTAGAGACTTC
GCCGCCTACCGGTCTAGAGTGAAGTTCAGCAGATCCGCCGACGCT
CCTGCCTATCAGCAGGGACAGAACCAGCTGTACAACGAGCTGAAC
CTGGGGAGAAGAGAAGAGTACGACGTGCTGGACAAGCGGAGAGGC
AGAGATCCTGAGATGGGCGGCAAGCCCAGACGGAAGAATCCTCAA
GAGGGCCTGTATAATGAGCTGCAGAAAGACAAGATGGCCGAGGCC
TACAGCGAGATCGGAATGAAGGGCGAGCGCAGAAGAGGCAAGGGA
CACGATGGACTGTACCAGGGCCTGAGCACCGCCACCAAGGATACC
TATGATGCCCTGCACATGCAGGCCCTGCCTCCAAGATGA

In some embodiments, the anti-CD64 CAR provided herein is encoded by a polynucleotide sequence comprising, consisting essentially of, or consisting of a nucleic acid sequence having at least 90%, 91%, 92%, 93%, 94%, 95%, 96%, 97%, 98%, 99%, or 100% identity with the nucleic acid sequence of SEQ ID NO: 20.

M22 scfv CAR Constructs

An exemplary anti-CD64 CAR, “M22-41BBz”, amino acid sequence is shown below. (CD8α signal peptide, CD64 scFv (M22), CD8α hinge, CD8α transmembrane domain, 4-1BB signaling domain, CD3z signaling domain)

(SEQ ID NO: 13)
MALPVTALLLPLALLLHAARPEVQLVESGGGLVKPGGSLRLSCVA
SGFIFSDNYMYWVRQTPEKRLEWVATISDGGSYTYYPDSVKGRFT
ISRDNAKNNLYLQMSSLKSEDTAIYYCARGYYRYEGAMDYWGQGT
SVTVSSGGGGSGGGGSGGGGSNIVMTQSPSSLAVSAGEKVTMSCK
SSQSVLYSSNQKNYLAWYQQKPGQSPKLLIYWASTRESGVPDRFT
GSGSGTDFTLTISSVQAEDLAVYYCHQYLSSWTFGGGTKLEIKTS
TTTPAPRPPTPAPTIASQPLSLRPEACRPAAGGAVHTRGLDFACD
IYIWAPLAGTCGVLLLSLVITLYCKRGRKKLLYIFKQPFMRPVQT
TQEEDGCSCRFPEEEEGGCELSRVKFSRSADAPAYQQGQNQLYNE
LNLGRREEYDVLDKRRGRDPEMGGKPRRKNPQEGLYNELQKDKMA
EAYSEIGMKGERRRGKGHDGLYQGLSTATKDTYDALHMQALPPR*

In some embodiments, the anti-CD64 CAR provided herein may comprise, consist essentially of, or consist of an amino acid sequence having at least 90%, 91%, 92%, 93%, 94%, 95%, 96%, 97%, 98%, 99%, or 100% identity with the amino acid sequence of SEQ ID NO: 13.

An exemplary anti-CD64 CAR, “M22-41BBz”, polynucleotide sequence is shown below. (CD8α signal peptide, CD64 scFv (M22), CD8α hinge, CD8α transmembrane domain, 4-1BB signaling domain, CD3z signaling domain)

(SEQ ID NO: 21)
ATGGCTCTGCCTGTTACAGCTCTGCTGCTGCCTCTGGCTCTGCTT
CTGCATGCCGCTAGACCTGAGGTGCAGCTGGTTGAATCTGGCGGC
GGACTTGTGAAGCCTGGCGGATCTCTGAGACTGAGCTGTGTGGCC
AGCGGCTTCATCTTCAGCGACAACTACATGTACTGGGTCCGACAG
ACCCCTGAGAAGCGGCTGGAATGGGTCGCCACAATCTCTGATGGC
GGCAGCTACACCTACTATCCCGACAGCGTGAAGGGCAGATTCACC
ATCAGCCGGGACAACGCCAAGAACAACCTGTACCTGCAGATGAGC
AGCCTGAAGTCCGAGGACACCGCCATCTACTACTGCGCCAGAGGC
TACTACAGATACGAGGGCGCCATGGACTATTGGGGCCAGGGAACA
AGCGTGACAGTGTCTAGCGGAGGCGGAGGATCAGGTGGCGGTGGA
TCTGGCGGTGGCGGCTCTAATATCGTGATGACACAGAGCCCTAGC
AGCCTGGCTGTGTCTGCCGGCGAGAAAGTGACCATGAGCTGCAAG
AGCAGCCAGAGCGTGCTGTACTCCAGCAACCAGAAGAACTACCTG
GCCTGGTATCAGCAGAAGCCCGGACAGTCTCCCAAGCTGCTGATC
TACTGGGCCAGCACCAGAGAAAGCGGCGTGCCCGATAGATTCACA
GGCAGCGGCTCTGGCACCGACTTCACCCTGACAATCAGCTCTGTG
CAGGCCGAGGATCTGGCCGTGTACTACTGTCACCAGTACCTGAGC
AGCTGGACCTTTGGCGGAGGCACCAAGCTGGAAATCAAGACTAGT
ACCACCACACCAGCTCCTCGGCCACCTACTCCAGCTCCAACAATT
GCCAGCCAGCCTCTGTCTCTGAGGCCCGAAGCTTGTAGACCTGCT
GCTGGCGGAGCCGTGCATACAAGAGGACTGGATTTCGCCTGCGAC
ATCTACATCTGGGCCCCTCTGGCTGGAACATGTGGCGTGCTGCTG
CTCAGCCTGGTCATCACCCTGTACTGCAAGCGGGGCAGAAAGAAG
CTGCTGTACATCTTCAAGCAGCCCTTCATGCGGCCCGTGCAGACC
ACACAAGAGGAAGATGGCTGCTCCTGCAGATTCCCCGAGGAAGAA
GAAGGCGGCTGCGAGCTGTCTAGAGTGAAGTTCAGCAGAAGCGCC
GACGCTCCTGCCTATCAGCAGGGACAGAATCAGCTGTACAACGAG
CTGAACCTGGGGCGCAGAGAAGAGTACGACGTGCTGGACAAGAGA
AGAGGCAGGGACCCTGAGATGGGCGGCAAGCCCAGAAGAAAGAAC
CCTCAAGAGGGCCTGTATAATGAGCTGCAGAAAGACAAGATGGCC
GAGGCCTACAGCGAGATCGGAATGAAGGGCGAACGCAGAAGAGGA
AAGGGCCACGACGGACTGTATCAGGGCCTGAGCACAGCCACCAAG
GACACCTATGATGCCCTGCACATGCAGGCACTGCCTCCAAGATGA

In some embodiments, the anti-CD64 CAR provided herein is encoded by a polynucleotide sequence comprising, consisting essentially of, or consisting of a nucleic acid sequence having at least 90%, 91%, 92%, 93%, 94%, 95%, 96%, 97%, 98%, 99%, or 100% identity with the nucleic acid sequence of SEQ ID NO: 21.

An exemplary anti-CD64 CAR, “M22-CD28z”, amino acid sequence is shown below. (CD8α signal peptide. CD64 scFv (M22), CD28 hinge, CD28 transmembrane domain. CD28 signaling domain, CD3z signaling domain)

(SEQ ID NO: 16)
MALPVTALLLPLALLLHAARPEVQLVESGGGLVKPGGSLRLSCVA
SGFIFSDNYMYWVRQTPEKRLEWVATISDGGSYTYYPDSVKGRFT
ISRDNAKNNLYLQMSSLKSEDTAIYYCARGYYRYEGAMDYWGQGT
SVTVSSGGGGSGGGGSGGGGSNIVMTQSPSSLAVSAGEKVTMSCK
SSQSVLYSSNQKNYLAWYQQKPGQSPKLLIYWASTRESGVPDRFT
GSGSGTDFTLTISSVQAEDLAVYYCHQYLSSWTFGGGTKLEIKTS
GAAAIEVMYPPPYLDNEKSNGTIIHVKGKHLCPSPLFPGPSKPFW
VLVVVGGVLACYSLLVTVAFIIFWVRSKRSRLLHSDYMNMTPRRP
GPTRKHYQPYAPPRDFAAYRSRVKFSRSADAPAYQQGQNQLYNEL
NLGRREEYDVLDKRRGRDPEMGGKPRRKNPQEGLYNELQKDKMAE
AYSEIGMKGERRRGKGHDGLYQGLSTATKDTYDALHMQALPPR*

In some embodiments, the anti-CD64 CAR provided herein may comprise, consist essentially of, or consist of an amino acid sequence having at least 90%, 91%, 92%, 93%, 94%, 95%, 96%, 97%, 98%, 99%, or 100% identity with the amino acid sequence of SEQ ID NO: 16.

An exemplary anti-CD64 CAR, “M22-CD28z”, polynucleotide sequence is shown below. (CD8α signal peptide, CD64 scFv (M22), CD28 hinge, CD28 transmembrane domain, CD28 signaling domain, CD3z signaling domain)

(SEQ ID NO: 22)
ATGGCTCTGCCTGTTACAGCTCTGCTGCTGCCTCTGGCTCTGCTT
CTGCATGCCGCTAGACCTGAGGTGCAGCTGGTTGAATCTGGCGGC
GGACTTGTGAAGCCTGGCGGATCTCTGAGACTGAGCTGTGTGGCC
AGCGGCTTCATCTTCAGCGACAACTACATGTACTGGGTCCGACAG
ACCCCTGAGAAGCGGCTGGAATGGGTCGCCACAATCTCTGATGGC
GGCAGCTACACCTACTATCCCGACAGCGTGAAGGGCAGATTCACC
ATCAGCCGGGACAACGCCAAGAACAACCTGTACCTGCAGATGAGC
AGCCTGAAGTCCGAGGACACCGCCATCTACTACTGCGCCAGAGGC
TACTACAGATACGAGGGCGCCATGGACTATTGGGGCCAGGGAACA
AGCGTGACAGTGTCTAGCGGAGGCGGAGGATCAGGTGGCGGTGGA
TCTGGCGGTGGCGGCTCTAATATCGTGATGACACAGAGCCCTAGC
AGCCTGGCTGTGTCTGCCGGCGAGAAAGTGACCATGAGCTGCAAG
AGCAGCCAGAGCGTGCTGTACTCCAGCAACCAGAAGAACTACCTG
GCCTGGTATCAGCAGAAGCCCGGACAGTCTCCCAAGCTGCTGATC
TACTGGGCCAGCACCAGAGAAAGCGGCGTGCCCGATAGATTCACA
GGCAGCGGCTCTGGCACCGACTTCACCCTGACAATCAGCTCTGTG
CAGGCCGAGGATCTGGCCGTGTACTACTGTCACCAGTACCTGAGC
AGCTGGACCTTTGGCGGAGGCACCAAGCTGGAAATCAAGACTAGT
GGCGCCGCTGCTATTGAAGTGATGTACCCTCCTCCTTACCTGGAC
AACGAGAAGTCCAACGGCACCATCATCCACGTGAAGGGCAAGCAC
CTGTGTCCTTCTCCACTGTTCCCCGGACCTAGCAAGCCTTTCTGG
GTGCTCGTTGTTGTTGGCGGCGTGCTGGCCTGTTATTCCCTGCTG
GTTACCGTGGCCTTCATCATCTTTTGGGTCCGAAGCAAGCGGAGC
CGGCTGCTGCACAGCGACTACATGAACATGACCCCTAGACGGCCC
GGACCAACCAGAAAGCACTACCAGCCTTACGCTCCTCCTAGAGAC
TTCGCCGCCTACCGGTCTAGAGTGAAGTTCAGCAGATCCGCCGAC
GCTCCTGCCTATCAGCAGGGACAGAACCAGCTGTACAACGAGCTG
AACCTGGGGAGAAGAGAAGAGTACGACGTGCTGGACAAGCGGAGA
GGCAGAGATCCTGAGATGGGCGGCAAGCCCAGACGGAAGAATCCT
CAAGAGGGCCTGTATAATGAGCTGCAGAAAGACAAGATGGCCGAG
GCCTACAGCGAGATCGGAATGAAGGGCGAGCGCAGAAGAGGCAAG
GGACACGATGGACTGTACCAGGGCCTGAGCACCGCCACCAAGGAT
ACCTATGATGCCCTGCACATGCAGGCCCTGCCTCCAAGATGA

In some embodiments, the anti-CD64 CAR provided herein is encoded by a polynucleotide sequence comprising or consisting of a nucleic acid sequence having at least 90%, 91%, 92%, 93%, 94%, 95%, 96%, 97%, 98%, 99%, or 100% identity with the nucleic acid sequence of SEQ ID NO: 22.

In some embodiments, the genetically engineered cells include additional CARs, including activating or stimulatory CARs, co-stimulatory CARs (see, e.g., PCT Publ. No. WO 2014/055668), and/or inhibitory CARs (iCARs, see, e.g., Fedorov et al., 2013). The CARs generally include an extracellular antigen (or ligand) recognition domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone. For example, once an antigen is recognized by the extracellular antigen recognition domain, the intracellular signaling components transmit an activation signal to the T cell that induces the T cell to destroy a targeted tumor cell.

In embodiments, the CARs described herein contain additional amino acids at the amino or carboxy terminus of the portion, or at both termini. which additional amino acids are not found in the amino acid sequence of the parent CAR. Desirably, the additional amino acids do not interfere with the biological function of the functional portion, e.g., recognize target cells, detect cancer, treat or prevent cancer, etc. More desirably, the additional amino acids enhance the biological activity of the CAR, as compared to the biological activity of the parent CAR.

The term “functional variant,” as used herein in reference to a CAR, refers to a CAR, a polypeptide, or a protein having substantial or significant sequence identity or similarity to the CAR encoded by a nucleic acid sequence, which functional variant retains the biological activity of the CAR of which it is a variant. Functional variants encompass, for example, those variants of the CAR described herein (the parent CAR) that retain the ability to recognize target cells to a similar extent, the same extent, or to a higher extent, as the parent CAR. In reference to a nucleic acid sequence encoding the parent CAR, a nucleic acid sequence encoding a functional variant of the CAR can be for example, about 10% identical, about 25% identical, about 30% identical, about 50% identical, about 65% identical, about 80% identical, about 90% identical, about 95% identical, or about 99% identical to the nucleic acid sequence encoding the parent CAR.

A CAR described herein include (including functional portions and functional variants thereof) glycosylated, amidated, carboxylated, phosphorylated, esterified, N-acylated, cyclized via, e.g., a disulfide bridge, or converted into an acid addition salt and/or optionally dimerized or polymerized.

As described herein, the amino acid sequences described herein can include amino acid modifications (e.g., the articulated number of amino acid modifications). Such amino acid modifications can include, without limitation, amino acid substitutions, amino acid deletions, amino acid additions, and combinations. In some cases, an amino acid modification can be made to improve the binding and/or contact with an antigen and/or to improve a functional activity of a CAR provided herein. In some cases, an amino acid substitution within an articulated sequence identifier can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution within an articulated sequence identifier can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

Methods for generating an amino acid sequence variant (e.g., an amino acid sequence that includes one or more modifications with respect to an articulated sequence identifier) can include site-specific mutagenesis or random mutagenesis (e.g., by PCR) of a nucleic acid encoding the antibody or fragment thereof. See, for example, Zoller, Curr. Opin. Biotechnol. 3: 348-354 (1992). Both naturally occurring and non-naturally occurring amino acids (e.g., artificially-derivatized amino acids) can be used to generate an amino acid sequence variant provided herein.

### Immune Cells of the Present Disclosure

The present disclosure provides cells and populations of cells comprising the CAR constructs described above and/or the nucleic acid molecules encoding said CAR constructs.

Accordingly, the present disclosure provides populations of genetically modified immune cells (e.g. T cells) engineered to express a CAR described above and/or a polynucleotide encoding said CAR The immune cells may be T cells (e.g., regulatory T cells, CD4⁺ T cells, CD8⁺ T cells, or gamma-delta T cells), NK cells, invariant NK cells, NKT cells, stem cells (e.g., mesenchymal stem cells (MSCs) or induced pluripotent stem (iPSC) cells). In some embodiments, the cells are monocytes or granulocytes, e.g., myeloid cells, macrophages, neutrophils, dendritic cells, mast cells, eosinophils, and/or basophils. Also provided herein are methods of producing and engineering the immune cells and methods of using and administering the cells to a subject, in which case the cells may be autologous or allogeneic. Thus, the immune cells may be used as immunotherapy, such as to target cancer cells.

In some embodiments, the cells comprise one or more nucleic acids introduced via genetic engineering that encode one or more antigen receptors. and genetically engineered products of such nucleic acids. In some embodiments, the nucleic acids are heterologous. In some embodiments, the nucleic acids are not naturally occurring, such as a nucleic acid not found in nature (e.g., chimeric).

The immune cells may be isolated from subjects, particularly human subjects. The immune cells can be obtained from a subject of interest, such as a subject suspected of having a particular disease or condition, a subject suspected of having a predisposition to a particular disease or condition, or a subject who is undergoing therapy for a particular disease or condition. The immune cells may be enriched/purified from any tissue where they reside including, but not limited to, blood (including blood collected by blood banks or cord blood banks), spleen, bone marrow, tissues removed and/or exposed during surgical procedures, and tissues obtained via biopsy procedures. Tissues/organs from which the immune cells are enriched, isolated, and/or purified may be isolated from both living and non-living subjects, wherein the non-living subjects are organ donors. The isolated immune cells may be used directly, or they can be stored for a period of time, such as by freezing. In some embodiments, the immune cells are isolated from blood, such as peripheral blood or cord blood. In some embodiments, immune cells isolated from cord blood have enhanced immunomodulation capacity, such as measured by CD4-positive or CD8-positive T cell suppression. In specific aspects, the immune cells are isolated from pooled blood, particularly pooled cord blood, for enhanced immunomodulation capacity. The pooled blood may be from 2 or more sources, such as 3, 4, 5, 6, 7, 8, 9, 10 or more sources (e.g., donor subjects).

The population of immune cells can be obtained from a subject in need of therapy or suffering from a disease associated with reduced immune cell activity. Thus, the cells will be autologous to the subject in need of therapy. Alternatively, the population of immune cells can be obtained from a donor. The immune cell population can be harvested from the peripheral blood, cord blood, bone marrow, spleen, or any other organ/tissue in which immune cells reside in said subject or donor. The immune cells can be isolated from a pool of subjects and/or donors, such as from pooled cord blood. The population of immune cells can be derived from induced pluripotent stem cells (iPSCs) and/or any other stem cell known in the art. In some aspects, the iPSCS and/or stem cells used to derive the population of immune cells can be obtained from a subject in need of therapy or suffering from a disease associate with reduced immune cell activity, thus these IPSCs and/or stem cells will be autologous to the subject in need of therapy. Alternatively, the iPSCs and/or stem cells can be obtained from a donor and therefore be allogeneic to the subject in need of therapy.

When the population of immune cells is obtained from a donor distinct from the subject, the donor is preferably allogeneic, provided the cells obtained are subject-compatible in that they can be introduced into the subject. Allogeneic donor cells are may or may not be human leukocyte antigen (HLA)-compatible. To be rendered subject-compatible, allogeneic cells can be treated to reduce immunogenicity.

### T Cells

T cells play a major role in cell-mediated-immunity (no antibody involvement). Its T cell receptors (TCR) differentiate themselves from other lymphocyte types. The thymus, a specialized organ of the immune system, is primarily responsible for the T cell's maturation. There are six types of T cells, namely: Helper T cells (e.g CD4+ cells), Cytotoxic T cells (also known as TC, cytotoxic T lymphocyte, CTL, T killer cell, cytolytic T cell, CD8+ T cells or killer T cell), Memory T cells ((i) sten memory TSCM cells, like naive cells, are CD45RO−, CCR7+, CD45RA+, CD62L+(L-selectin), CD27+, CD28+ and IL-7Ra+, but they also express large amounts of CD95, IL-2R, CXCR3, and LFA-1, and show numerous functional attributes distinctive of memory cells); (ii) central memory TCM cells express L-selectin and the CCR7, they secrete IL-2. but not IFNg or IL-4, and (iii) effector memory TEM cells, however, do not express L-selectin or CCR7 but produce effector cytokines like IFNg and IL-4), Regulatory T cells (Tregs, suppressor T cells, or CD4+CD25+ regulatory T cells), Natural Killer T cells (NKT) and Gamma Delta T cells.

The T cells of the immunotherapy can come from any source known in the art. For example. T cells can be differentiated in vitro from a hematopoictic stem cell population. or T cells can be obtained from a subject. T cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748. which is herein incorporated by references in its entirety.

### CAR Expression Levels

The present disclosure provides a population of engineered T cells, wherein a plurality of the engineered T cells of the population comprise any chimeric stimulatory receptor (CAR) disclosed herein. The present disclosure also provides a composition comprising a population of T cells, wherein a plurality of the T cells of the population comprises a non-naturally occurring CAR comprising, consisting essentially of, or consisting of a chimeric antigen receptor (CAR) comprising an antigen recognition domain that binds to CD64, a transmembrane domain, and an intracellular signaling domain. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprise any chimeric stimulatory receptor (CAR) disclosed herein. In some embodiments, the CAR polypeptide is expressed at a copy number of at least 1, 2, 3, 4, 5, 6, 7, 8, 9, 10, 20, 30, 40, 50, 60, 70, 80, 90, or 100 copies per cell. In some embodiments, the nucleic acid encoding the CAR is integrated into the genome at a copy number of at least 1, 2, 3, 4, 5, 6, 7, 8, 9, 10, 20, or 30 copies per cell.

In some embodiments, a population of genetically engineered T cells as disclosed herein exhibits T cell functions (e.g., effector functions). In some embodiments, the population is cytotoxic to CD64-expressing cells (e.g., CD64-positive tumor cells, CD64-low tumor cells). Effector function of a T cell, for example, may be cytolytic activity or helper activity including the secretion of cytokines. In some embodiments, the population exhibits one or more T cell effector functions at a level that is least 3-4-fold higher than the functions exhibited by a population of T cells not expressing the CAR.

### Methods of Making the Cells of the Present Disclosure

Chimeric antigen receptors may be readily inserted into and expressed by immune cells, (e.g., T cells). In certain embodiments, cells (e.g., immune cells such as T cells) are obtained from a donor subject. In some embodiments, the donor subject is a human patient afflicted with a cancer or a tumor. In other embodiments, the donor subject is a human patient not afflicted with a cancer or a tumor. In some embodiments, an engineered cell is autologous to a subject. In some embodiments, an engineered cell is allogeneic to a subject.

The cell of the present disclosure may be obtained through any source known in the art. For example, T cells can be differentiated in vitro from a hematopoietic stem cell population, or T cells can be obtained from a subject. T cells can be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. In certain embodiments, the cells collected by apheresis are washed to remove the plasma fraction and placed in an appropriate buffer or media for subsequent processing. In some embodiments, the cells are washed with PBS. As will be appreciated, a washing step can be used, such as by using a semiautomated flowthrough centrifuge, e.g., the Cobe™ 2991 cell processor, the Baxter CytoMate™, or the like. In some embodiments, the washed cells are resuspended in one or more biocompatible buffers or other saline solution with or without buffer. In certain embodiments, the undesired components of the apheresis sample are removed. Additional methods of isolating T cells for a T cell therapy are disclosed in U.S. Patent Publication No. 2013/0287748, which is herein incorporated by references in its entirety.

In certain embodiments, T cells are isolated from PBMCs by lysing the red blood cells and depleting the monocytes, e.g., by using centrifugation through a PERCOLL™ gradient. In some embodiments, a specific subpopulation of T cells, such as CD4⁺, CD8⁺, CD28⁺, CD45RA⁺, and CD45RO⁺ T cells is further isolated by positive or negative selection techniques known in the art. For example, enrichment of a T cell population by negative selection can be accomplished with a combination of antibodies directed to surface markers unique to the negatively selected cells. In some embodiments, cell sorting and/or selection via negative magnetic immunoadherence or flow cytometry that uses a cocktail of monoclonal antibodies directed to cell surface markers present on the cells negatively selected can be used. For example, to enrich for CD4⁺ cells by negative selection, a monoclonal antibody cocktail typically includes antibodies to CD8, CD1 lb, CD14, CD16, CD20, and HLA-DR. In certain embodiments, flow cytometry and cell sorting are used to isolate cell populations of interest for use in the present disclosure.

In some embodiments, PBMCs are used directly for genetic modification with the immune cells (such as CARs or TCRs) using methods as described herein. In certain embodiments, after isolating the PBMCs, T lymphocytes are further isolated, and both cytotoxic and helper T lymphocytes are sorted into naive, memory, and effector T cell subpopulations either before or after genetic modification and/or expansion.

In some embodiments, CD8⁺ cells are further sorted into naive, central memory, and effector cells by identifying cell surface antigens that are associated with each of these types of CD8⁺ cells. In some embodiments, the expression of phenotypic markers of central memory T cells includes CCR7, CD3, CD28, CD45RO, CD62L, and CD127 and are negative for granzyme B. In some embodiments, central memory T cells are CD8⁺, CD45RO⁺, and CD62L⁺ T cells. In some embodiments. effector T cells are negative for CCR7, CD28, CD62L, and CD127 and positive for granzyme B and perforin. In certain embodiments, CD4⁺ T cells are further sorted into subpopulations. For example, CD4⁺ T helper cells can be sorted into naive, central memory, and effector cells by identifying cell populations that have cell surface antigens.

In some embodiments, the immune cells, e.g., T cells, are genetically modified following isolation using known methods, or the immune cells are activated and expanded (or differentiated in the case of progenitors) in vitro prior to being genetically modified. In another embodiment, the immune cells, e.g., T cells, are genetically modified with the chimeric antigen receptors described herein (e.g., transduced with a viral vector comprising one or more nucleotide sequences encoding a CAR) and then are activated and/or expanded in vitro. Methods for activating and expanding T cells are known in the art and are described, e.g., in U.S. Pat. Nos. 6,905,874; 6,867,041; and 6,797,514; and PCT Publication No. WO 2012/079000. the contents of which are hereby incorporated by reference in their entirety. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). One example is The Dynabeads® system, a CD3/CD28 activator/stimulator system for physiological activation of human T cells. In other embodiments, the T cells are activated and stimulated to proliferate with feeder cells and appropriate antibodies and cytokines using methods such as those described in U.S. Pat. Nos. 6,040,177 and 5,827,642 and PCT Publication No. WO 2012/129514, the contents of which are hereby incorporated by reference in their entirety.

### Methods of Gene Delivery and Cell Modification

One of skill in the art would be well-equipped to construct a vector through standard recombinant techniques (see, for example, Sambrook et al., 2001 and Ausubel et al, 1996, both incorporated herein by reference) for the expression of the antigen receptors of the present disclosure. Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent. replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

### Viral Vectors

Viral vectors encoding an antigen receptor, a cytokine, and/or a functional effector element may be provided in certain aspects of the methods of the present disclosure. In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor mediated-endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present invention are described below.

In some embodiments of the methods of the disclosure, introducing a nucleic acid sequence and/or a genomic editing construct into an immune cell ex vivo, in vivo, in vitro or in situ comprises a viral vector. In some embodiments, the viral vector is a non-integrating non-chromosomal vector. Exemplary non-integrating, non-chromosomal vectors include, but are not limited to, adeno-associated virus (AAV), adenovirus, and herpes viruses. In some embodiments, the viral vector is an integrating chromosomal vector. Integrating chromosomal vectors include, but are not limited to, adeno-associated vectors (AAV), lentiviruses, and gamma-retroviruses.

In some embodiments of the methods of the disclosure, introducing a nucleic acid sequence and/or a genomic editing construct into an immune cell ex vivo, in vivo, in vitro, or in situ comprises a combination of vectors. Exemplary, non-limiting vector combinations include viral and non-viral vectors, a plurality of non-viral vectors, or a plurality of viral vectors. Exemplary but non-limiting vectors combinations include: a combination of a DNA-derived and an RNA-derived vector, a combination of an RNA and a reverse transcriptase, a combination of a transposon and a transposase, a combination of a non-viral vector and an endonuclease, and a combination of a viral vector and an endonuclease.

In some embodiments of the methods of the disclosure, genome modification comprising introducing a nucleic acid sequence and/or a genomic editing construct into an immune cell ex vivo, in vivo, in vitro or in situ stably integrates a nucleic acid sequence, transiently integrates a nucleic acid sequence, produces site-specific integration a nucleic acid sequence, or produces a biased integration of a nucleic acid sequence. In some embodiments, the nucleic acid sequence is a transgene.

In some embodiments of the methods of the disclosure, genome modification comprising introducing a nucleic acid sequence and/or a genomic editing construct into an immune cell ex vivo, in vivo, in vitro, or in situ stably integrates a nucleic acid sequence. In some embodiments, the stable chromosomal integration can be a random integration, a site-specific integration, or a biased integration. In some embodiments, the site-specific integration can be non-assisted or assisted. In some embodiments, the assisted site-specific integration is co-delivered with a site-directed nuclease. In some embodiments, the site-directed nuclease comprises a transgene with 5′ and 3′ nucleotide sequence extensions that contain a percentage homology to upstream and downstream regions of the site of genomic integration. In some embodiments, the transgene with homologous nucleotide extensions enables genomic integration by homologous recombination, microhomology-mediated end joining, or nonhomologous end-joining. In some embodiments the site-specific integration occurs at a safe harbor site. Genomic safe harbor sites are able to accommodate the integration of new genetic material in a manner that ensures that the newly inserted genetic elements function reliably (for example, are expressed at a therapeutically effective level of expression) and do not cause deleterious alterations to the host genome that cause a risk to the host organism. Potential genomic safe harbors include, but are not limited to, intronic sequences of the human albumin gene, the adeno-associated virus site 1 (AAVS1), a naturally occurring site of integration of AAV virus on chromosome 19, the site of the chemokine (C-C motif) receptor 5 (CCR5) gene and the site of the human ortholog of the mouse Rosa26 locus.

In some embodiments, the site-specific transgene integration occurs at a site that disrupts expression of a target gene. In some embodiments, disruption of target gene expression occurs by site-specific integration at introns, exons, promoters, genetic elements, enhancers, suppressors, start codons, stop codons, and response elements. In some embodiments, exemplary target genes targeted by site-specific integration include but are not limited to any immunosuppressive gene, and genes involved in allo-rejection.

In some embodiments, the site-specific transgene integration occurs at a site that results in enhanced expression of a target gene. In some embodiments, enhancement of target gene expression occurs by site-specific integration at introns, exons, promoters, genetic elements, enhancers, suppressors, start codons, stop codons, and response elements.

### Regulatory Elements

Expression cassettes included in vectors useful in the present disclosure in particular contain (in a 5′-to-3′ direction) a eukaryotic transcriptional promoter operably linked to a protein-coding sequence. splice signals including intervening sequences, and a transcriptional termination/polyadenylation sequence. The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters.

### Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively, a replication origin of other extra-chromosomally replicating virus as described above or an autonomously replicating sequence (ARS) can be employed.

### Other Methods of Nucleic Acid Delivery

In addition to viral delivery of the nucleic acids encoding the antigen receptor, the following are additional methods of recombinant gene delivery to a given cell, (e.g. an NK cell) and are thus considered in the present disclosure.

Introduction of a nucleic acid, such as DNA or RNA, into the immune cells of the current disclosure may use any suitable methods for nucleic acid delivery for transformation of a cell, as described herein or as would be known to one of ordinary skill in the art. Such methods include, but are not limited to, direct delivery of DNA such as by ex vivo transfection, by injection, including microinjection); by electroporation; by calcium phosphate precipitation; by using DEAE-dextran followed by polyethylene glycol; by direct sonic loading; by liposome mediated transfection and receptor-mediated transfection; by microprojectile bombardment; by agitation with silicon carbide fibers; by Agrobacterium-mediated transformation; by desiccation/inhibition-mediated DNA uptake. and any combination of such methods. Through the application of techniques such as these, organelle(s), cell(s), tissue(s) or organism(s) may be stably or transiently transformed.

Generally, the gene transfer system can include a transposon-based or a viral-based integration system.

In some embodiments, the gene transfer system comprises a transposon system. DNA transposons can translocate via a non-replicative “cut-and-paste” mechanism. This mechanism requires recognition of the two inverse terminal repeats (ITRs) by a catalytic enzyme, i.e., transposase, which can cleave its target and consequently release the DNA transposon from its donor template. Upon excision, the DNA transposons may subsequently integrate into the acceptor DNA that is cleaved by the same transposase. In some of their natural configurations, DNA transposons are flanked by two ITRs and may contain a gene encoding a transposase that catalyzes transposition. As would be appreciated by the skilled artisan, transposon systems offer many advantages for nucleic acid integration, e.g., as compared to viral vectors. For example. transposons can carry larger cargos, which can be advantageous for delivering one or more of the CARs, functional effector elements, and/or cytokines disclosed herein to an immune cell (e.g., an NK cell). Further, transposons may comprise, for example, CRISPR tools (e.g., along with cargo), and thereby allow multiplex engineering of a cell.

### Methods of Use

In some embodiments, the present disclosure provides methods for immunotherapy comprising administering an effective amount of the immune cells of the present disclosure. In one embodiment, a medical disease or disorder is treated by transfer of an immune cell population that elicits an immune response. In certain embodiments of the present disclosure, cancer or infection is treated by transfer of an immune cell population that elicits an immune response. Provided herein are methods for treating or delaying progression of cancer in an individual comprising administering to the individual an effective amount an antigen-specific cell therapy. The present methods may be applied for the treatment of immune disorders, solid cancers, hematologic cancers, and viral infections.

Tumors for which the present treatment methods are useful include any malignant cell type, such as those found in a solid tumor or a hematological tumor. In some embodiments, the cancer is acute myeloid leukemia. In some embodiments, the cancer is monocytic acute myeloid leukemia. In some embodiments, the cancer is mixed monocytic/primitive acute myeloid leukemia. In some embodiments, the cancer is a CD64-positive cancer. In some embodiments, the cancer has a low expression of CD64 (e.g. a CD64 low cancer). In some embodiments, the cancer has a low expression of CD64 and is converted to a CD64-positive cancer. In some embodiments, the cancer has a low expression of CD64 (e.g., a CD64 low cancer) prior to treatment with at least one acute myeloid leukemia-targeting treatment (e.g., treatment with venetoclax in combination with a hypomethylating agent, such as azacitidine) but the cancer remaining after treatment with at least one least one acute myeloid leukemia-targeting treatment has higher expression of CD64 (e.g., a CD64-positive cancer). In some embodiments, the cancer has little or no expression of CD64 and is converted to a CD64-positive cancer. In some embodiments, the cancer has little or no expression of CD64 (e.g., a CD64-negative cancer) prior to treatment with at least one acute myeloid leukemia-targeting treatment (e.g., treatment with venetoclax in combination with a hypomethylating agent, such as azacitidine), but the cancer remaining after treatment with at least one least one acute myeloid leukemia-targeting treatment expresses CD64 (e.g., a CD64-positive cancer).

Accordingly, the present disclosure provides methods of treating acute myeloid leukemia in a subject, the method comprising administering to the subject at least one amount of the immune cells of the present disclosure to the subject. The present disclosure provides the immune cells of the present disclosure for use in the treatment of acute myeloid leukemia in a subject. The present disclosure provides the use of the immune cells of the present disclosure in the manufacture of a medicament for the treatment of acute myeloid leukemia in a subject.

Particular embodiments concern methods of treatment of acute myeloid leukemia in a subject in need thereof. Acute myeloid leukemia (AML) is a blood cancer in which the bone marrow of a subject makes abnormal myeloblasts, red blood cells, or platelets. AML is one of the most common forms of acute leukemia in adults. The build-up of AML cells in bone marrow and blood can rapidly lead to infection, anemia, excessive bleeding and death. In some embodiments, the AML has a population of AML cells that are CD64+. In some embodiments, the AML lacks a population of AML cells that are CD64-positive prior to treatment with at least one AML-targeting therapy (e.g., treatment with venetoclax in combination with a hypomethylating agent, such as azacitidine), but the AML has a population of AML cells that are CD64-positive after treatment with at least one AML-targeting therapy. In some embodiments, the AML has a population of monocytic leukemia stem cells (mLSCs). In some embodiments, the subject has been previously administered at least one AML-targeting therapy. In some embodiments, the AML-targeting therapy comprises the administration of a combination of venetoclax and azacitidine. In some embodiments, the subject has relapsed after treatment with the at least one AML-targeting therapy and/or the subject is resistant to treatment with the at least one AML-targeting therapy. In some embodiments, the subject that has been previously administered at least one AML-targeting therapy (e.g., treatment with venetoclax in combination with a hypomethylating agent, such as azacitidine) has a population of AML cells that are CD64-positive, whereas the subject lacked a population of AML cells that were CD64-positive prior to the subject being administered at least one AML-targeting therapy.

In particular aspects, the CD64-specific CARs described herein are administrated to a subject having acute myeloid leukemia (AML). In particular aspects, the CD64-specific CARs described herein are administrated to a subject having a population of AML cells that are CD64+ and/or a population of monocytic leukemia stem cells (mLSCs). In particular aspects, the CD64-specific CARs described herein are administrated to a subject having relapsed after treatment with the at least one AML-targeting therapy and/or the subject is resistant to treatment with the at least one AML-targeting therapy. In particular aspects, the CD64-specific CARs described herein are administrated to a subject having a population of AML cells that are CD64-positive after treatment with at least one AML-targeting therapy (e.g., treatment with venetoclax in combination with a hypomethylating agent, such as azacitidine), whereas the subject lacked a population of AML cells that was CD64-positive prior to treatment with the at least one AML-targeting therapy.

In certain embodiments of the present disclosure, immune cells are delivered to an individual in need thereof, such as an individual that has cancer or an infection. The cells then enhance the individual's immune system to attack or directly attack the respective cancer or pathogenic cells. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, 8, 9, 10, 11, or 12 or more weeks.

In yet another embodiment, the subject is the recipient of a transplanted organ or stem cells, and immune cells are used to prevent and/or treat rejection. In particular embodiments, the subject has or is at risk of developing graft versus host disease. GVHD is a possible complication of any transplant that uses or contains stem cells from either a related or an unrelated donor. There are two kinds of GVHD, acute and chronic. Acute GVHD appears within the first three months following transplantation. Signs of acute GVHD include a reddish skin rash on the hands and feet that may spread and become more severe, with peeling or blistering skin. Acute GVHD can also affect the stomach and intestines, in which case cramping, nausea, and diarrhea are present. Yellowing of the skin and eyes (jaundice) indicates that acute GVHD has affected the liver. Chronic GVHD is ranked based on its severity: stage/grade 1 is mild; stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines. Any of the populations of immune cells disclosed herein can be utilized. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face. Immune cells can be administered prior to transplantation, concurrently with transplantation, or following transplantation. In some embodiments, the immune cells are administered prior to the transplant, such as at least 1 hour, at least 12 hours, at least 1 day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of immune cells occurs 3-5 days prior to transplantation.

In some embodiments, the subject can be administered nonmyeloablative lymphodepleting chemotherapy prior to the immune cell therapy. The nonmyeloablative lymphodepleting chemotherapy can be any suitable such therapy, which can be administered by any suitable route. The nonmyeloablative lymphodepleting chemotherapy can comprise, for example, the administration of venetoclax and azacitidine.

Therapeutically effective amounts of immune cells can be administered by a number of routes, including parenteral administration, for example, intravenous. intraperitoneal, intramuscular, intrasternal, or intraarticular injection, or infusion.

The therapeutically effective amount of immune cells for use in adoptive cell therapy is that amount that achieves a desired effect in a subject being treated. For instance, this can be the amount of immune cells necessary to inhibit advancement, or to cause regression of an autoimmune or alloimmune disease, or which is capable of relieving symptoms caused by an autoimmune disease, such as pain and inflammation. It can be the amount necessary to relieve symptoms associated with inflammation, such as pain, edema and elevated temperature. It can also be the amount necessary to diminish or prevent rejection of a transplanted organ. It can be the amount necessary to reduce the number of cancer cells in the subject. It can be the amount necessary to increase survival time of the subject.

The immune cell population can be administered in treatment regimens consistent with the disease, for example a single or a few doses over one to several weeks to ameliorate a disease state or periodic doses over an extended time to inhibit disease progression and prevent disease recurrence. The precise dose to be employed in the formulation will also depend on the route of administration, and the seriousness of the disease or disorder. The therapeutically effective amount of immune cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ immune cells/m². In a certain embodiment, the dose used in the treatment of human subjects ranges from about 3.8×10⁹ to about 3.8×10¹⁰ immune cells/m² (e.g., from about 3.8×10⁹ immune cells/m² to about 3.3×10¹⁰, from about 3.8×10⁹ to about 2.8×10¹⁰, from about 3.8×10⁹ to about 2.2×10¹⁰, from about 3.8×10⁹ to about 1.7×10¹⁰, from about 3.8×10⁹ to about 1.4×10¹⁰, from about 3.8×10⁹ to about 1×10¹⁰, from about 3.8×10⁹ to about 7.1×10⁹, from about 7.1×10⁹ to about 3.8×10¹⁰, from about 1×10¹⁰ to about 3.8×10¹⁰, from about 1.4×10¹⁰ to about 3.8×10¹⁰, from about 1.7×10¹⁰ to about 3.8×10¹⁰, from about 2.2×10¹⁰ to about 3.8×10¹⁰, from about 2.8×10¹⁰ to about 3.8×10¹⁰, from about 3.3×10⁹ to about 3.8×10¹⁰, from about 7.1×10⁹ to about 3.3×10¹⁰, from about 1×10¹⁰ to about 2.8×10¹⁰, or from about 1.4×10¹⁰ to about 2.2×10¹⁰). In additional embodiments, a therapeutically effective amount of immune cells can vary from about 5×10⁶ cells per kg body weight to about 7.5×10⁸ cells per kg body weight, such as from about 2×10⁷ cells to about 5×10⁸ cells per kg body weight, or from about 5×10⁷ cells to about 2×10⁸ cells per kg body weight, or from about 5×10⁶ cells per kg body weight to about 1×10⁷ cells per kg body weight. In some embodiments, a therapeutically effective amount of immune cells ranges from about 1×10⁵ cells per kg body weight to about 1×10⁹ cells per kg body weight (e.g., from about 1×10⁵ cells per kg body weight to about 8.8×10⁹. from about 1×10⁵ to about 7.5×10⁹, from about 1×10⁵ cells per kg body weight to about 6.3×10⁹, from about 1×10⁵ cells per kg body weight to about 5×10⁹, from about 1×10⁵ cells per kg body weight to about 3.8×10⁹, from about 1×10⁵ cells per kg body weight to about 2.5×10⁹, from about 1×10⁵ cells per kg body weight to about 1.3×10⁹, from about 1.3×10⁹ to about 1×10¹⁰, from about 2.5×10⁹ to about 1×10¹⁰, from about 3.8×10⁹ to about 1×10¹⁰, from about 5×10⁹ to about 1×10¹⁰, from about 6.3×10⁹ to about 1×10¹⁰, from about 7.5×10⁹ to about 1×10¹⁰, from about 1.3×10⁹ to about 8.8×10⁹, from about 2.5×10⁹ to about 7.5×10⁹, or from about 3.8×10⁹ to about 6.3×10⁹). The exact amount of immune cells is readily determined by one of skill in the art based on the age, weight, sex, and physiological condition of the subject. Effective doses can be extrapolated from dose-response curves derived from in vitro or animal model test systems.

The immune cells may be administered in combination with one or more other therapeutic agents for the treatment of the immune-mediated disorder. Combination therapies can include, but are not limited to, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetyls alicylic acid, ibuprofen or naproxen sodium), cytokine antagonists (for example, anti-TNF and anti-IL-6), cytokines (for example, interleukin-10 or transforming growth factor-beta), hormones (for example, estrogen), or a vaccine. In addition, immunosuppressive or tolerogenic agents including but not limited to calcineurin inhibitors (e.g., cyclosporin and tacrolimus); mTOR inhibitors (e.g., Rapamycin); mycophenolate mofetil, antibodies (e.g., recognizing CD3, CD4, CD40, CD154, CD45, IVIG, or B cells); chemotherapeutic agents (e.g., Methotrexate, Treosulfan, Busulfan); irradiation; chemokines, interleukins or their inhibitors (e.g., BAFF, IL-2, anti-IL-2R, IL-4, JAK kinase inhibitors); or immune checkpoint inhibitors (e.g., anti-PD1 antibodies, anti-PDL1 antibodies. anti-PDL2 antibodies, anti-LAG3 antibodies, and anti-CTLA4 antibodies) can be administered. Such additional pharmaceutical agents can be administered before, during, or after administration of the immune cells, depending on the desired effect. This administration of the cells and the agent can be by the same route or by different routes, and either at the same site or at a different site.

### Pharmaceutical Compositions

Also provided herein are pharmaceutical compositions and formulations comprising immune cells of the present disclosure (e.g., T cells) and a pharmaceutically acceptable carrier.

In some embodiments, a pharmaceutical composition comprises a dose ranging from about 1×10⁵ T cells to about 1×10⁹ T cells (e.g., from about 1×10⁵ T cells to about 8.75×10⁸, from about 5×10⁵ to about 7.5×10⁸, from about 5×10⁵ to about 6.25×10⁸, from about 5×10⁵ to about 5×10⁸, from about 5×10⁵ to about 3.75×10⁸, from about 5×10⁵ to about 2.5×10⁸, from about 5×10⁸ to about 1.25×10⁸, from about 1.25×10⁸ to about 1×10⁹, from about 2.5×10⁸ to about 1×10⁹, from about 3.75×10⁸ to about 1×10⁹, from about 5×10⁸ to about 1×10⁹, from about 6.25×10⁸ to about 1×10⁹, from about 7.5×10⁸ to about 1×10⁹, from about 8.75×10⁸ to about 1×10⁹, from about 1.25×10⁸ to about 8.75×10⁸, from about 2.5×10⁸ to about 7.5×10⁸, or from about 3.75×10⁸ to about 6.25×10⁸). In some embodiments, the dose is about 1×10⁵, 1×10⁶, 1×10⁷, 1×10⁸ or 1×10⁹ T cells. In some embodiments, a pharmaceutical composition comprises a dose ranging from about 5×10⁵ T cells to about 10×10¹² T cells (e.g., from about 5×10⁵ T cells to about 8.75×10¹¹, from about 5×10⁵ to about 7.5×10¹¹, from about 5×10⁵ to about 6.25×10¹¹, from about 5×10¹¹ to about 5×10¹¹, from about 5×10¹¹ to about 3.75×10¹¹, from about 5×10⁵ to about 2.5×10¹¹, from about 5×10⁵ to about 1.25×10¹¹, from about 1.25×10¹¹ to about 1×10¹², from about 2.5×10¹¹ to about 1×10¹², from about 3.75×10¹¹ to about 1×10¹², from about 5×10¹¹ to about 1×10¹², from about 6.25×10¹¹ to about 1×10¹², from about 7.5×10¹¹ to about 1×10¹², from about 8.75×10¹¹ to about 1×10¹², from about 1.25×10¹¹ to about 8.75×10¹¹, from about 2.5×10¹¹ to about 7.5×10¹¹, or from about 3.75×10¹¹ to about 6.25×10¹¹).

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

### Combination Therapies

In some embodiments, the compositions and methods of the present embodiments involve an immune cell population in combination with at least one additional therapy. The additional therapy may be radiation therapy, surgery (e.g., lumpectomy and a mastectomy), chemotherapy. gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy may be one or more of the chemotherapeutic agents known in the art.

An immune cell therapy may be administered before, during, after, or in various combinations relative to an additional cancer therapy. The administrations may be in intervals ranging from concurrently to minutes to days to weeks. Various combinations may be employed. For the example below an immune cell therapy is “A” and an anti-cancer therapy is “B”: A/B/A B/A/B B/B/A A/A/B A/B/B B/A/A A/B/B/B B/A/B/B B/B/B/A B/B/A/B A/A/B/B A/B/A/B A/B/B/A B/B/A/A B/A/B/A B/A/A/B A/A/A/B B/A/A/A A/B/A/A A/A/B/A

Administration of any compound or therapy of the present embodiments to a patient will follow general protocols for the administration of such compounds. taking into account the toxicity, if any, of the agents. Therefore, in some embodiments there is a step of monitoring toxicity that is attributable to combination therapy.

### Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclophosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, decitabine, dideoxyuridine, doxifluridine, enocitabine, and floxuridine; androgens. such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A. roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above. In some embodiments, azacitidine is administered at 75 mgs/m² subcutaneously.

In some aspects, the immune cells of the present disclosure can be administered in combination with venetoclax. In some aspects, the immune cells of the present disclosure can be administered in combination with azacitidine. In some aspects, the immune cells of the present disclosure can be administered in combination with venetoclax and azacitidine. In some aspects, the immune cells of the present disclosure can be administered after administration of venetoclax and/or azacitidine.

### Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287). and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

### Dosage Regimens

In one embodiment, the immune effector cells (e.g., T cells) are modified by engineering/introducing chimeric antigen receptors (e.g., anti-CD64 CAR) into said immune effector cells and then infused into a subject. In some embodiments, immune effector cells are modified by engineering/introducing a chimeric receptor, and functional effector element and/or a cytokine into the immune effector cells and then infused within about 0 days, within about 1 day, within about 2 days, within about 3 days, within about 4 days, within about 5 days, within about 6 days or within about 7 days into a subject.

In some embodiments, an amount of modified effector cells is administered to a subject in need thereof and the amount is determined based on the efficacy and the potential of inducing a cytokine-associated toxicity. In another embodiment, the modified effector cells are CAR⁺ and CD56⁺ cells. In some embodiments, an amount of modified effector cells comprises about 10⁴ to about 10⁹ modified effector cells/kg. In some cases, an amount of modified effector cells comprises about 10⁴ to about 10⁵ modified effector cells/kg. In some cases, an amount of modified effector cells comprises about 10⁵ to about 10⁶ modified effector cells/kg. In some cases, an amount of modified effector cells comprises about 10⁶ to about 10⁷ modified effector cells/kg. In some cases, an amount of modified effector cells comprises about 10⁷ to about 10⁸ modified effector cells/kg. In some cases, an amount of modified effector cells comprises about 10⁸ to about 10⁹ modified effector cells/kg. In some cases, am amount of modified effector cells comprises about 1×10⁶, about 2×10⁶. about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, about 1×10⁸, about 2×10⁸, about 3×10⁸, about 4×10⁸, about 5×10⁸, about 6×10⁸, about 7×10⁸, about 8×10⁸, about 9×10⁸, or about 1×10⁹ modified effector cells/kg.

In one embodiment, the modified immune effector cells are targeted to the cancer via regional delivery directly to the tumor tissue. For example, in ovarian or renal cancer, the modified immune effector cells can be delivered intraperitoneally (IP) to the abdomen or peritoneal cavity. Such IP delivery can be performed via a port or pre-existing port placed for delivery of chemotherapy drugs. Other methods of regional delivery of modified immune effector cells can include catheter infusion into resection cavity, ultrasound guided intratumoral injection, hepatic artery infusion or intrapleural delivery.

In one embodiment, a subject in need thereof, can begin therapy with a first dose of modified immune effector cells delivered via IV followed by a second dose of modified immune effector cells delivered via IV. In one embodiment, a subject in need thereof, can begin therapy with a first dose of modified immune effector cells delivered via IP followed by a second dose of modified immune effector cells delivered via IV. In a further embodiment, the second dose of modified immune effector cells can be followed by subsequent doses which can be delivered via IV or IP. In one embodiment, the duration between the first and second or further subsequent dose can be about: 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days. In one embodiment, the duration between the first and second or further subsequent doses can be about 0 days to about 30 days (e.g., about 0 days to about 27 days, about 0 to about 24, about 0 to about 21, about 0 to about 18, about 0 to about 15, about 0 to about 12, about 0 to about 9, about 0 to about 6, about 0 to about 3, about 3 to about 30, about 6 to about 30, about 9 to about 30, about 12 to about 30, about 15 to about 30, about 18 to about 30, about 21 to about 30, about 24 to about 30, about 27 to about 30, about 3 to about 27, about 6 to about 24, about 9 to about 21, about 12 to about 18, about 0 to about 6, about 3 to about 9, about 6 to about 12, about 9 to about 15, about 12 to about 18, about 15 to about 21, about 18 to about 24, about 21 to about 27, or about 24 to about 30. In one embodiment, the duration between the first and second or further subsequent dose can be about: 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months. In one embodiment, the duration between the first and second or further subsequent dose can be about 0 months to about 36 months (e.g., about 0 to about 30, about 0 to about 24, about 0 to about 18, about 0 to about 12, about 0 to about 6, about 6 to about 36, about 12 to about 36, about 18 to about 36, about 24 to about 36, about 30 to about 36, about 6 to about 30, about or 12 to about 24). In some embodiments, the duration between the first and second or further subsequent dose can be about: 0, 1, 2, 3, 4, 5, 6, 7, 8, 9 or 10 years. In some embodiments, the duration between the first and second or further subsequent dose can be about 0 years to about 10 years (e.g., about 0 years to about 9 years, about 0 to about 8, about 0 to about 7, about 0 to about 6, about 0 to about 5, about 0 to about 4, about 0 to about 3, about 0 to about 2, about 0 to about 1, about 1 to about 10, about 2 to about 10, about 3 to about 10, about 4 to about 10, about 5 to about 10, about 6 to about 10, about 7 to about 10, about 8 to about 10, about 9 to about 10, about 1 to about 9, about 2 to about 8, about 3 to about 7, about 4 to about 6, about 0 to about 2, about 1 to about 3, about 2 to about 4, about 3 to about 5, about 4 to about 6, about 5 to about 7, about 6 to about 8, about 7 to about 9, or about 8 to about 10).

In another embodiment, a catheter can be placed at the tumor or metastasis site for further administration of 1, 2, 3, 4, 5, 6, 7, 8, 9, 10 doses of modified immune effector cells. In some cases, doses of modified effector cells can comprise about 10² to about 10⁹ modified effector cells/kg (e.g., about 1×10² modified effector cells/kg to about 8.75×10⁸ effector cells/kg, about 1×10² to about 7.5×10⁸, about 1×10² to about 6.25×10⁸, about 1×10² to about 5×10⁸, about 1×10² to about 3.75×10⁸, about 1×10² to about 2.5×10⁸, about 1×10² to about 1.25×10⁸, about 1.25×10⁸ to about 1×10⁹, about 2.5×10⁸ to about 1×10⁹, about 3.75×10⁸ to about 1×10⁹, about 5×10⁸ to about 1×10⁹, about 6.25×10⁸ to about 1×10⁹, about 7.5×10⁸ to about 1×10⁹, about 8.75×10⁸ to about 1×10⁹, about 1.25×10⁸ to about 8.75×10⁸, about 2.5×10⁸ to about 7.5×10⁸, or about 3.75×10⁸ to about 6.25×10⁸). In cases where toxicity is observed, doses of modified effector cells can comprise about 10² to about 10⁵ modified effector cells/kg (e.g., about 1×10² modified effector cells/kg to about 7.5×10⁴ modified effector cells/kg, about 1×10² to about 5×10⁴, about 1×10² to about 2.5×10⁴, about 2.5×10⁴ to about 1×10⁵, about 5×10⁴ to about 1×10⁵, about 7.5×10⁴ to about 1×10⁵, about 2.5×10⁴ to about 7.5×10⁴, or about 4×10⁴ to about 6×10⁴. In some cases, doses of modified effector cells can start at about 10² modified effector cells/kg and subsequent doses can be increased to about: 10⁴, 10⁵, 10⁶, 10⁷, 10⁸ or 10⁹ modified effector cells/kg.

### Articles of Manufacture or Kits

An article of manufacture or a kit comprising immune cells is also provided herein. The article of manufacture or kit can further comprise a package insert comprising instructions for using the immune cells to treat or delay progression of cancer in an individual or to enhance immune function of an individual having cancer. Any of the antigen-specific immune cells described herein may be included in the article of manufacture or kits. Suitable containers include, for example, bottles, vials, bags, and syringes. The container may be formed from a variety of materials such as glass, plastic (such as polyvinyl chloride or poly olefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### General Definitions

As used herein, the term “portion” when used in reference to a polypeptide or a peptide refers to a fragment of the polypeptide or peptide. In some embodiments, a “portion” of a polypeptide or peptide retains at least one function and/or activity of the full-length polypeptide or peptide from which it was derived. For example, in some embodiments, if a full-length polypeptide binds a given ligand, a portion of that full-length polypeptide also binds to the same ligand.

The terms “protein” and “polypeptide” are used interchangeably herein.

The term “exogenous,” when used in relation to a protein, gene, nucleic acid, or polynucleotide in a cell or organism refers to a protein, gene, nucleic acid, or polynucleotide that has been introduced into the cell or organism by artificial or natural means: or in relation to a cell, the term refers to a cell that was isolated and subsequently introduced into a cell population or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from where it would be in natural cells or is otherwise flanked by a different nucleic acid sequence than that found in nature. The term “exogenous” is used interchangeably with the term “heterologous”.

By “expression construct” or “expression cassette” is used to mean a nucleic acid molecule that is capable of directing transcription. An expression construct includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included.

A “vector” or “construct” (sometimes referred to as a gene delivery system or gene transfer “vehicle”) refers to a macromolecule or complex of molecules comprising a polynucleotide, or the protein expressed by said polynucleotide, to be delivered to a host cell, either in vitro or in vivo.

A “plasmid,” a common type of a vector, is an extra-chromosomal DNA molecule separate from the chromosomal DNA that is capable of replicating independently of the chromosomal DNA. In certain cases, it is circular and double-stranded.

An “origin of replication” (“ori”) or “replication origin” is a DNA sequence, that when present in a plasmid in a cell is capable of maintaining linked sequences in the plasmid and/or a site at or near where DNA synthesis initiates. As an example, an ori for EBV (Ebstein-Barr virus) includes FR sequences (20 imperfect copies of a 30 bp repeat), and preferably DS sequences; however, other sites in EBV bind EBNA-1, e.g., Rep* sequences can substitute for DS as an origin of replication (Kirshmaier and Sugden, 1998). Thus, a replication origin of EBV includes FR. DS or Rep* sequences or any functionally equivalent sequences through nucleic acid modifications or synthetic combination derived therefrom. For example, methods of the present disclosure may also use genetically engineered replication origin of EBV, such as by insertion or mutation of individual elements.

A “gene,” “polynucleotide,” “coding region,” “sequence,” “segment,” “fragment,” or “transgene” that “encodes” a particular protein, is a section of a nucleic acid molecule that is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “control elements” refers collectively to promoter regions. polyadenylation signals, transcription termination sequences, upstream regulatory domains, origins of replication, internal ribosome entry sites (IRES), enhancers, splice junctions, and the like, which collectively provide for the replication, transcription, post-transcriptional processing, and translation of a coding sequence in a recipient cell. Not all of these control elements need be present so long as the selected coding sequence is capable of being replicated, transcribed, and translated in an appropriate host cell.

The term “promoter” is used herein to refer to a nucleotide region comprising a DNA regulatory sequence, wherein the regulatory sequence is derived from a gene that is capable of binding to a RNA polymerase and allowing for the initiation of transcription of a downstream (3′ direction) coding sequence. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

By “enhancer” is meant a nucleic acid sequence that, when positioned proximate to a promoter, confers increased transcription activity relative to the transcription activity resulting from the promoter in the absence of the enhancer domain.

By “operably linked” with reference to nucleic acid molecules is meant that two or more nucleic acid molecules (e.g., a nucleic acid molecule to be transcribed, a promoter, and an functional effector element) are connected in such a way as to permit transcription of the nucleic acid molecule. “Operably linked” with reference to peptide and/or polypeptide molecules means that two or more peptide and/or polypeptide molecules are connected in such a way as to yield a single polypeptide chain, i.e., a fusion polypeptide, having at least one property of each peptide and/or polypeptide component of the fusion. The fusion polypeptide is preferably chimeric, i.e., composed of molecules that are not found in a single polypeptide in nature.

The term “homology” refers to the percent of identity between the nucleic acid residues of two polynucleotides or the amino acid residues of two polypeptides. The correspondence between one sequence and another can be determined by techniques known in the art. For example, homology can be determined by a direct comparison of the sequence information between two polypeptides by aligning the sequence information and using readily available computer programs. Alternatively, homology can be determined by hybridization of polynucleotides under conditions that promote the formation of stable duplexes between homologous regions, followed by digestion with single strand-specific nuclease(s), and size determination of the digested fragments. Two polynucleotide (e.g., DNA), or two polypeptide, sequences are “substantially homologous” to each other when at least about 80%, at least about 90%, and most preferably at least about 95% of the nucleotides, or amino acids, respectively match over a defined length of the molecules, as determined using the methods above.

The term “cell” is herein used in its broadest sense in the art and refers to a living body that is a structural unit of tissue of a multicellular organism, is surrounded by a membrane structure that isolates it from the outside, has the capability of self-replicating, and has genetic information and a mechanism for expressing it. Cells used herein may be naturally-occurring cells or artificially modified cells (e.g., fusion cells, genetically modified cells, etc.).

The term “stem cell” refers herein to a cell that under suitable conditions is capable of differentiating into a diverse range of specialized cell types, while under other suitable conditions is capable of self-renewing and remaining in an essentially undifferentiated pluripotent state. The term “stem cell” also encompasses a pluripotent cell, multipotent cell, precursor cell and progenitor cell. Exemplary human stem cells can be obtained from hematopoietic or mesenchymal stem cells obtained from bone marrow tissue, embryonic stem cells obtained from embryonic tissue, or embryonic germ cells obtained from genital tissue of a fetus. Exemplary pluripotent stem cells can also be produced from somatic cells by reprogramming them to a pluripotent state by the expression of certain transcription factors associated with pluripotency; these cells are called “induced pluripotent stem cells” or “iPScs, “iPSCs” or “iPS cells”.

An “embryonic stem (ES) cell” is an undifferentiated pluripotent cell which is obtained from an embryo in an early stage, such as the inner cell mass at the blastocyst stage, or produced by artificial means (e.g., nuclear transfer) and can give rise to any differentiated cell type in an embryo or an adult, including germ cells (e.g., sperm and eggs).

“Induced pluripotent stem cells” (iPScs, iPSCs or iPS cells) are cells generated by reprogramming a somatic cell by expressing or inducing expression of a combination of factors (herein referred to as reprogramming factors). iPS cells can be generated using fetal, postnatal, newborn, juvenile, or adult somatic cells. In certain embodiments, factors that can be used to reprogram somatic cells to pluripotent stem cells include, for example, Oct4 (sometimes referred to as Oct 3/4), Sox2, c-Myc, Klf4, Nanog, and Lin28. In some embodiments, somatic cells are reprogrammed by expressing at least two reprogramming factors, at least three reprogramming factors, at least four reprogramming factors, at least five reprogramming factors, at least six reprogramming factors, or at least seven reprogramming factors to reprogram a somatic cell to a pluripotent stem cell.

“Hematopoietic progenitor cells” or “hematopoietic precursor cells” refers to cells which are committed to a hematopoietic lineage but are capable of further hematopoietic differentiation and include hematopoietic stem cells, multipotential hematopoietic stem cells, common myeloid progenitors, megakaryocyte progenitors, erythrocyte progenitors, and lymphoid progenitors. Hematopoietic stem cells (HSCs) are multipotent stem cells that give rise to all the blood cell types including myeloid (monocytes and macrophages, granulocytes (neutrophils, basophils, eosinophils, and mast cells), erythrocytes, megakaryocytes/platelets, dendritic cells), and lymphoid lineages (T-cells, B cells, NK cells) (see e.g., Doulatov et al., 2012; Notta et al., 2015).

A “multilymphoid progenitor” (MLP) is defined to describe any progenitor that gives rise to all lymphoid lineages (B. T. and NK cells), but that may or may not have other (myeloid) potentials (Doulatov et al., 2010) and is CD45RA⁺/CD10⁺/CD7⁺. Any B, T, and NK progenitor can be referred to as an MLP. A “common myeloid progenitor” (CMP) refers to CD45RA⁺/CD135⁺/CD10⁺/CD7⁺ cells that can give rise to granulocytes, monocytes, megakaryocytes and erythrocytes.

“Pluripotent stem cell” refers to a stem cell that has the potential to differentiate into all cells constituting one or more tissues or organs, or preferably, any of the three germ layers: endoderm (interior stomach lining, gastrointestinal tract, the lungs), mesoderm (muscle, bone, blood, urogenital), or ectoderm (epidermal tissues and nervous system).

As used herein, the term “somatic cell” refers to any cell other than germ cells, such as an egg, a sperm, or the like, which does not directly transfer its DNA to the next generation. Typically, somatic cells have limited or no pluripotency. Somatic cells used herein may be naturally-occurring or genetically modified.

“Programming” is a process that alters the type of progeny a cell can produce. For example, a cell has been programmed when it has been altered so that it can form progeny of at least one new cell type, either in culture or in vivo, as compared to what it would have been able to form under the same conditions without programming. This means that after sufficient proliferation, a measurable proportion of progeny having phenotypic characteristics of the new cell type are observed, if essentially no such progeny could form before programming; alternatively, the proportion having characteristics of the new cell type is measurably more than before programming. This process includes differentiation, dedifferentiation and transdifferentiation.

“Differentiation” is the process by which a less specialized cell becomes a more specialized cell type. “Dedifferentiation” is a cellular process in which a partially or terminally differentiated cell reverts to an earlier developmental stage, such as pluripotency or multipotency. “Transdifferentiation” is a process of transforming one differentiated cell type into another differentiated cell type. Typically, transdifferentiation by programming occurs without the cells passing through an intermediate pluripotency stage—i.e., the cells are programmed directly from one differentiated cell type to another differentiated cell type. Under certain conditions, the proportion of progeny with characteristics of the new cell type may be at least about 1%, 5%, 25% or more in order of increasing preference.

As used herein, the term “subject” or “subject in need thereof” refers to a mammal, preferably a human being, male or female at any age that is in need of a therapeutic intervention, a cell transplantation or a tissue transplantation. Typically, the subject is in need of therapeutic intervention, cell or tissue transplantation (also referred to herein as recipient) due to a disorder or a pathological or undesired condition, state, or syndrome. or a physical. morphological or physiological abnormality which is amenable to treatment via therapeutic intervention, cell or tissue transplantation.

As used herein, a “disruption” or “alteration” in reference to a gene refers to a homologous recombination event with a nucleic acid molecule (e.g., an endogenous gene sequence) which results in elimination or reduction of expression of one or more gene products encoded by the subject gene in a cell, compared to the level of expression of the gene product in the absence of the disruption. Exemplary gene products include mRNA and protein products encoded by the subject gene. Alteration in some cases is transient or reversible and in other cases is permanent. Alteration in some cases is of a functional or full-length protein or mRNA, despite the fact that a truncated or nonfunctional product may be produced. In some embodiments herein, gene activity or function, as opposed to expression, is disrupted. Gene alteration is generally induced by artificial methods, i.e., by addition or introduction of a compound, molecule, complex, or composition, and/or by alteration of nucleic acid of or associated with the gene, such as at the DNA level. Exemplary methods for gene alteration include gene silencing, knockdown, knockout, and/or gene alteration techniques. such as gene editing. Examples of gene editing methods include CRISPR/Cas systems, meganuclease systems, Zinc Finger Protein (ZFP) and Zinc Finger Nuclease (ZFN) systems and/or transcription activator-like protein (TAL), transcription activator-like effector protein (TALE) or TALE nuclease protein (TALEN) systems. Examples of gene alteration also include antisense technology, such as RNAi, siRNA, shRNA, and/or ribozymes, which generally result in transient reduction of expression, as well as gene editing techniques which result in targeted gene inactivation or alteration, e.g., by induction of breaks and/or homologous recombination. Examples include insertions, mutations, and deletions. The alterations typically result in the repression and/or complete absence of expression of a normal or “wild-type” product encoded by the gene. Exemplary of such gene alterations are insertions, frameshift and missense mutations, deletions, substitutions, knock-in, and knock-out of the gene or part of the gene, including deletions of the entire gene. Such alterations can occur in the coding region, e.g., in one or more exons, resulting in the inability to produce a full-length product, functional product, or any product, such as by insertion of a stop codon. Such alterations may also occur by alterations in the promoter or enhancer or other region affecting activation of transcription, so as to prevent transcription of the gene. Gene alterations include gene targeting, including targeted gene inactivation by homologous recombination.

An “immune disorder,” “immune-related disorder,” or “immune-mediated disorder” refers to a disorder in which the immune response plays a key role in the development or progression of the disease. Immune-mediated disorders include autoimmune disorders, allograft rejection, graft versus host disease and inflammatory and allergic conditions.

An “immune response” is a response of a cell of the immune system, such as a NK cell, B cell, or a T cell, or innate immune cell to a stimulus. In one embodiment, the response is specific for a particular antigen (an “antigen-specific response”).

As used herein, the term “antigen” is a molecule capable of being bound by an antibody, T cell receptor, Chimeric Antigen Receptor, and or engineered immune receptor. An antigen may generally be used to induce a humoral immune response and/or a cellular immune response leading to the production of B and/or T lymphocytes.

The terms “tumor-associated antigen,” “tumor antigen,” and “cancer cell antigen” are used interchangeably herein. In each case, the terms refer to proteins, glycoproteins, or carbohydrates that are specifically or preferentially expressed by cancer cells.

An “epitope” is the site on an antigen recognized by an antibody as determined by the specificity of the amino acid sequence. Two antibodies are said to bind to the same epitope if each competitively inhibits (blocks) binding of the other to the antigen as measured in a competitive binding assay. Alternatively, two antibodies bind to the same epitope if most amino acid mutations in the antigen that reduce or eliminate binding of one antibody reduce or eliminate binding of the other. Two antibodies are said to have overlapping epitopes if each partially inhibits binding of the other to the antigen, and/or if some amino acid mutations that reduce or eliminate binding of one antibody reduce or eliminate binding of the other.

An “autoimmune disease” refers to a disease in which the immune system produces an immune response (for example, a B cell or a T cell response) against an antigen that is part of the normal host (that is, an autoantigen), with consequent injury to tissues. An autoantigen may be derived from a host cell or may be derived from a commensal organism such as the micro-organisms (known as commensal organisms) that normally colonize mucosal surfaces.

The term “Graft-Versus-Host Disease (GVHD)” refers to a common and serious complication of bone marrow or other tissue transplantation wherein there is a reaction of donated immunologically competent lymphocytes against a transplant recipient's own tissue. GVHD is a possible complication of any transplant that uses or contains stem cells from either a related or an unrelated donor. In some embodiments, the GVHD is chronic GVHD (cGVHD).

A “parameter of an immune response” is any particular measurable aspect of an immune response, including, but not limited to, cytokine secretion (IFN-γ, etc.), chemokine secretion, altered migration or cell accumulation, immunoglobulin production, dendritic cell maturation, regulatory activity, number of immune cells and proliferation of any cell of the immune system. Another parameter of an immune response is structural damage or functional deterioration of any organ resulting from immunological attack. One of skill in the art can readily determine an increase in any one of these parameters, using known laboratory assays. In one specific non-limiting example, to assess cell proliferation, incorporation of ³H-thymidine can be assessed. A “substantial” increase in a parameter of the immune response is a significant increase in this parameter as compared to a control. Specific, non-limiting examples of a substantial increase are at least about a 50% increase, at least about a 75% increase, at least about a 90% increase, at least about a 100% increase, at least about a 200% increase, at least about a 300% increase, and at least about a 500% increase. Similarly, an inhibition or decrease in a parameter of the immune response is a significant decrease in this parameter as compared to a control. Specific, non-limiting examples of a substantial decrease are at least about a 50% decrease, at least about a 75% decrease, at least about a 90% decrease, at least about a 100% decrease, at least about a 200% decrease, at least about a 300% decrease, and at least about a 500% decrease. A statistical test, such as a non-parametric ANOVA, or a T-test, can be used to compare differences in the magnitude of the response induced by one agent as compared to the percent of samples that respond using a second agent. In some examples, p≤0.05 is significant, and indicates that the chance that an increase or decrease in any observed parameter is due to random variation is less than 5%. One of skill in the art can readily identify other statistical assays of use.

“Treating” or treatment of a disease or condition refers to executing a protocol or treatment plan, which may include administering one or more compositions to a patient (e.g. the anti-CD64 CAR compositions of the present disclosure), in an effort to alleviate signs or symptoms of the disease or the recurrence of the disease. Desirable effects of treatment include decreasing the rate of disease progression, ameliorating or palliating the disease state, and remission, increased survival, improved quality of life or improved prognosis. In addition, “treating” or “treatment” does not require complete alleviation of signs or symptoms, does not require a cure, and specifically includes protocols or treatment plans that have only a marginal effect on the patient.

As used herein, “prevent”, “preventing” and the like describe stopping the onset of the disease, condition or disorder, or one or more symptoms or complications thereof.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis or recurrence. Treatment of cancer may also refer to prolonging survival of a subject with cancer.

“Antibody” as used herein refers to monoclonal or polyclonal antibodies. The term “monoclonal antibodies,” as used herein, refers to antibodies that are produced by a single clone of B-cells and bind to the same epitope. In contrast, “polyclonal antibodies” refer to a population of antibodies that are produced by different B-cells and bind to different epitopes of the same antigen. A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CHL CH2 and CH3) regions, and each light chain contains one N-terminal variable (VL) region and one C-terminal constant (CL) region. The variable regions of each pair of light and heavy chains form the antigen binding site of an antibody. The VH and VL regions have a similar general structure, with each region comprising four framework regions, whose sequences are relatively conserved. The framework regions are connected by three complementarity determining regions (CDRs). The three CDRs, known as CDR1, CDR2, and CDR3, form the “hypervariable region” of an antibody, which is responsible for antigen binding.

“Antibody like molecules” may be for example proteins that are members of the Ig-superfamily which are able to selectively bind a partner.

The terms “fragment of an antibody,” “antibody fragment,”, “functional fragment of an antibody,” and “antigen-binding portion” are used interchangeably herein to mean one or more fragments or portions of an antibody that retain the ability to specifically bind to an antigen (see, generally, Holliger et al. (2005) Nat. Biotech. 23(9):1126-29). The antibody fragment desirably comprises, for example, one or more CDRs, the variable region (or portions thereof), the constant region (or portions thereof), or combinations thereof.

Examples of antibody fragments include, but are not limited to, (i) a Fab fragment, which is a monovalent fragment consisting of the VL, VH, CL, and CH1 domains: (ii) a F(ab′)2 fragment, which is a bivalent fragment comprising two Fab fragments linked by a disulfide bridge at the stalk region; (iii) a Fv fragment consisting of the VL and VH domains of a single arm of an antibody; (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Bird et al. (1988), Science 242: 423-6; Huston et al. (1988) Proc. Natl. Acad. Sci. USA 85: 5879-83; and Osbourn et al. (1998) Nat. Biotechnol. 16: 778-81) and (v) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites. Antibody fragments are known in the art and are described in more detail in, e.g., U.S. Patent Application Publication 2009/0093024 A1.

A “chimeric antigen receptor” is also known as an artificial cell receptor, a chimeric cell receptor. or a chimeric immunoreceptor. Chimeric antigen receptors (CARs) are engineered receptors, which graft a selected specificity onto an immune effector cell. CARs typically have an extracellular domain (ectodomain), which comprises an antigen-binding domain and a stalk region, a transmembrane domain and an intracellular (endodomain) domain.

A “stalk region”, which encompasses the terms “spacer region” or “hinge domain” or “hinge”, is used to link the antigen-binding domain to the transmembrane domain. As used herein, the term “stalk region” generally means any oligonucleotide or polypeptide that functions to link the transmembrane domain to, either the extracellular domain or, the cytoplasmic domain in the polypeptide chain of a CAR. In embodiments, it is flexible enough to allow the antigen-binding domain to orient in different directions to facilitate antigen recognition.

The term “functional portion,” when used in reference to a CAR, refers to any part or fragment of a CAR described herein, which part or fragment retains the biological activity of the CAR of which it is a part (the parent CAR). In reference to a nucleic acid sequence encoding the parent CAR, a nucleic acid sequence encoding a functional portion of the CAR can encode a protein comprising, for example. about 10%, 25%, 30%, 50%, 68%, 80%, 90%, 95%, or more, of the parent CAR.

The term “functional variant,” as used herein, refers to a polypeptide, or a protein having substantial or significant sequence identity or similarity to the reference polypeptide, and retains the biological activity of the reference polypeptide of which it is a variant. Functional variants encompass, for example, those variants of the CAR described herein (the parent CAR) that retain the ability to recognize target cells to a similar extent, the same extent, or to a higher extent, as the parent CAR. In reference to a nucleic acid sequence encoding the parent CAR, a nucleic acid sequence encoding a functional variant of the CAR can be for example, about 10% identical, about 25% identical, about 30% identical, about 50% identical, about 65% identical, about 70% identical, about 75% identical, about 80% identical, about 85% identical, about 90% identical, about 95% identical, or about 99% identical to the nucleic acid sequence encoding the parent CAR.

The phrases “pharmaceutical or pharmacologically acceptable” refers to molecular entities and compositions that do not produce an adverse, allergic, or other untoward reaction when administered to an animal, such as a human, as appropriate. For animal (e.g., human) administration, it will be understood that preparations should meet sterility, pyrogenicity, general safety, and purity standards as required, e.g., by the FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “T cell” refers to T lymphocytes, and includes, but is not limited to, γ/δ T cells, α/β T cells, NK T cells, CD4⁺ T cells and CD8⁺ T cells. CD4⁺ T cells include THO. Tₕ1 and TH2 cells, as well as regulatory T cells (T_{reg}). There are at least three types of regulatory T cells: CD4⁺ CD25⁺ T_{reg}, CD25 T_{H}3 T_{reg}, and CD25 T_{R} 1 T_{reg}. “Cytotoxic T cell” refers to a T cell that can kill another cell. The majority of cytotoxic T cells are CD8⁺ MHC class I-restricted T cells, however some cytotoxic T cells are CD4⁺. In some embodiments, the T cell of the present disclosure is CD4⁺ or CD8⁺.

The activation state of a T cell defines whether the T cell is “resting” (i.e., in the Gₒ phase of the cell cycle) or “activated” to proliferate after an appropriate stimulus such as the recognition of its specific antigen, or by stimulation with OKT3 antibody, PHA or PMA, etc. The “phenotype” of the T cell (e.g., naive, central memory, effector memory, lytic effectors, help effectors (THI and TH2 cells), and regulatory effectors), describes the function the cell exerts when activated. A healthy donor has T cells of each of these phenotypes, and which are predominately in the resting state. A naive T cell will proliferate upon activation, and then differentiate into a memory T cell or an effector T cell. It can then assume the resting state again, until it gets activated the next time, to exert its new function and may change its phenotype again. An effector T cell will divide upon activation and antigen-specific effector function.

“Natural killer T cells” (NKT cells), not to be confused with natural killer cells of the innate immune system, bridge the adaptive immune system with the innate immune system. Unlike conventional T cells that recognize peptide antigens presented by major histocompatibility complex (WIC) molecules, NKT cells recognize glycolipid antigen presented by a molecule called CD1d. Once activated, these cells can perform functions ascribed to both Th and Tc cells (i.e., cytokine production and release of cytolytic/cell killing molecules). They are also able to recognize and eliminate some tumor cells and cells infected with herpes viruses.

“Natural killer cells” (“NK cells”) are a type of cytotoxic lymphocyte of the innate immune system. In some instances. NK cells provide a first line defense against viral infections and/or tumor formation. NK cells can detect MHC presented on infected or cancerous cells, triggering cytokine release, and subsequently induce lysis and apoptosis. NK cells can further detect stressed cells in the absence of antibodies and/or MHC, thereby allowing a rapid immune response.

“Tumor antigen” as used herein refers to any antigenic substance produced, expressed or overexpressed in tumor cells. It may, for example, trigger an immune response in the host. Alternatively, for purposes of this disclosure, tumor antigens may be proteins that are expressed by both healthy and tumor cells but because they identify a certain tumor type, are a suitable therapeutic target. As described herein, in some aspects the tumor antigen can be CD64.

The term “antigen presenting cells (APCs)” refers to a class of cells capable of presenting one or more antigens in the form of peptide-MHC complex recognizable by specific effector cells of the immune system, and thereby inducing an effective cellular immune response against the antigen or antigens being presented. APCs can be intact whole cells such as macrophages, B cells, endothelial cells, activated T cells, and dendritic cells; or other molecules, naturally occurring or synthetic, such as purified MHC Class I molecules complexed to 2-microglobulin.

The term “culturing” refers to the in vitro maintenance, differentiation, and/or propagation of cells in suitable media. By “enriched” is meant a composition comprising cells present in a greater percentage of total cells than is found in the tissues where they are present in an organism.

An “anti-cancer” agent is capable of negatively affecting a cancer cell/tumor in a subject, for example, by promoting killing of cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

### EXAMPLES

### General Methods

### Generation of CD64 Chimeric Antigen Receptor Constructs

CD64-directed chimeric antigen receptors (CARs) were constructed utilizing single-chain fragment of the variable region (scFv's) which binds to human CD64. One version of this CAR consisted of the scFv joined to the extracellular domain of CD28, which was contiguous with the transmembrane domain and intracellular signaling domain of the CD28 protein. The CD28 signaling domain was joined with the intracellular signaling domain of the CD3-zeta chain and collectively this formed the CD64-28z CAR. Another version of this CAR consisted of the scFv joined to the extracellular domain of CD8, which was contiguous with the CD8 transmembrane domain. The CD8 transmembrane domain was joined to the intracellular signaling domain of the 4-1BB costimulatory receptor. The 4-1BB signaling domain was joined with the intracellular signaling domain of the CD3-zeta chain, collectively forming the CD64-BBz CAR.

### Generation of CD64 CAR T Cells

Lentivirus encoding the CAR transgene were produced through transient transfection of the Lenti-X 293T cell line (Takara Bio) with transfer plasmid and packaging plasmids (pRSV-Rev, pMDLg/pRRe and pMD2.G) using Lipofectamine 3000 (Life Technologies) in Opti-MEM (Thermo-Fisher). Media was replaced 6 hours after transfection and lentiviral supernatant was collected at 24- and 56-hours post-transfection, cleared by centrifugation at 2000 G for 10 minutes, aliquoted and frozen at −80° C._Human T cells were thawed at 0.5×10⁶ cells/mL and activated with a 3:1 ratio of Human T-expander CD3/CD28 Dynabeads per cell (Life Technologies) in AIM-V medium supplemented with 5% heat-inactivated FBS, 100 U/mL penicillin, 100 U/mL streptomycin, 5 mL GlutaMax™, 10 mM HEPES, and 40 IU/mL IL-2. T cells were transduced at an MOI of 2-10 by spinfection at 1000 g for 2 hours at 32° C. in the presence of 40 IU/mL IL-2 and 10 μg/mL protamine sulfate. Cells were incubated overnight at 37° C., and beads were removed the following day. CAR T cells were re-suspended at 0.5×10⁶/mL and expanded in vitro in 100 IU/mL of IL-2 for 4 days. CAR expression was verified by flow cytometry. CAR T cells were then cryopreserved 8 days post-activation. CAR T cells were thawed one day prior to start of in vitro and in vivo assays.

### In Vitro Testing of CD64 CAR T Cell Cytotoxicity Against AML Cell-Lines

The AML cell lines K562, MOLM14, and THP1 were stained with PE-conjugated anti-CD64 to demonstrate expression of CD64 on their surface. 10⁵ AML cell lines were co-incubated with 10⁵ CD64-28z CAR T cells or 10⁵ Mock T cells (which were activated and expanded but not transduced with the CAR). Co-incubations were carried out in 200 μL of media in wells of a 96-well plate. All co-incubations were performed in triplicate at 370 C, 10% CO₂ in a tissue culture incubator. 24 hours later, cells were collected from the co-incubations, stained with antibodies against CD3 and analyzed by flow cytometry. AML cells and T cells were distinguished from each other within the live cell gate based on CD3 staining (T cell marker) and either FSC (K562) or GFP expression (MOLM14 and THP1). Percentage of AML cell line within the live cell gate was quantified by flow cytometry and averaged across triplicate samples. Mann-Whitney Analysis was performed to determine statistical significance between CD64 CAR-treated and Mock T cell-treated groups.

### In Vitro Testing of CD64 CAR T Cell Cytokine Production in Response to AML Cell-Lines

10⁵ K562 or THP1 cells were co-incubated with 10⁵ CD64-28z CAR T cells or 10⁵ CD19-28z CAR T cells (an irrelevant CAR targeting an antigen not expressed on either cell line). Additionally, 10⁵ CD64-28z CAR T cells or 10⁵ CD19-28z CAR T cells were added to wells with media only as negative controls. Co-incubations were carried out in 200 μL of media in wells of a 96-well plate, in triplicate for 24 hours at 37° C., 8% CO₂ in a tissue culture incubator. At the end of co-incubation, cells were pelleted by centrifugation at 300 g for 6 minutes and then supernatant was collected from all wells and analyzed by ELISA (R&D systems) to quantify secreted Interferon-Gamma (IFNG) and Interleukin-2 (IL-2). Quantification of each cytokine was averaged across triplicate samples and Mann-Whitney Analysis was performed to determine statistical significance between CD64-28z CAR and CD19-28z CAR T cell groups.

### In Vivo Testing of CD64 CAR T Cells Against AML Cell-Lines

NOD/SCID/Gamma chain^{null} (NSG) immunodeficient mice were inoculated with 10⁶ luciferase-expressing THP1 or MOLM14 cells via tail vein injection on Day −4. Engraftment of the AML cells was confirmed by intraperitoneal (ip) injection of D-luciferin followed by BLI on Xenogen In Vivo Imaging System (IVIS) on Day −1. Mice were injected via tail vein with of 3-5×10⁶ CAR T cells or Mock T cells on Day 0. In some experiments, no treatment was administered on Day 0 as a negative control. At doses CD64-28z CAR T cells or were given no treatment. BLI was repeated once to twice weekly over the course of in vivo experiments to quantify leukemia burden. Mice were monitored 2-3 times per week for the development of predefined endpoint symptoms and were euthanized per institutional protocols upon reaching endpoint. Survival was recorded and statistically compared using Log-Rank Analysis.

### In Vitro Testing of CD64 CAR T Cells Against Patient-Derived AML Samples

Cryopreserved patient-derived AML samples acquired from the University of Colorado Biorepository were thawed and cell viability and concentration was determined by Trypan-Blue exclusion. 400,000 patient-derived AML cells were plated with CD64 CAR T cells or an equivalent number of Mock T cells. CAR T cells were added at varying E:T ratios. Co-incubation of CAR T cells and AML cells was carried out at 37° C., 10% CO₂ in a tissue culture incubator for 24-48 hours, after which the cells from the co-incubation were collected, washed, and stained for viability (Fixable Viability Dye eFlour780, Invitrogen) and with fluorescently-labeled antibodies against: CD45-PerCP-Cy5.5, CD3-BV605, CD34-BV421, and CD64-PE. Stained cells were analyzed on a Fortessa X-20 Flow cytometer (BD Biosciences). Live cells were analyzed for the ratio of AML cells (CD45^{dim}/CD3^{reg}) to T cells (CD45^{bright}/CD3^{pos}) and this ratio was normalized to mock-treated AML cells at each E:T ratio. AML cells were further analyzed for expression of CD34 and CD64. Statistical comparisons of Mock vs CAR (at either 24 and/or 48 hours) were performed using two-way ANOVA with Tukey's multiple comparison test.

### Ex Vivo Depletion of Leukemia Stem Cells in Patient-Derived AML Samples by CD64 CAR T Cells

Cryopreserved patient-derived AML samples acquired from the University of Colorado Biorepository were thawed and kept in culture at 37° C., 8% CO₂ in a tissue culture incubator. Cell viability and concentration was determined by trypan-blue exclusion. Patient-derived AML cells were co-incubated with CD64 CAR T cells at an E:T of 1:2 or an equivalent number of Mock T cells for 24 hours at 37° C., 10% CO₂ in a tissue culture incubator. T cells were depleted by antibody-mediated magnetic separation and 10⁶ AML cells were injected into NSG-S mice via tail vein. Mice were euthanized 6 weeks later. Bone marrow was collected from femurs, processed to a single cell suspension, red blood cells were lysed, and bone marrow cells were stained for viability (Fixable Viability Dye eFlour780, Invitrogen) and with fluorescently-labeled antibodies against: mouse CD45-BV786, human CD45-APC-R700, CD3-FITC, CD33-PE, and CD64-BV510. Human AML cells were defined as (mCD45^{reg}/hCD45^{pos}/CD3^{neg}/CD33^{pos}/CD64^{+/-}). Statistical comparisons between Mock and CAR T cell treatment were performed using Mann-Whitney Analysis.

### Example 1—CD64 CAR with a CD28 Costimulatory Domain (CD64-28z) T Cells Demonstrates Antigen-Dependent Effector Activity Against AML Cell Lines

CD64-28z-m22 CAR T cells, generated from healthy donor T cells, demonstrated the ability to target CD64+ AML cells lines in vitro after 24 hours co-incubation. Cytotoxicity of CD64-28z-m22 CAR T cells was evaluated by measuring residual AML cells by flow cytometry after co-incubation with either CAR T cells or Mock T cells. CD64-28z-m22 CAR T cells were able to significantly eliminate the CD64+ cell lines, MOLM14, and THP1 (FIG. 1A-B). Cytotoxicity was completely dependent upon CD64 antigen expression, as the CD64-negative cell line, K562, was not targeted by CD64-28z-m22 CAR T cells as demonstrated by no change in the amount of residual K562 cells after co-incubation with CAR T cells relative to Mock T cells (FIG. 1A-B). There was a subtle decrease in cytotoxicity of CD64-28z-m22 CAR T cells against the CD64-low MOLM14 cells relative to the CD64-high THP1 cells, further supporting the activity of CD64-28z-m22 CAR T cells is dependent upon antigen expression on the target cells (FIG. 1A-B). CD64-28z-m22 CAR T cells also secrete the effector cytokines Interferon-Gamma (IFNG) and Interleukin-2 (IL-2) in response to CD64+ cells (THP1), but not spontaneously (T cell only) or in response to CD64-negative (K562) cells (FIG. 1C). Without wishing to be bound by theory, these results demonstrate that the CD64-28z-m22 CAR is capable of recognizing CD64+ AML cells and responding through cytotoxicity and cytokine production.

### Example 2—CD64-28z-m22 CAR T Cells Demonstrate In Vivo Efficacy Against CD64+ AML Cells Lines in Xenograft Models

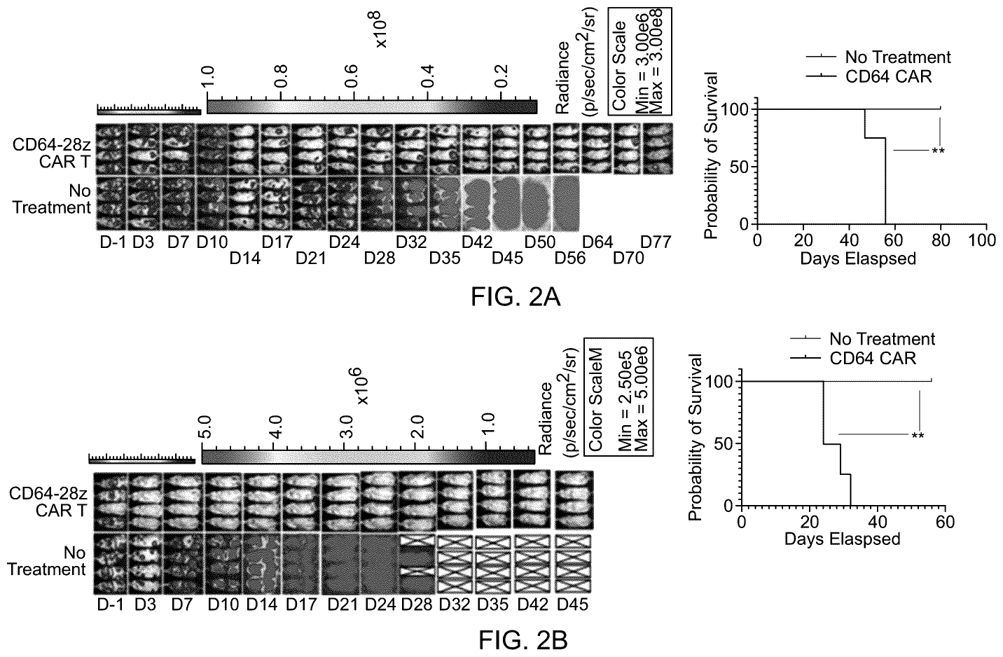

CD64-28z-m22 CAR T cells, generated from healthy donor T cells, demonstrate the ability to control AML progression in xenograft in vivo models. CD64-28z-m22 CAR T cells given to NSG mice engrafted with the CD64+ THP1 cell line demonstrated significant delay in leukemia progression and leukemia regression, ultimately significantly prolonging the survival of CAR-treated mice over untreated mice (FIG. 2A). Similarly, CD46-28z-m22 CAR T cells were able to eradicate CD64+ MOLM14 leukemia to undetectable levels and significantly prolonged the survival of mice beyond that of untreated mice (FIG. 2B).

### Example 3—CD64-28z-m22 CAR T Cells Demonstrate In Vitro Cytotoxicity Against Patient-Derived AML

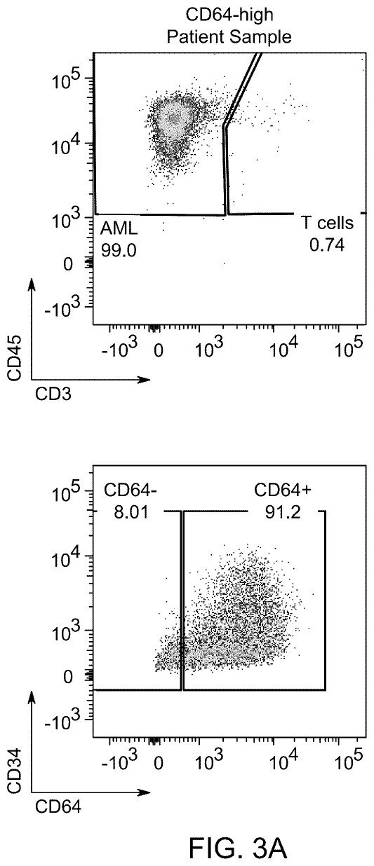

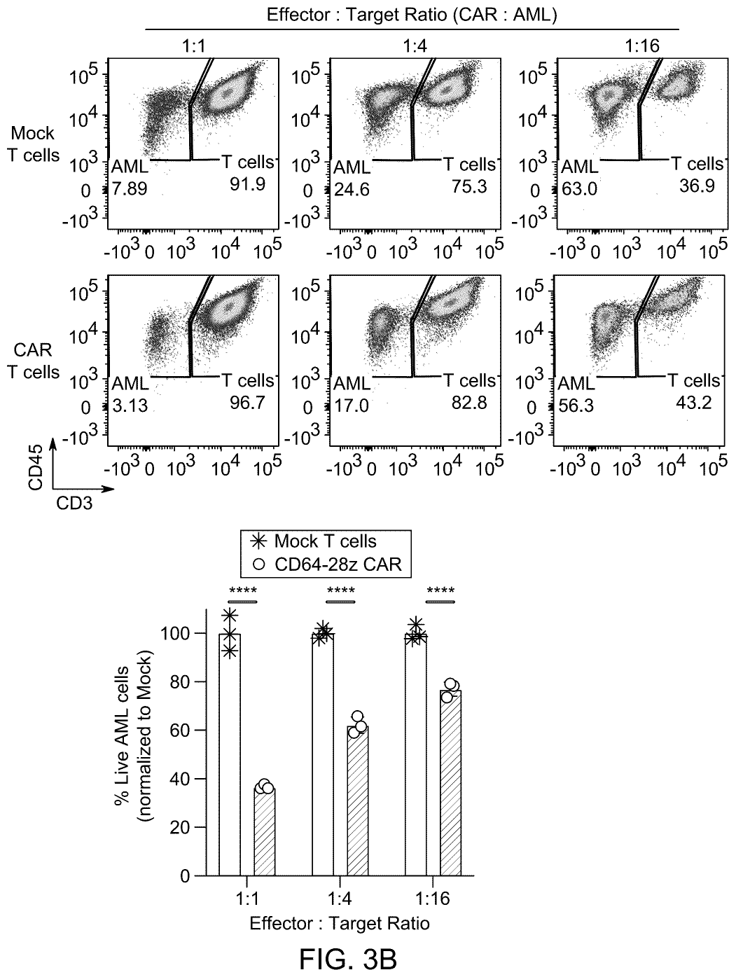

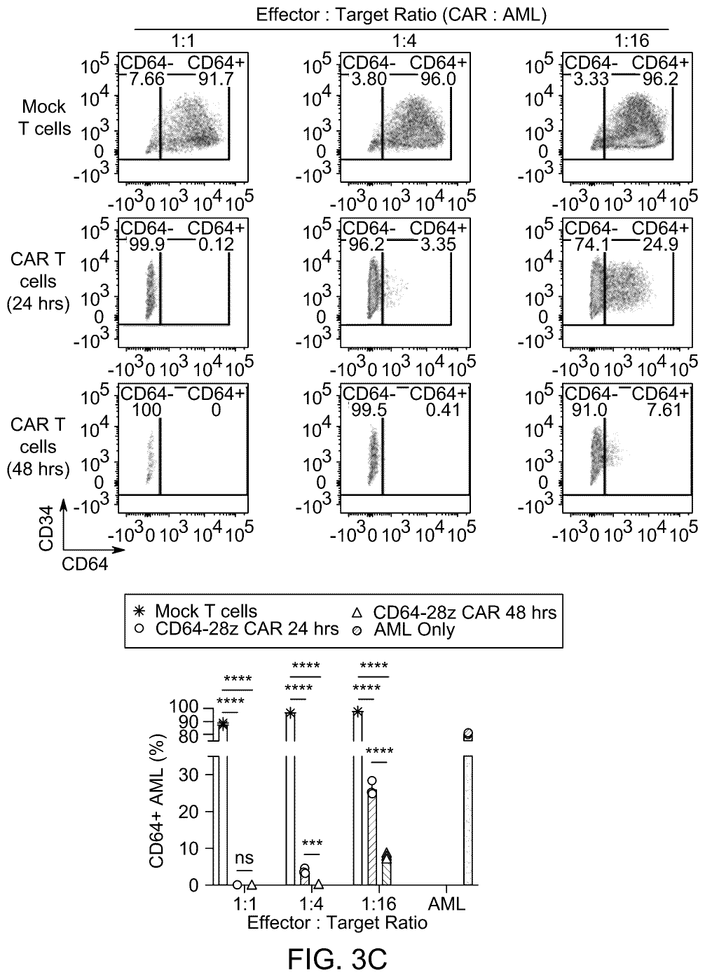

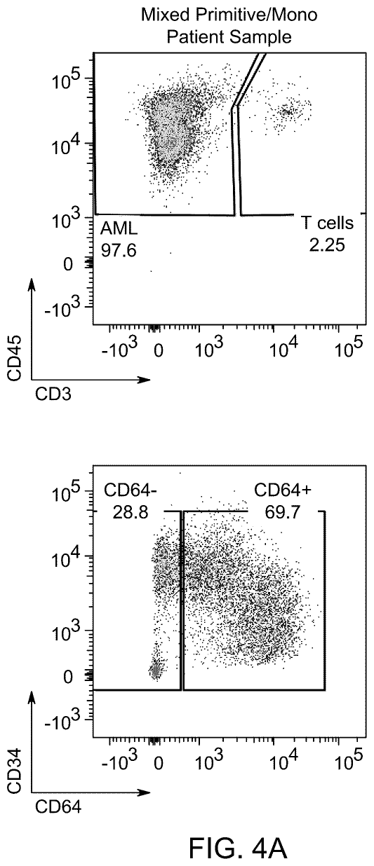

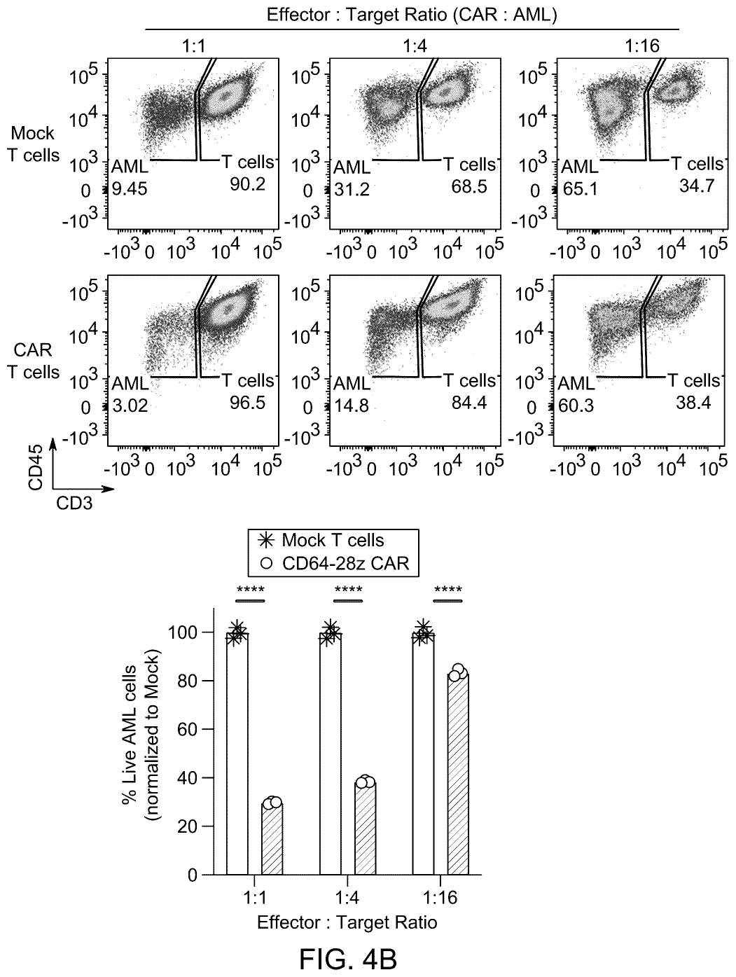

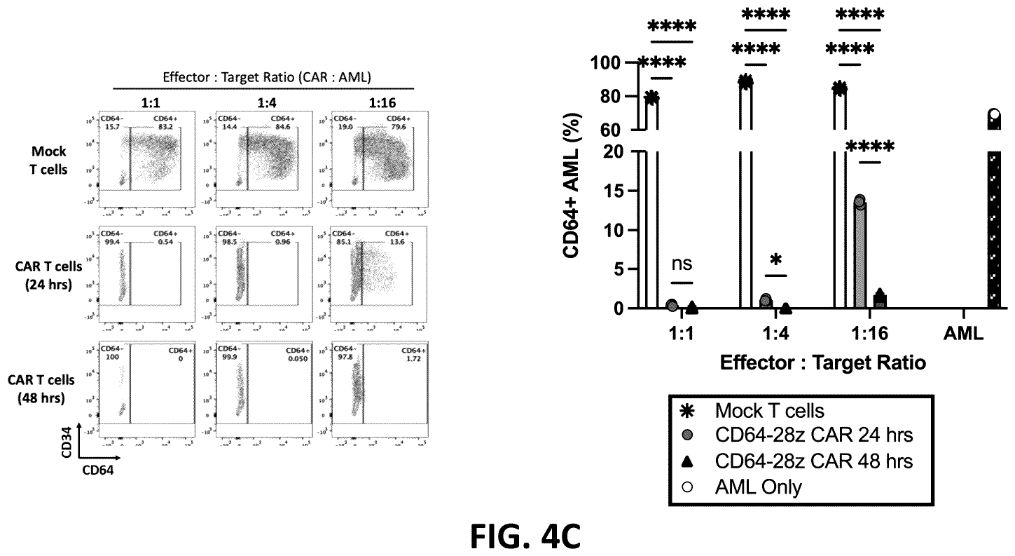

CD64-28z-m22 CAR T cells were tested for their ability to kill patient-derived AML that predominantly expressed CD64 (FIG. 3A). After 24 hours co-incubation, CD64-28z-m22 CAR T cells significantly reduced the amount of viable AML cells relative to mock T cells (FIG. 3B). In particular, CD64-28z-m22 CAR T cells eliminated the CD64+ cells within the AML, leading to virtually no detectable CD64+ at 24 hours at an E:T ratio of 1:1 and by 48 hours at the lower E:T of 1:4 (FIG. 3C). Even at E:T ratios as low as 1:16, there was significant reduction of both total AML and CD64+ AML at both 24 and 48 hours (FIG. 3B-C). Similar results were obtained when CD64-28z-m22 CAR T cells were co-incubated with patient-derived AML that contained both CD64+ and CD64− subpopulations (FIG. 4A) stemming from primitive (CD64−) and monocytic (CD64+) leukemia stem cell (LSC) populations, as previously described by Pei et. al., Cancer Discovery, 2020; 10(4):536-551. CD64-28z-m22 CAR T cells significantly reduced the number of viable AML cells in 24 hours, relative to Mock T cells (FIG. 4B). CD64-28z-m22 CAR T cells were found to also significantly deplete the CD64+ AML cells, with near complete elimination of CD64+ AML at 24 hours at E:T ratios of 1:1 and 1:4 and of E:T ratios as low as 1:16 at 48 hours (FIG. 4C). Without wishing to be bound by theory, these results demonstrate that the CD64-28z-m22 CAR shows effective cytotoxicity against patient-derived AML as well as AML cell lines (as demonstrated in FIG. 1A-B).

### Example 4—CD64-28z-m22 CAR T Cells Deplete Monocytic Leukemic Stem Cells (mLSCs) from Patient-Derived AML Samples

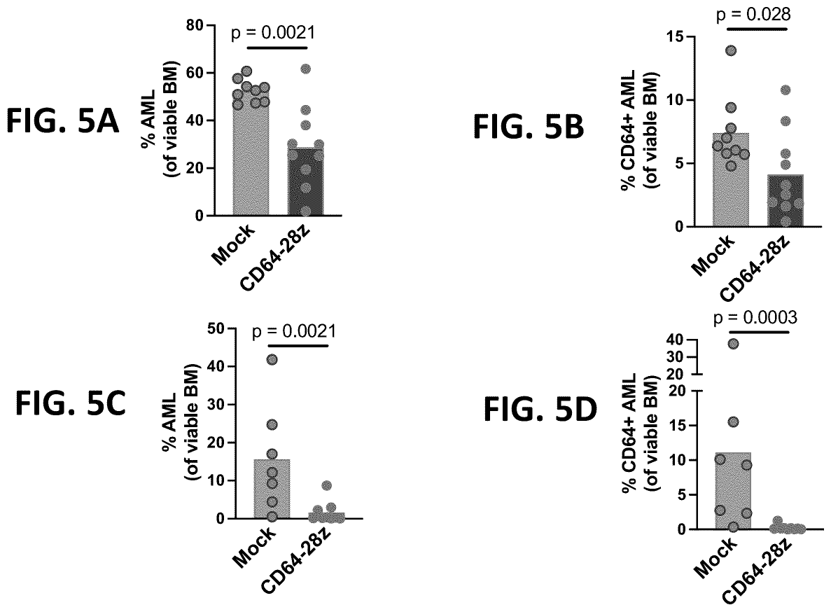

As monocytic leukemia stem cells (mLSCs) have been reported to drive resistance to common AML therapies, such as venetoclax and azacytidine, in patients (Pei et. al., Cancer Discovery, 2020; 10(4):536-551), the CD64-28z-m22 CAR was tested for its ability to specifically deplete mLSCs from patient-derived AML samples. As in vivo engraftment is the primary test of LSC activity, patient derived AML samples were first co-incubated with CD64-28z-m22 CAR T cells or Mock T cells at an E:T of 1:2 for 24 hours to allow for CAR T cell killing of the mLSC population. After 24 hours, T cells were depleted, and the remaining AML cells were inoculated into immunocompromised NSG-S mice. Leukemia engraftment was evaluated 6 weeks later and found to be significantly impaired after CAR T cell co-incubation relative to co-incubation with Mock T cells (FIG. 5A-D). Pre-treatment of the AML with CD64-28z-m22 CAR T cells resulted in decreased engraftment of the total AML population and the CD64+ AML population of patient-derived AML that was predominantly CD64+(FIG. 5A-B), suggesting the depletion of mLSCs prior to inoculation into the mice. Similarly, there was decreased engraftment of the total AML population and the CD64+ subpopulation of AML from a patient with mixed primitive/monocytic AML after co-incubation with CD64-28z-m22 CAR T cells (FIG. 5C-D). Without wishing to be bound by theory, these results demonstrate that CD64 CAR T cells can specifically deplete the monocytic leukemia stem cell population that has been implicated in patient relapses, particularly after treatment with venetoclax and azacitidine.

### Example 5—CD64-BBz-m22 and CD64-28z-m22 CAR T Cells Demonstrate In Vivo Efficacy Against CD64+ AML Cells Lines in Xenograft Models

CD64 CARs containing either a CD28 costimulatory domain (CD64-28z-m22) or a 4-1BB costimulatory domain (CD64-BBz-m22) were generated from T cells of two independent healthy donors and were tested for their ability to control the CD64+ MOLM14 AML cell line in xenograft models. Both CD64-28z-m22 and CD64-BBz-m22 CAR T cells significantly slowed leukemia progression and/or eliminated leukemia compared to Mock T cell treatment (FIG. 6A). The efficacy of CD64 CAR T cells was seen when CAR T cells were generated from different healthy T cell donors (FIG. 6A) and both CD64-28z-m22 and CD64-BBz-m22 CAR T cells significantly prolonged the survival of AML-bearing mice (FIG. 6B). Quantification of total bioluminescent flux per mouse demonstrated decreased leukemia burden in mice treated with CD64-28z-m22 and CD64-BBz-m22 CAR T cells as compared to mock treated mice (FIG. 6C). Without wishing to be bound by theory, these results demonstrate a significant survival advantageous of CD64-BBz-m22 CAR T cells relative to CD64-28z-m22 CAR T cells (FIG. 6B).

### Example 6—CD64 CAR T Cells Demonstrate Efficacy Against Monocytic and Mixed Monocytic/Primitive AML

The results in this Example re-present and expand on at least some of the results provided in other Examples.

### CD64 Expression in AML Models

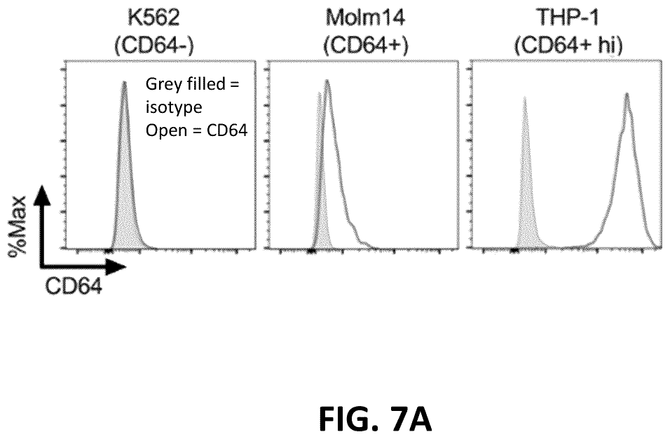

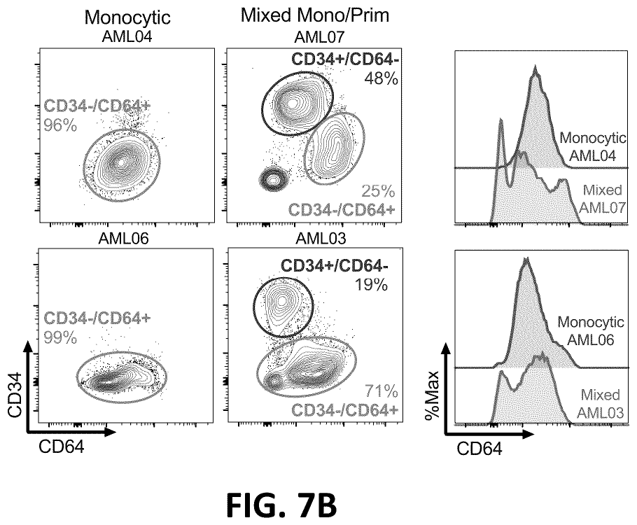

In order to assess the preclinical efficacy of the CD64 CAR, a variety of target leukemia models with varying levels of CD64 expression were utilized. AML cell lines with high, moderate, and negative CD64 expression to test CD64 CAR efficacy were employed. CD64 expression in three model cell lines was compared using flow cytometry with staining for CD64 (FIG. 7A). The CD64+ cell line MOLM14 expressed a low-moderate level of CD64. The monocytic AML cell line, THP-1, expressed high levels of CD64. The K562 cell line was used as a negative control as it did not express CD64.

Primary AML samples with differing phenotypes, including monocytic (FAB M5) patient samples (AML04 and AML06 with uniformly high CD64 expression (CD64bright, CD34dim/−)) and mixed monocytic and primitive AML patient samples (AML03 and AML07, (CD64dim/−, CD34bright)), were used (FIG. 7B).

### CD64 CAR Expression in Donor T-Cells

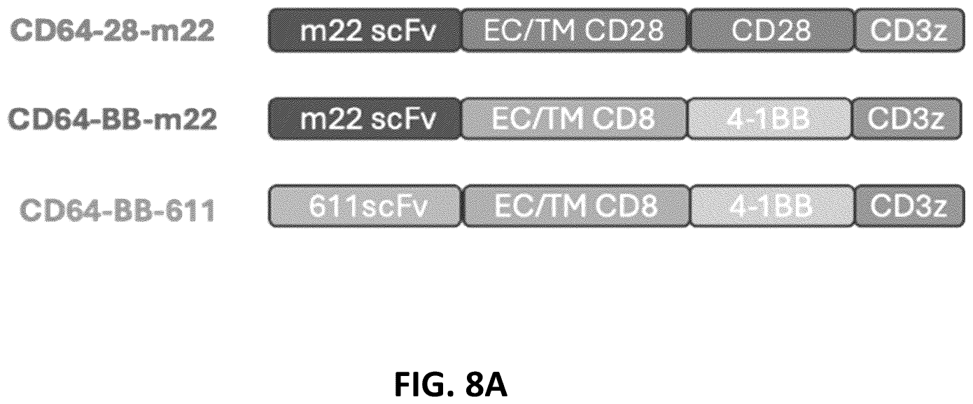

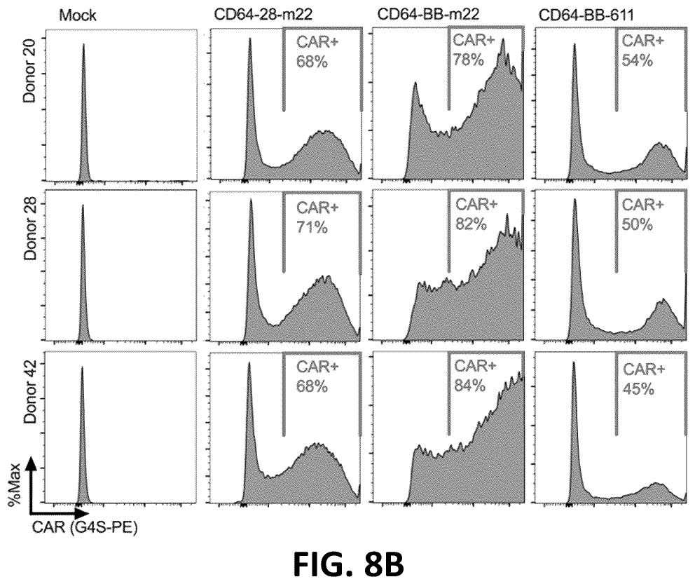

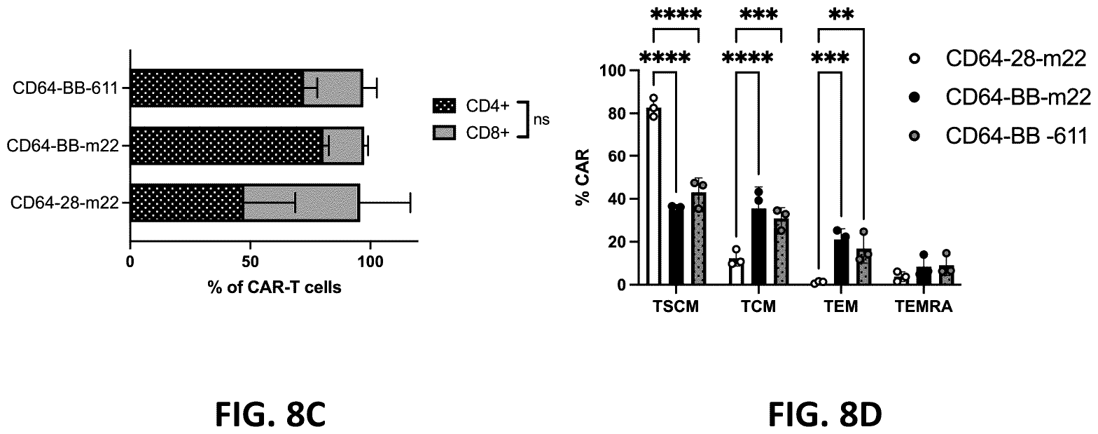

Human T cells were transduced with the CD64 CAR transgenes by lentiviral transduction following T cell activation with anti-CD3/CD28 T-Expander Dynabeads™ (Invitrogen) in the presence of IL-2 (R&D, 40 IU/mL). After transduction, the anti-CD3/CD28 Dynabeads™ were removed and T cells were expanded in the presence of IL-2 (100 IU/mL) for 5 days. Three different CAR constructs were tested. CD64-28-m22 included a scFv derived from the murine M22 antibody (which targets human CD64) attached to a portion of the extracellular domain of human CD28, the human CD28 transmembrane domain, and the human CD28 intracellular signaling domain inline with the human CD3-zeta signaling domain with all three ITAMs intact (FIG. 8A). CD64-BB-m22 included the same M22 scFv attached to a portion of the extracellular domain of human CD8a and the human CD8a transmembrane domain. This transmembrane domain was inline with the intracellular signaling domain of human 4-1BB and human CD3-zeta with all three ITAMs intact. CD64-BB-611 incorporated a scFv derived from the humanized anti-CD64 antibody, 611, attached to a portion of the extracellular domain of human CD8a and the human CD8a transmembrane domain. This transmembrane domain was in line with the intracellular signaling domain of human 4-1BB and human CD3-zeta with all three ITAMs intact.

Healthy human donor T cells were successfully transduced with all CD64 CAR constructs. CAR molecules were consistently expressed across T cells from multiple healthy donors, as measured by flow cytometry using antibodies recognizing the G4S (SEQ ID NO: 422) linker of the CAR scFvs (FIG. 8B). In CAR+ populations transduced with a CAR construct incorporating a 4-1BB signaling domain, there was a skewing to a higher ratio of CD4 to CD8 T cells. There was a more balanced ratio of CD4 to CD8 seen in CAR+ populations transduced with a CAR incorporating the CD28 costimulatory domain (FIG. 8C).

It was found that T cells transduced the CD64-28-m22 CAR predominantly had a phenotype consistent with T stem cell memory (TSCM) cells (CD62L+/CD45RA+/CD95+), whereas T cells transduced with either CD64-BB-m22 or CD64-BB-611 demonstrated a mixture of phenotypes consistent with TSCM, T central memory (TCM, CD62L+/CD45RA−/CD95+), and T effector memory (TEM, CD62L−/CD45RA−/CD95+) (FIG. 8D). These phenotypic trends were consistent across T cells from multiple healthy donors. Statistical analysis of three independent donors by two-way ANOVA with Turkey's multiple comparison test was performed.

### CD64 CAR T Cell Cytotoxicity In Vitro

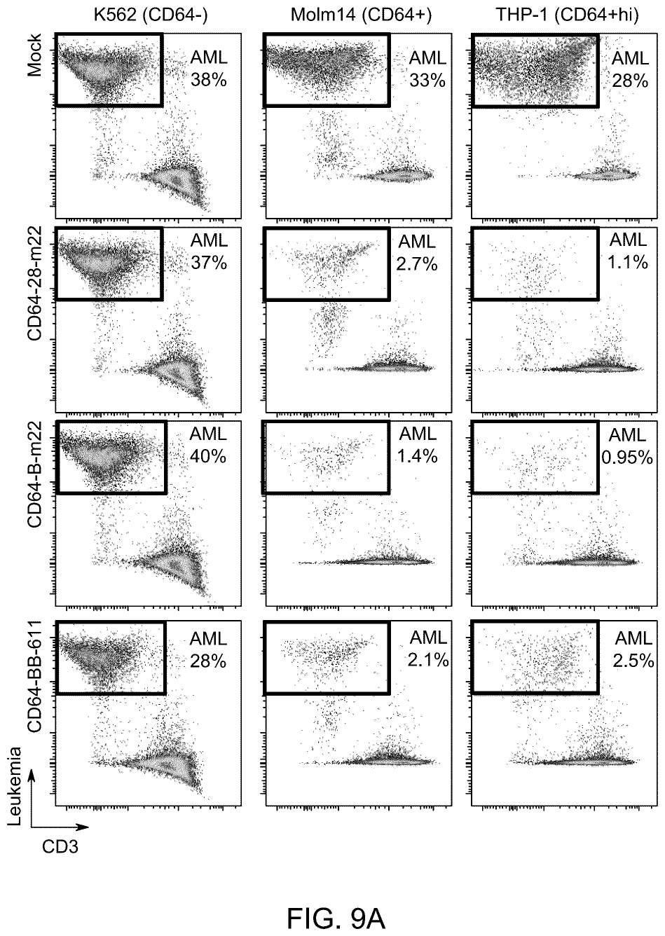

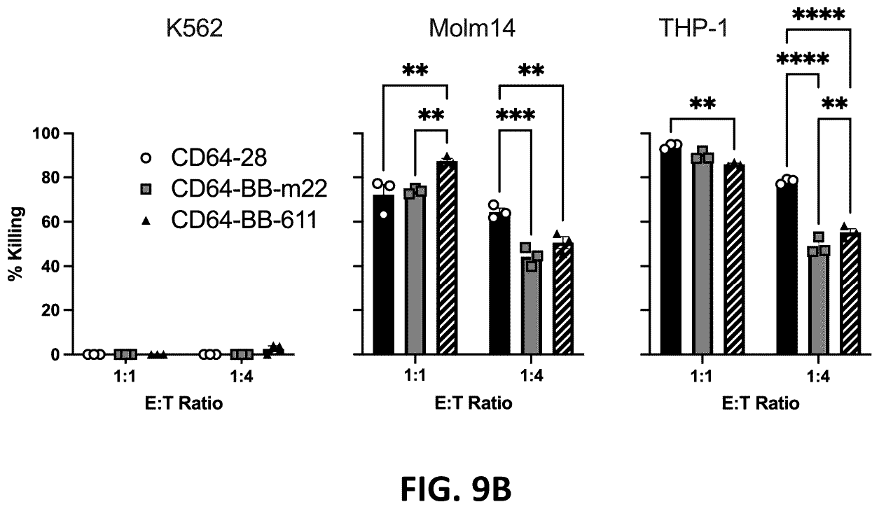

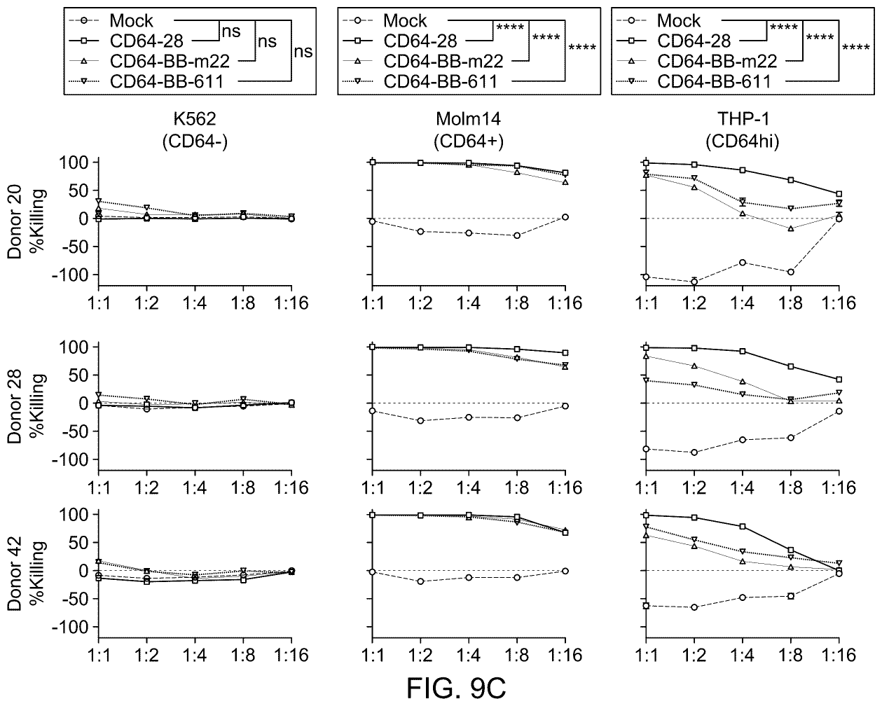

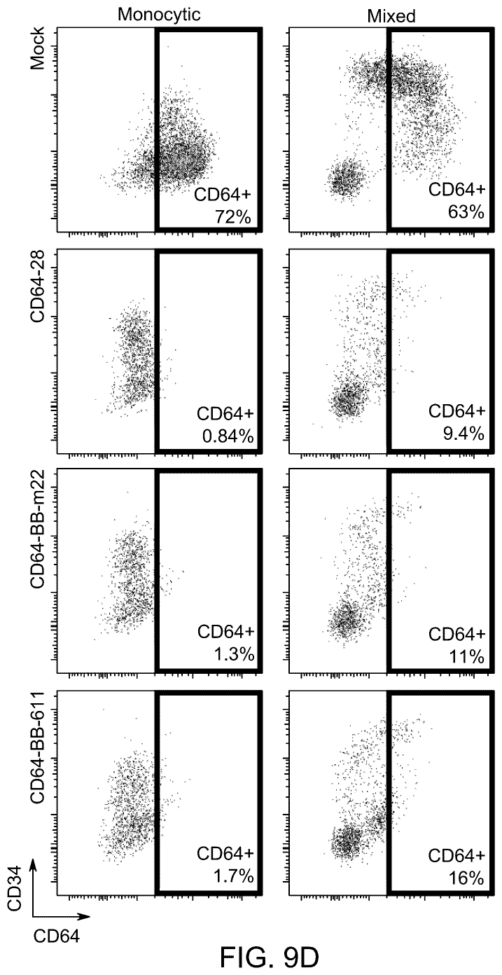

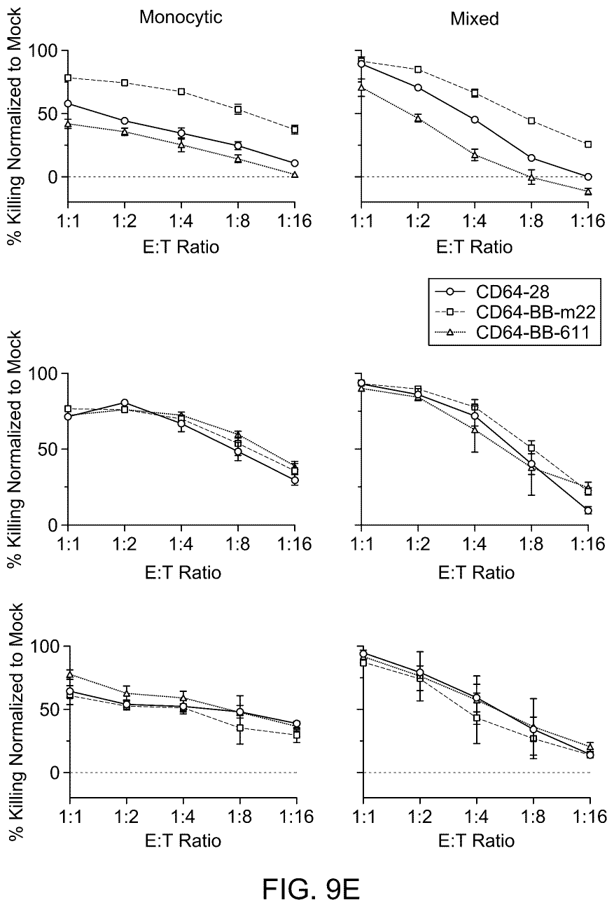

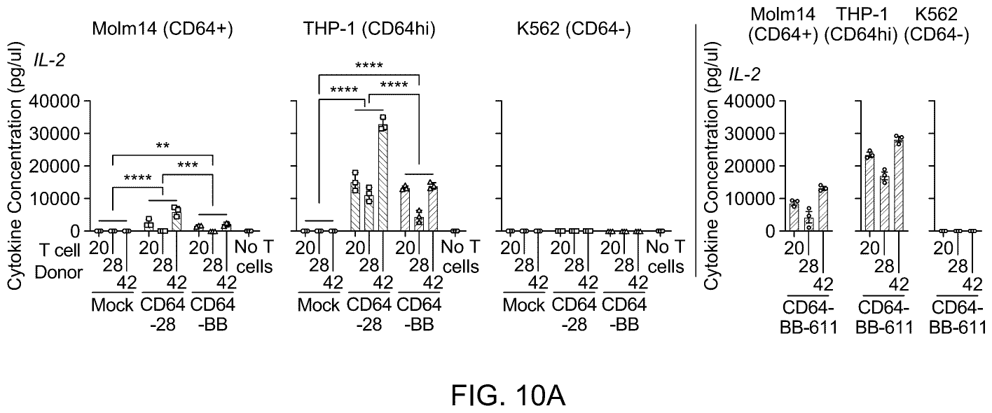

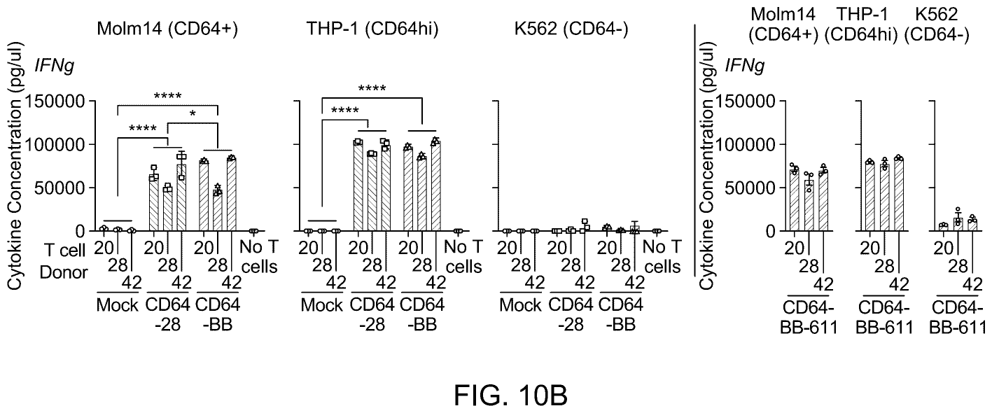

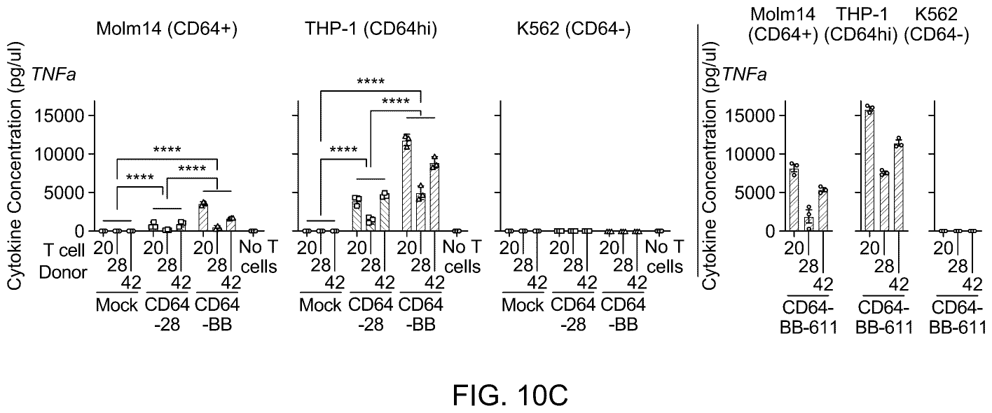

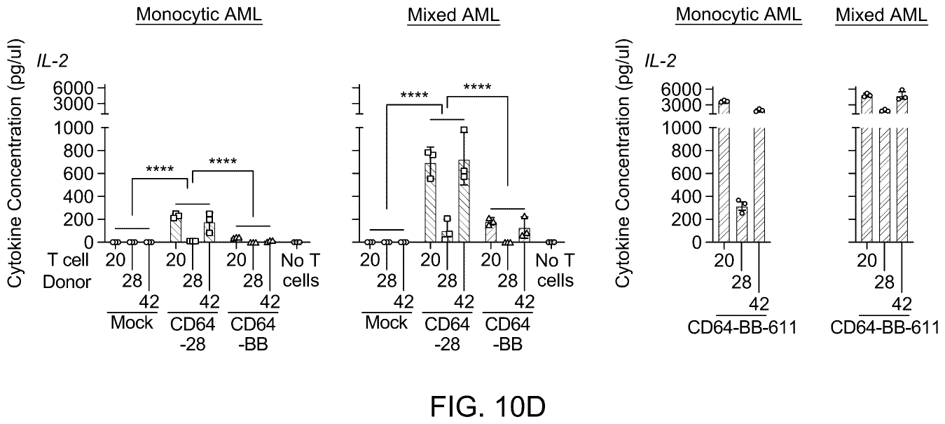

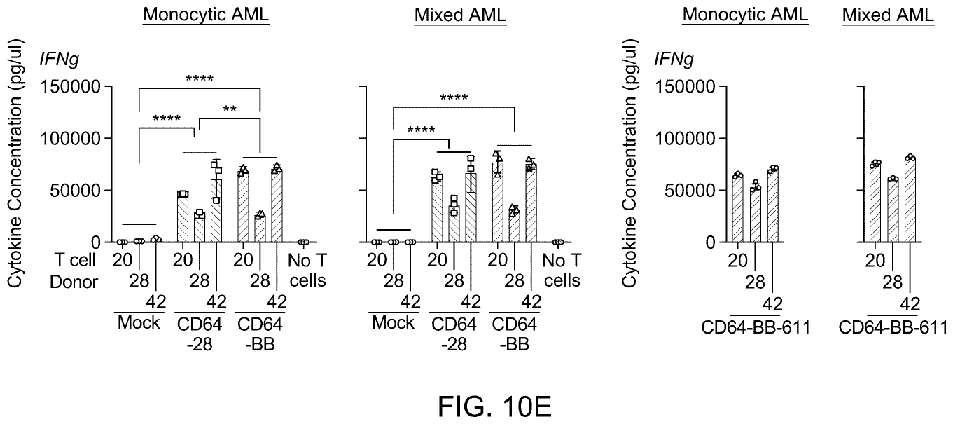

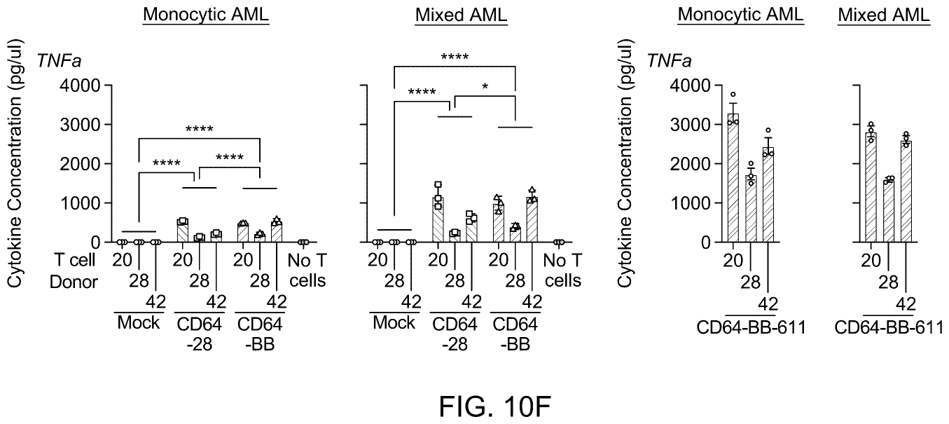

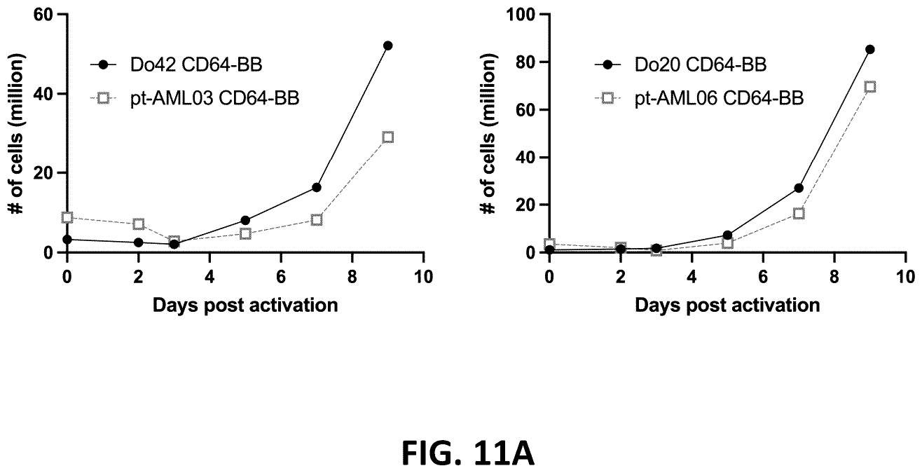

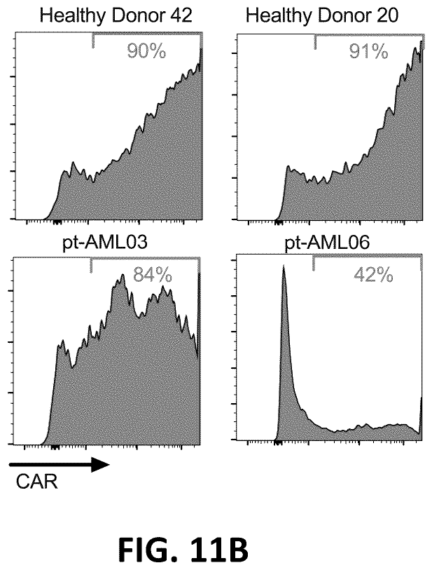

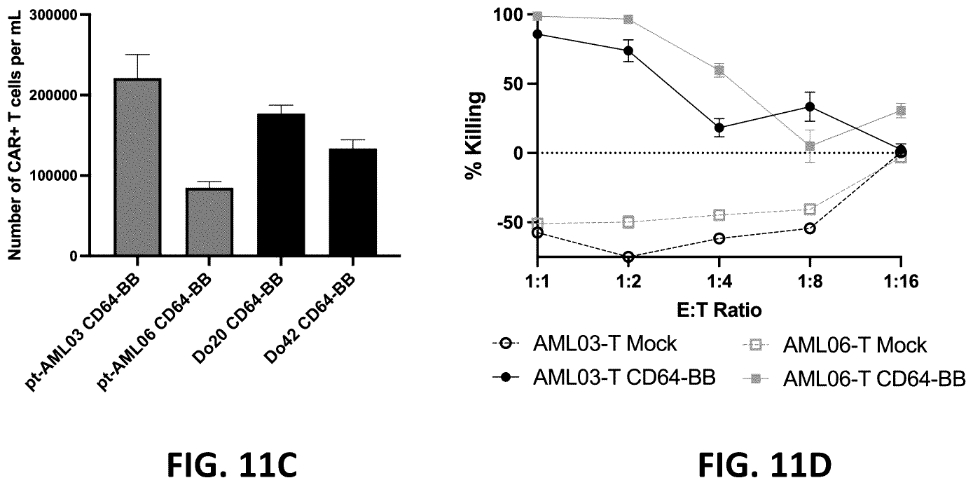

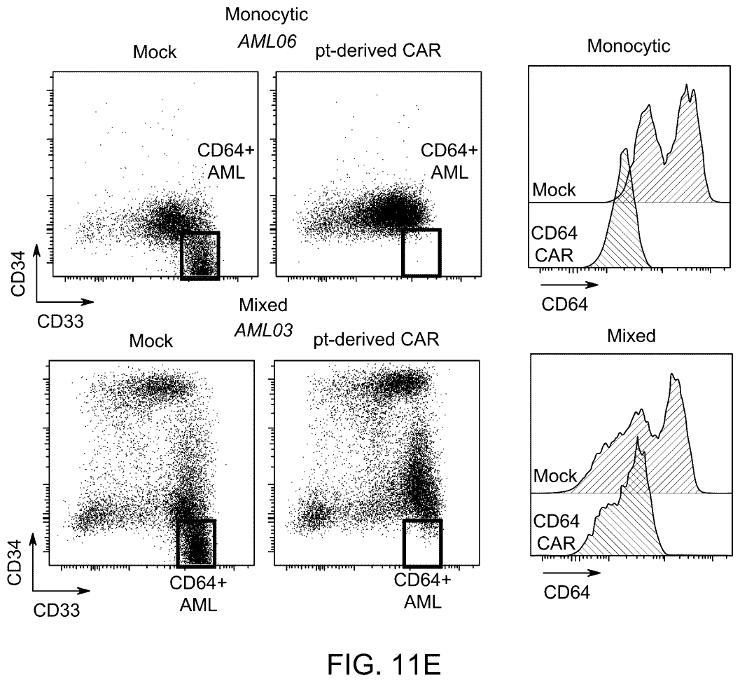

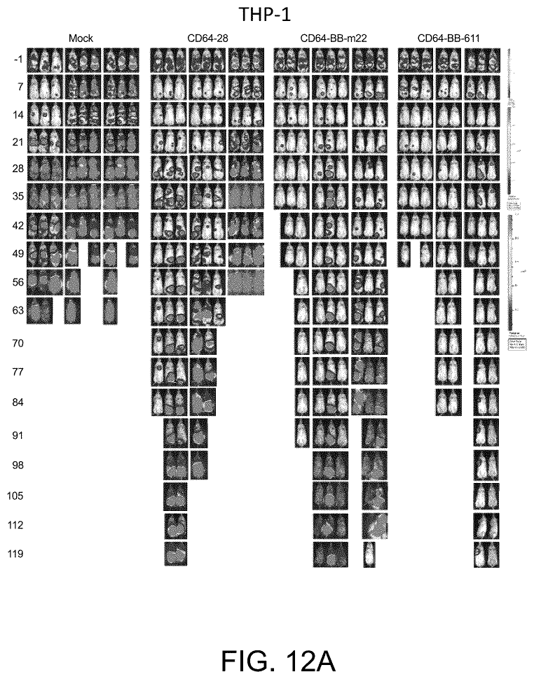

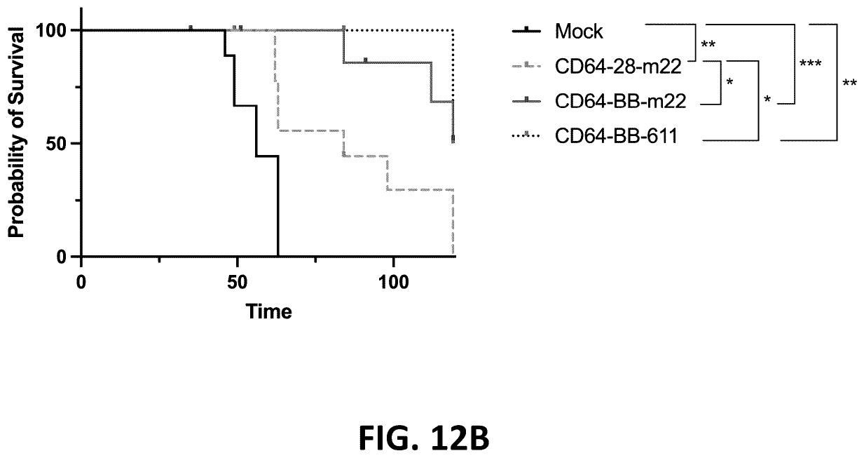

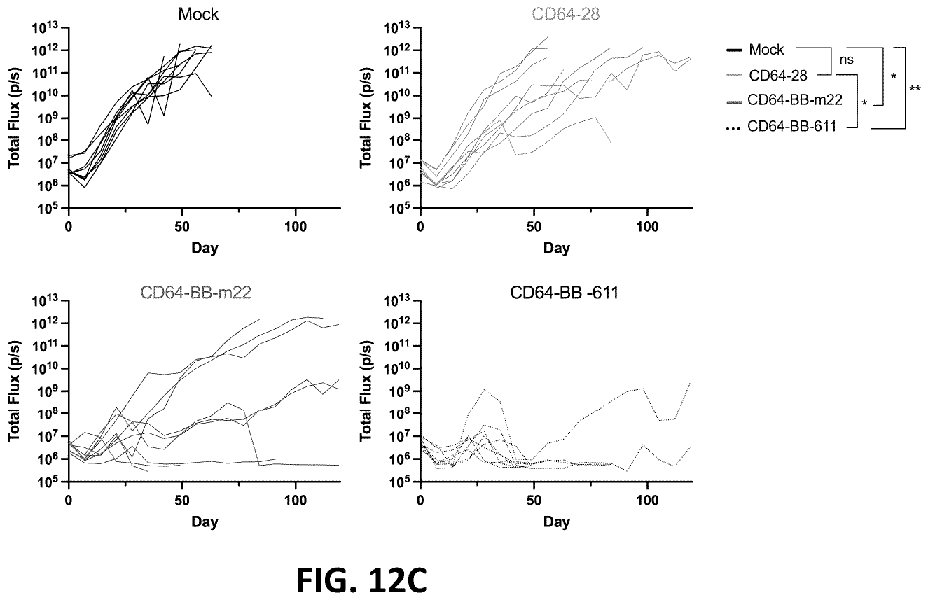

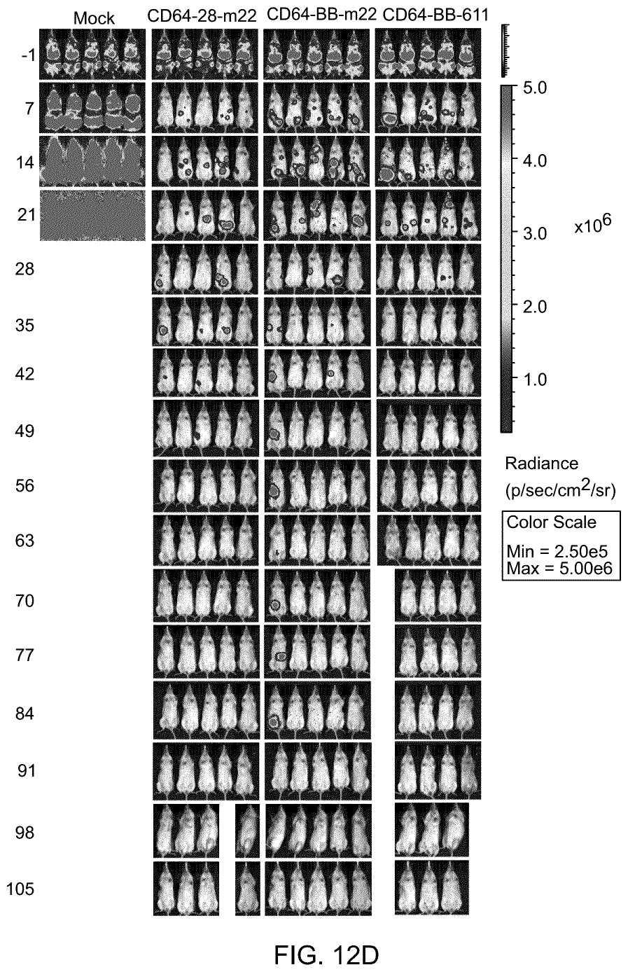

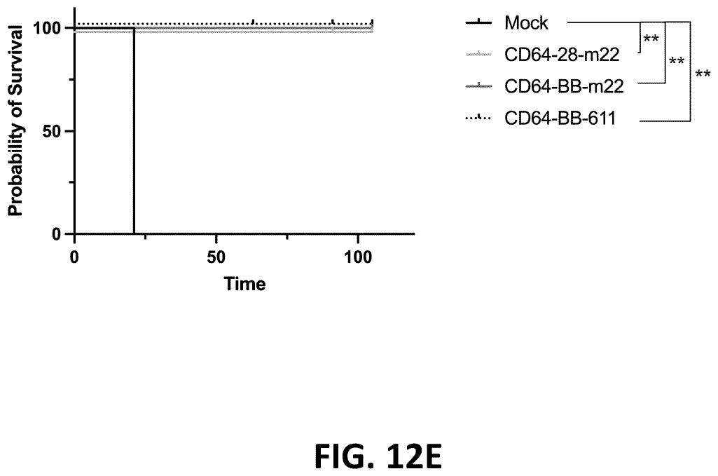

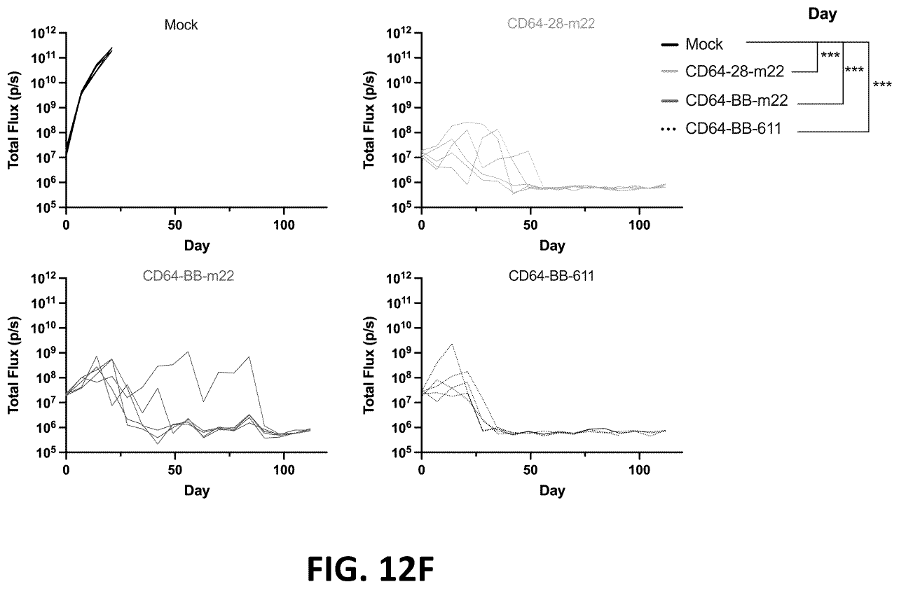

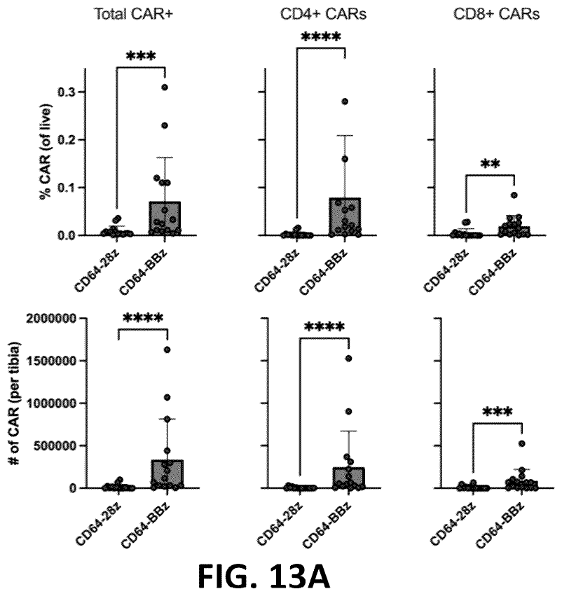

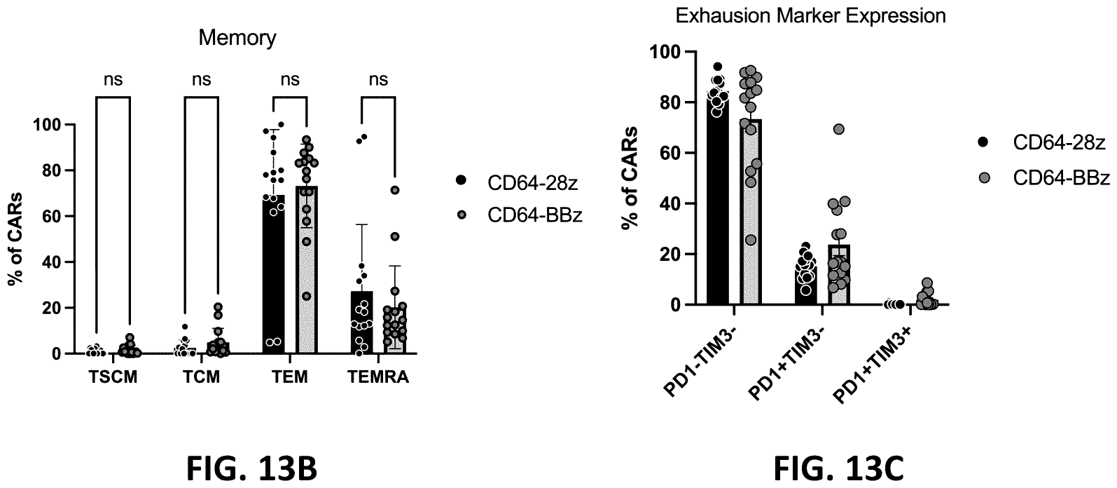

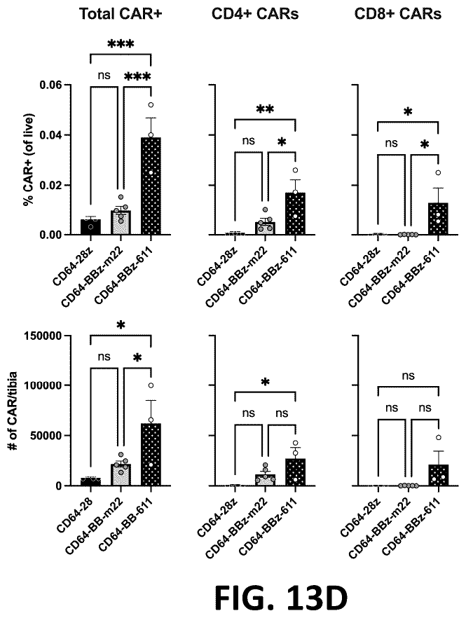

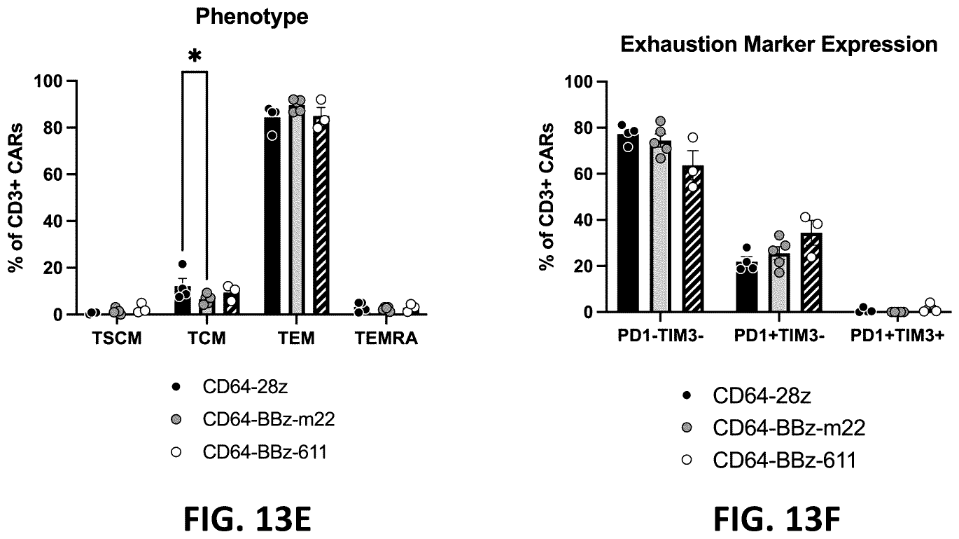

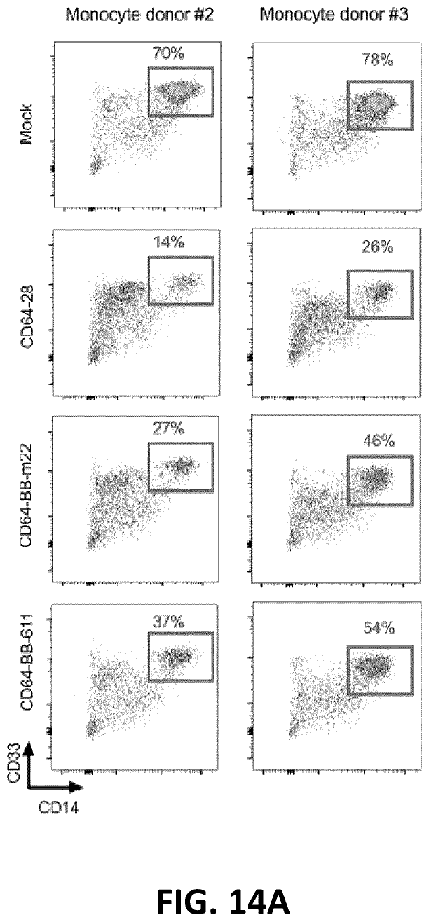

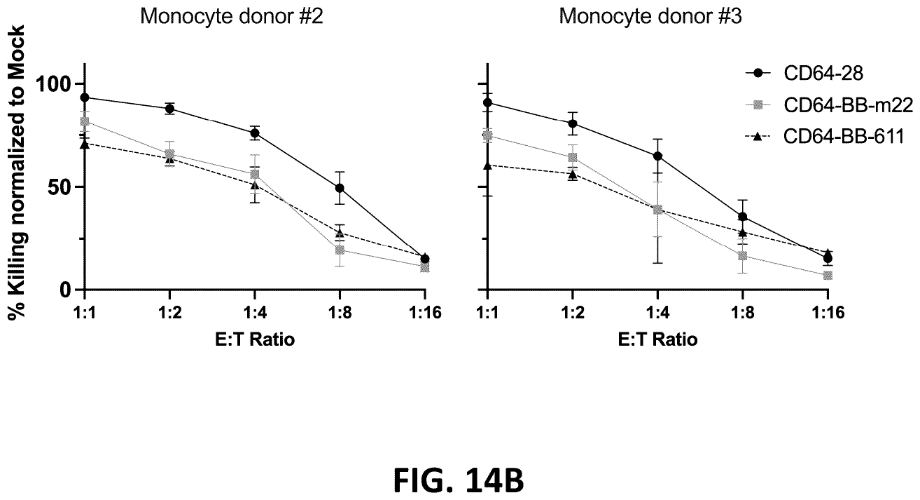

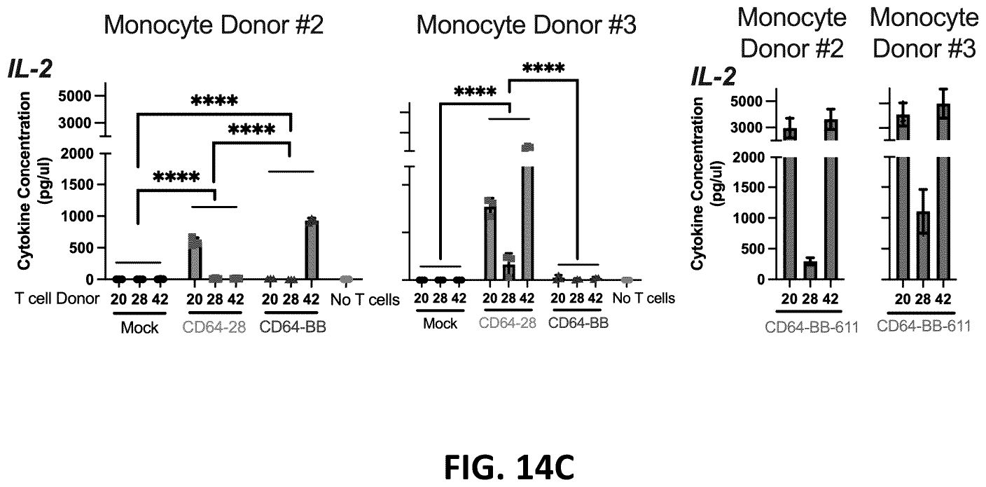

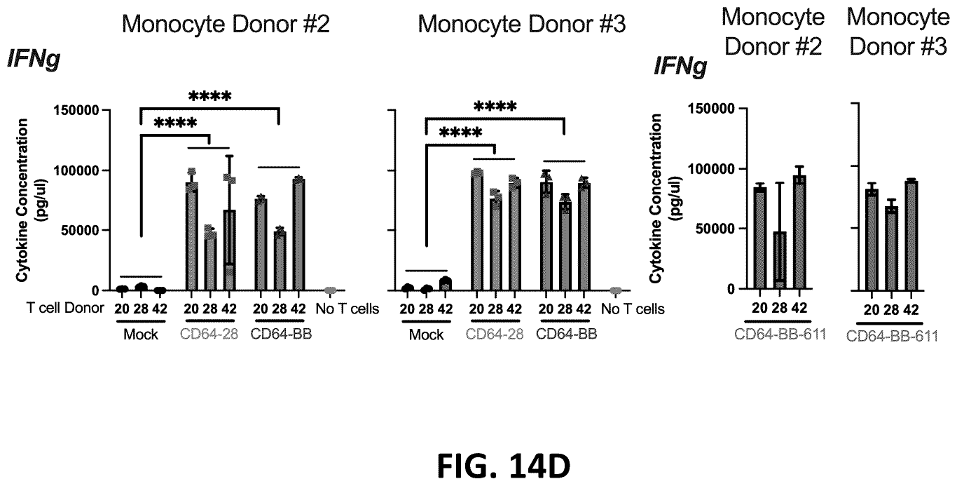

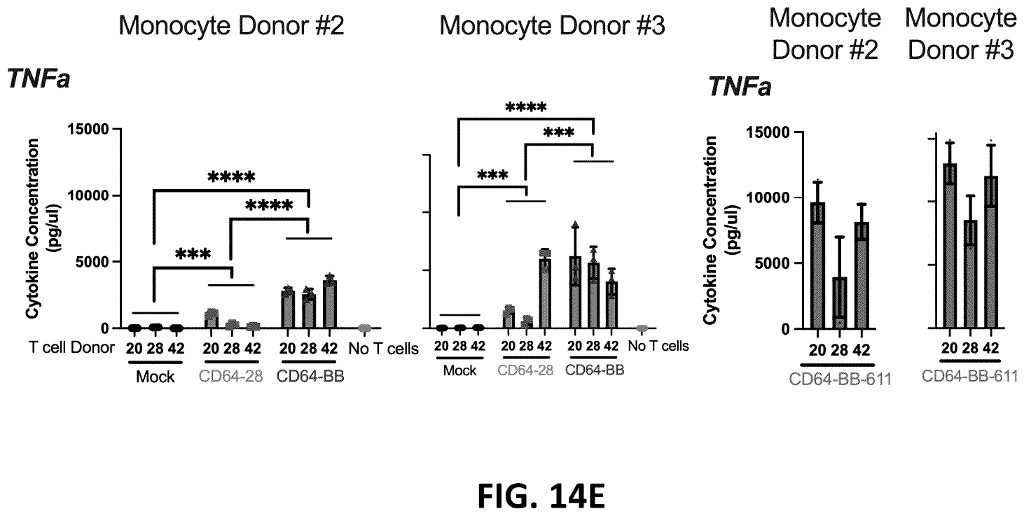

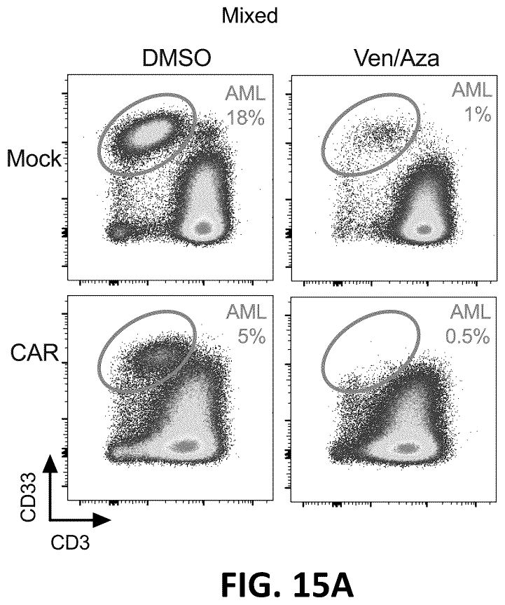

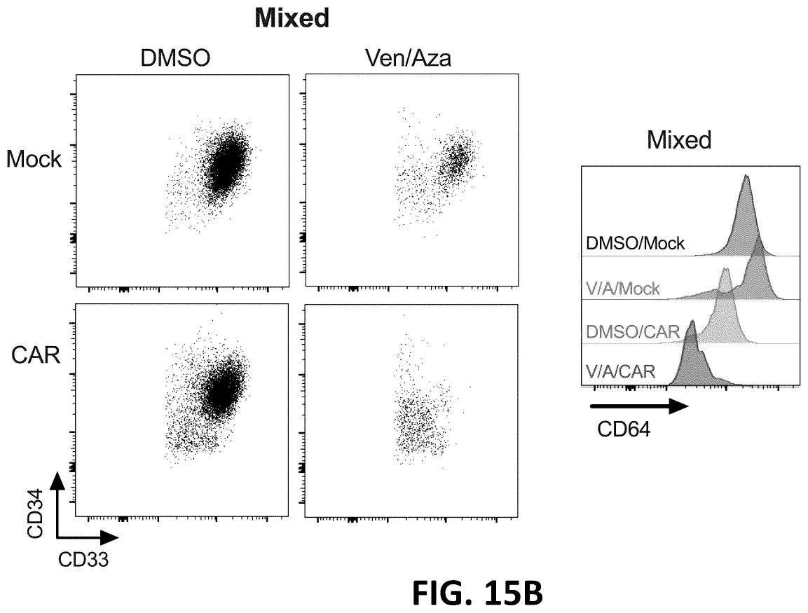

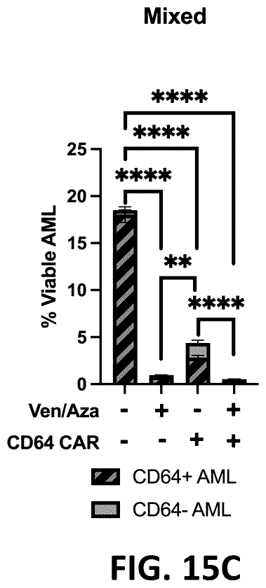

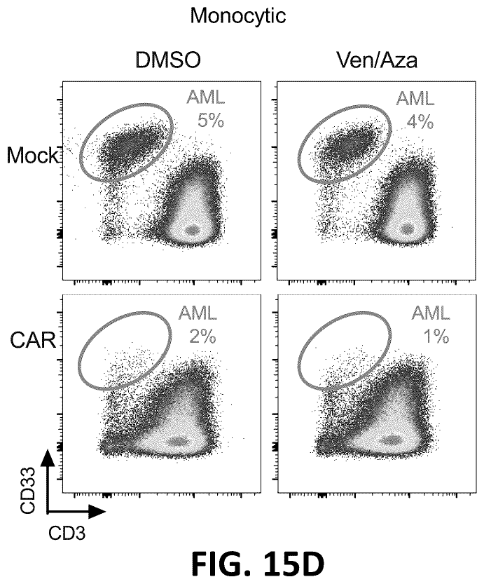

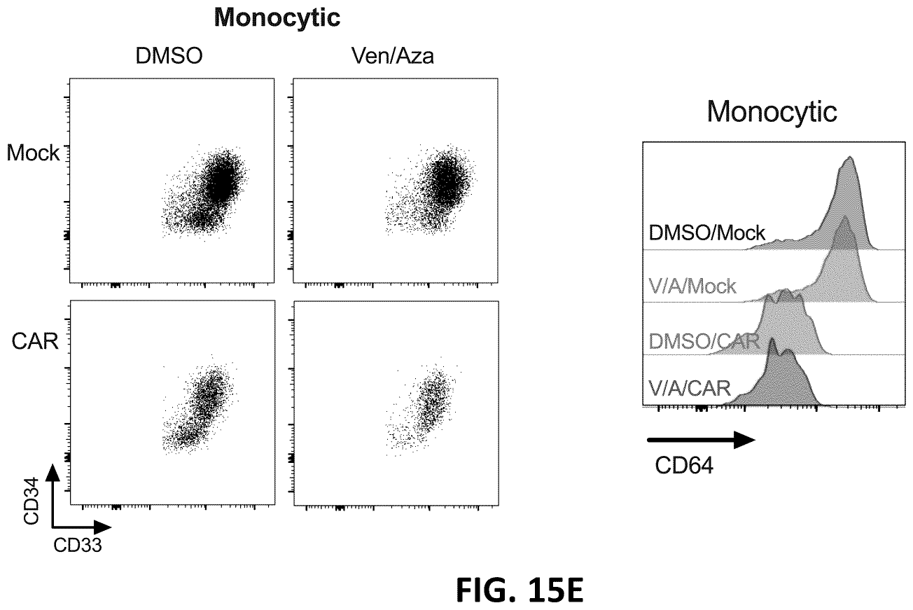

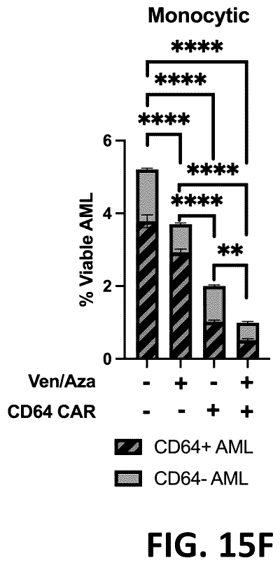

Efficacious CAR molecules are anticipated to elicit a cytotoxic response from CAR T cells upon engaging with antigen positive cells. To test the ability of the CD64 CAR constructs to elicit this effector function, CD64 CAR T cells were co-cultured with 1×10⁵ cells from MOLM14 (CD64low), THP-1 (CD64high), and K562 (CD64 negative) AML cell lines. Cells were co-cultured overnight at an E:T ratio of 1:1 or 1:4. The following day, viable AML cells were quantified by flow cytometry. Untransduced T cells from the same T cell donor (Mock T cells) were used as a negative control. Reductions in the viable AML cell populations were observed when CD64+ AML cells were cultured with CD64-28-m22, CD64-BB-m22, and CD64-BB-611 CAR T cells (FIG. 9A). Killing of target cells from three replicates was quantified (FIG. 9B).

To further characterize the cytotoxicity of CD64 CAR T cells, a luciferase-based killing assay was used to test each of the CD64 CAR construct's ability to kill MOLM14, THP-1, and K562 cells. Cells were co-cultured with CD64-28-m22, CD64-BB-m22, or CD64-BB-611 CAR T cells from three independent donors overnight at a range of E:T ratios (FIG. 9C). CD64-28-m22 CAR T cells demonstrated significantly higher cytotoxicity against THP-1 cells across all donors and higher cytotoxicity against MOLM14 cells in two of three donors. Both CD64-BB-m22 and CD64-BB-611 showed effective killing against both THP-1 and MOLM14 cells (FIG. 9C), consistent with previous results (FIGS. 9A-9B). Importantly, no significant killing of CD64-negative K562 cells by any CD64 CAR constructs was observed, supporting the antigen-dependence and specificity of these CARs.

To test the CD64 CAR T cells against primary AML, CD64 CAR T cells were co-cultured with 1×10⁵ patient-derived AML cells. Cells were co-cultured for 24 hours at various E:T ratios, and residual live AML cells were evaluated by flow cytometry. Patient-derived AML cells cultured with mock T cells showed a persistent CD64+ population, whereas each of the CD64 CAR T cell products reduced the CD64+ AML cells in a dose-dependent manner (FIGS. 9D-9E). CD64-28-m22, CD64-BB-m22, and CD64-BB-611 CAR T cells from multiple donors similarly eliminated CD64+ AML cells and showed killing of both monocytic and mixed monocytic/primitive AML patient cells (FIGS. 9D-9E). Collectively, these data demonstrate the cytotoxic function of CD64 CAR T cells against CD64-expressing AML cells.

### Secretion of T-Cell Effector Cytokines

Upon antigen stimulation, CAR T cells initiate effector functions such as the secretion of cytokines and the release of cytotoxic granules targeting antigen positive cells. To test the ability of CD64 CAR molecules to elicit cytokine production from CAR transduced T cells, 1×10⁵ CD64 CAR T cells (CD64-28-m22, CD64-BB-m22, or CD64-BB-611) were co-cultured with 1×10⁵ cells from CD64 expressing cell lines (MOLM14 and THP-1) or 1×10⁵ CD64+ cells from patient samples (monocytic and mixed monocytic/primitive primary AML). Cells were co-cultured for 24 hours and T cell effector cytokine (IL-2, IFNγ, and TNFα) production was measured by LegendPlex® assay (Biolegend) (FIGS. 10A-10F). Higher production of the canonical T cell effector cytokines IL-2, IFNγ, and TNFα was observed after stimulation with THP-1 cells (CD64high) relative to MOLM14 (CD64low) (FIGS. 10A-10C). No cytokine production was observed from mock T cells (untransduced T cells) co-cultured with THP-1 or MOLM14 cells. These results demonstrate that AML cell lines do not produce these cytokines, and transduction with a CD64 CAR was required for T cell release of these cytokines (FIGS. 10A-10C). Furthermore, CD64 CAR T cells did not demonstrate antigen-independent cytokine production as there was no cytokine production upon co-culture with the CD64-negative cell line, K562 (FIGS. 10A-10C). Despite the expected donor-to-donor variation in cytokine production that is common in CAR T cell products, increased IL-2 secretion by CD64-28-m22 CAR T cells was observed (FIG. 10A). A high level of IL-2 secretion by CD64-BB-611 CAR T cells was also observed in co-culture with CD64+ cell lines (FIG. 10A). Conversely, increased TNFα secretion from CD64-BB-m22 CAR T cells was observed (FIG. 10C). A high level of TNFα secretion by CD64-BB-611 CAR-T cells was also observed in co-culture with CD64+ cell lines (FIG. 10C). It was found that IFNγ was secreted in similar amounts from both of CD64-28-m22 and CD64-BB-m22 CAR T cell products in response to THP-1 cells and at a slightly higher level by CD64-BB-m22 CAR T cells in response to MOLM14 (FIG. 10B). A similarly high level of IFNγ secretion by CD64-BB-611 CAR-T cells was also observed in co-culture with CD64+ cell lines (FIG. 10B).

Upon co-culture with patient samples, CD64-28-m22 CAR T cells demonstrated the ability to produce significant amounts of IL-2, INFγ, and TNFα in response to both monocytic and mixed primitive/monocytic patient-derived AML samples (FIGS. 10D-10F). Similarly, CD64-BB-m22 CAR T cells secreted significant amounts of IFNγ and TNFα in response to both monocytic and mixed primitive/monocytic patient-derived AML cells (FIGS. 10E-10F). CD64-BB-611 CAR-T cells secreted particularly high levels of IL-2 and TNFα as well as comparably high levels of INFγ (FIGS. 10D-10F). All cytokine data was analyzed in triplicate using three independent T cell donors. Statistical analysis was conducted using two-way ANOVA with Turkey's multiple comparison test. Collectively, these data demonstrated that both CD64-28-m22, CD64-BB-m22, and CD64-BB-611 CARs elicited antigen-dependent cytokine production by transduced T cells in response to AML cells, supporting their effector function against leukemia expressing the target antigen.

### Manufacturing of CD64 CAR T Cells

A concern with CAR T therapy for AML has been the fitness or ability to manufacture CAR T cells from patient-derived T cells. To address this concern, T cells from an AML patient sample biobank were transduced with CD64-BB-m22 CAR in parallel to healthy donor T cells. Patient-derived T cells proliferated similarly to healthy donor T cells during CAR T manufacturing (FIG. 11A). Patient derived T cells and healthy donor T cells were transduced with CAR with comparable efficiency in one donor but reduced efficiency in another (FIG. 11B). No significant difference in T cell proliferation was noted between patient-derived CD64-BB-m22 CAR T cells and healthy donor CD64-BB-m22 CAR T cells in co-culture with MOLM14 target leukemia cells (FIG. 11C).

In a luciferase killing assay, it was shown that patient derived CD64-BB-m22 CAR T cells killed MOLM14 cells in a dose dependent manner, similar to the healthy donor-derived CD64-BB-m22 CAR T cells shown in FIG. 9C (FIG. 11D). Patient-derived CD64-BB-m22 CAR T cells were next cultured with their respective monocytic and mixed AML patient samples overnight. CD64-BB-m22 CAR T cells eliminated CD64+ AML cells in these patient samples (FIG. 11E). This data suggest that CD64 CAR T cells can be manufactured from patient derived T cells and are effective against CD64 expressing AML.

### CAR T Cells in CD64 Leukemia Xenograft Models

In vivo efficacy of CD64 CAR T cells was also assessed in mice engrafted with THP-1 (CD64high) leukemia cells, which produces a more resistant disease than MOLM14 in xenograft models. Mice were inoculated with 1×10⁶ luciferase-positive THP1 cells by tail vein injection on day −4. Engraftment was confirmed by luciferase-based bioluminescent in vivo imaging on a Xenogen IVIS® platform on day 0. Mice were infused with 5×10⁶ CD64 CAR+ T cells or an equivalent number of mock T cells on day 0 and monitored by BLI weekly as described above in FIG. 6 (FIG. 12A). The experiment was replicated three times using CAR T cells manufactured from three independent human T cell donors. Mice treated with mock T cells all succumbed to leukemia progression by day 70 and none died of the expected complication of xenogeneic graft-versus-host disease (xGVHD) which leads to mice being euthanized due to predefined humane endpoints in the absence of detectable leukemia. This is a well-described complication of infusing human T cells into immunodeficient mice and was not related to the function of the CAR or the CAR T cells. CD64-BB-611 CAR T cells resulted in complete leukemia clearance in all 8 mice 14 days after treatment. None of these mice succumbed to leukemia and all deaths were due to xGVHD. Eight of the nine mice treated with CD64-28-m22 and three of nine treated with CD64-BB-m22 CAR T cells developed progressive leukemia. This data demonstrates that each of the CD64 CAR constructs has in vivo efficacy against established AML in xenograft models. Furthermore, the data demonstrates superior survival and leukemic clearance by CD64 CAR T cells that utilize a 4-1BB costimulatory domain, with the best leukemia control by CD64-BB-611 CAR T cells (FIG. 12B-12C).

To assess the in vivo efficacy of CD64-BB-611 CAR T cells, NSG mice were engrafted with 1×10⁶ luciferase-expressing MOLM14 cells on day −4. Leukemia engraftment was confirmed by luciferase-based BLI on day −1. On day 0, mice were treated with 3×10⁶ CD64 CAR+ T cells or an equivalent dose of mock T cells. Mice were subsequently imaged by BLI weekly to track leukemia burden (FIG. 12D). The experiment was replicated a third time as shown in FIG. 6A using T cells generated from a third healthy donor to include a comparison of CD64-BB-611 CAR T cells. By day 30, all 5 mice treated with untransduced control T cells had expired with leukemia. By day 91, all mice treated with CD64-28-m22, CD64-BB-m22, and CD64-BB-611 CAR T cells achieved a leukemia free state. All 5 mice treated with CD64-BB-611 had a complete clearance of leukemia as early as 35 days after CAR T administration, and no mice in this group died from leukemia progression. Leukemia free survival was 100% for all CD64 CAR-T cell treated mice (FIG. 12E). Quantification of total bioluminescent flux per mouse demonstrated decreased leukemia burden in mice treated with CD64-28-m22, CD64-BB-m22 and CD64-BB-611 CAR-T cells as compared to mock treated mice (FIG. 12F).

### CD64 CAR T Cell Persistence in Xenograft Models

As persistence of CAR T cells has been shown to impact outcomes in lymphoid leukemias, the persistence of CD64 CAR T cells after clearance of MOLM14 leukemia was evaluated. NSG mice were engrafted with 1×10⁶ MOLM14 cells at day −4. Mice were then treated with 5×10⁶ CD64-28-m22 or CD64-BB-m22 CAR+ T cells. At day 35, mice were euthanized and bone marrow was evaluated by flow cytometry to assess persistent CAR T cells. There was a significantly higher percentage and absolute number of CD64-BB-m22 CAR T cells in the marrow (mean 335,811 cells/tibia) after leukemia clearance as compared to CD64-28-m22 CAR T cells (15,148 cells/tibia; p<0.0001 by Mann-Whitney) (FIG. 13A). A similar pattern was observed in the CD4+ and CD8+ CAR T cell compartments (FIG. 13A). Phenotypically, the majority of persisting CAR T cells were T effector memory cells (TEM) with no difference observed between the CAR co-stimulatory domains (69% of CD64-28-m22 and 73% of CD64-BB-m22; p=0.95) (FIG. 13B). The remaining cells predominantly had a T effector memory with re-expression of CD45RA (TEMRA) phenotype (FIG. 13B). The majority of persisting CAR T cells did not express exhaustion markers (FIG. 13C). This data suggests that CD64 CAR T cells with 4-1BB co-stimulatory domains display an improved in vivo efficacy against monocytic AML and prolonged persistence of CAR T cells. Furthermore, CD64-BB-611 CAR T cells that incorporate a humanized binder showed the most in vivo efficacy against the monocytic THP-1 cell line.

The previous experiment was repeated to include the CD64-BB-611 CAR and to investigate CAR T cell persistence and phenotype at a later time point after leukemia clearance. Mice cleared MOLM14 leukemia and were euthanized after 105 days. An average of 62,113 CD64-BB-611 CAR+ cells were detected per femur as compared to 7,992 CD64-28-m22 CAR+ cell per femur and 21,655 CD64-BB-m22 CAR+ cells per femur, suggesting further improvement in persistence with the CD64-BB-611 CAR construct over CD64-BB-m22 (FIG. 13D). CD64-BB-611 also produced TEM cells, and the majority of CD64 CAR T cells remained negative for exhaustion markers PD1 and TIM3 at this later time point (FIGS. 13E-13F).

### CD64 CAR T Cell Toxicity Against Monocytes

Given the known expression pattern of CD64 on monocytes and macrophages, CD64 CAR T cell treatment may result in depletion of these cell populations as an on-target/off-tumor toxicity. To test this, CD64 CAR-T cells from three different T cell donors were co-cultured with normal monocytes from two different, healthy blood donors. All CD64 CAR-T cells reduced viable populations of classic and non-classic CD14+ monocytes. CD64-28-m22 CAR T cells generated the greatest monocyte killing after 24 hours at a 1:2 E:T ratio (FIGS. 14A-14B). CD64 CAR-T cells produced IL-2, IFNγ, and TNFα effector cytokines after 24 hours of co-culture with normal monocytes (FIG. 14C) and showed a similar pattern of cytokine expression as was seen after co-culture with AML cells (FIGS. 10A-10F). Statistical analysis was performed using two-way ANOVA and Turkeys multiple comparison test. Thus, reduction of normal monocytes is an anticipated on-target/off-tumor toxicity of CD64 CAR T cell treatment. This may have further therapeutic potential to be harnessed in applications to deplete macrophages in the tumor microenvironment of other malignancies. Tumor associated macrophages (TAMs) have been shown to negatively impact cancer progression and prognosis by infiltrating the tumor microenvironment and driving a cytokine and growth facture milieu which promotes cancer cell division and metastasis, accelerates angiogenesis, supports maintenance of cancer stem cells, and produces an immunosuppressive effect through cytokine secretion and recruitment of suppressive regulatory T cells. (Li et al., J. Immunother. Cancer., 9(1):e001341 (2021) doi: 10.1136/jitc-2020-001341. PMID: 33504575; DeNardo et al., Nat. Rev. Immunol., 19(6):369-382 (2019). doi: 10.1038/s41577-019-0127-6. PMID: 30718830; PMCID: PMC7339861). Methods of decreasing TAMs in the tumor microenvironment is an active area of study in clinical trials for a multitude of hematologic and solid tumors including AML, multiple myeloma, lymphomas, ovarian cancer, lung cancer, pancreatic cancer, sarcomas, colorectal cancer and in other malignancies including advanced/metastatic cancer of any origin. There is potential in harnessing the noted anti-monocyte/macrophage, on-target, off-tumor effect of CD64 CAR-T cell therapy to deplete these TAMs in various tumor types, in addition to the intended use in venetoclax relapsed/refractory AML, which is the primary subject of this discussion.

### CD64 CAR T Cell and Ven/Aza Combination Therapy

The effect of combining CD64 CAR-T cell therapy in series with ven/aza treatment on monocytic and mixed monocytic/primitive primary patient AML was assessed. Venetoclax with a hypomethylating agent, often azacitadine, is frequently used in standard of care AML therapy. Unfortunately, patients with monocytic AML are more likely to relapse after, or be initially refractory to, this combination. Monocytic or mixed AML patient samples (1×10⁵ cells per replicate) were thawed and cultured in vitro in IMDM media supplemented with BIT, FBS, BME, and LDL with supportive myeloid cytokines, FLT3, IL-3, and SCF at 10 ng/mL. Cells were treated for 48 hours with either 500 nM venetoclax in DMSO and 1.5 μM azacitadine in PBS or 500 nM DMSO alone as a control. 50,000 CD64-BB-m22 CAR-T cells or mock transduced T cells were then added, and culture was continued for an additional 48 hours. Following the 4 days of culture, viable AML cell number and CD64 expression was assessed by flow cytometry. Mixed monocytic/primitive AML was sensitive to ven/aza alone (21-fold reduction in viable AML cells compared to control, p<0.0001), but cells remaining at the end of treatment were highly CD64+(FIGS. 15A-15C), in line with results described previously (Pei, et. al., Cancer Discovery (2020)). Treatment with CD64-BB-m22 CAR-T cells alone decreased the number of viable AML cells (4.2-fold decrease in all AML) and nearly eliminated CD64+ AML. In sequence treatment with both ven/aza and CD64-BB-m22 CAR-T decreased viable AML by 34.25-fold and CD64+ AML by 353.5-fold (p<0.0001). Treating the monocytic AML patient sample with ven/aza did not significantly change AML cell viability (p=0.998) (FIG. 16D) or impactfully alter CD64 expression (FIGS. 15E-15F). Treating monocytic AML with CD64 CAR-T with or without ven/aza significantly decreased CD64+ AML by >40-fold (p<0.0001) (FIG. 15F). This data supports the efficacy of CD64 CAR-T therapy in primary monocytic AML and demonstrates the ability of ven/aza and CD64 CAR-T combination therapy to effectively target ven/aza resistant AML.

### Example 7—Exemplary CD64-Binding Molecules

This Example provides the amino acid sequences of exemplary scFvs having the ability to bind CD64. The [symbol] , linker, and framework sequences of each also are provided and delineated.

### Anti-CD64 Clone #1

Full-Length scFV

(SEQ ID NO: 1)

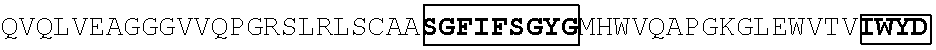

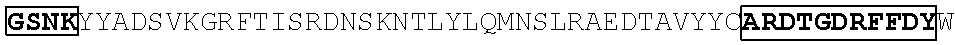

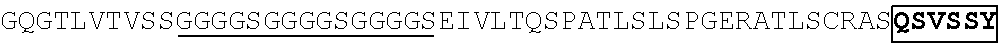

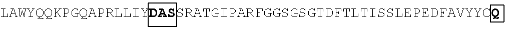

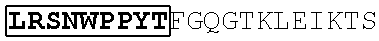

### VH Domain:

(SEQ ID NO: 31)

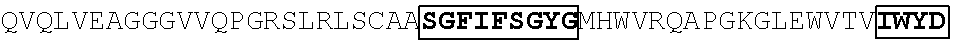

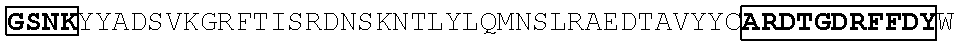

GQGTLVTVSS

### Framework Region 1 of VH Domain:

(SEQ ID NO: 27)
QVQLVEAGGGVVQPGRSLRLSCAA

### CDR1 of VH Domain:

(SEQ ID NO: 24)
SGFIFSGYG

### Framework Region 2 of VH Domain:

(SEQ ID NO: 28)
MHWVRQAPGKGLEWVTV

### CDR2 of VH Domain:

(SEQ ID NO: 25)
IWYDGSNK

### Framework Region 3 of VH Domain:

(SEQ ID NO: 29)
YYADSVKGRFTISRDNSKNTLYLQMNSLRAEDTAVYYC

### CDR3 of VH Domain:

(SEQ ID NO: 26)
ARDTGDRFFDY

### Framework Region 4 of VH Domain:

(SEQ ID NO: 30)
WGQGTTVTVSS

In some cases, Framework Region 4 of VH domain can be WGQGTLVTV (SEQ ID NO:381) and/or an “SS” sequence can be added to the 5′ end of a linker set forth in SEQ ID NO: 420.

### Linker:

(SEQ ID NO: 420)
GGGGSGGGGSGGGGS

### VL Domain:

(SEQ ID NO: 39)

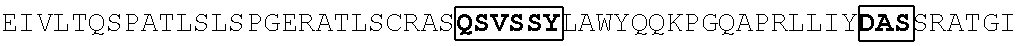

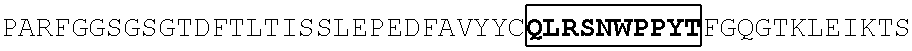

### Framework Region 1 of VL Domain:

(SEQ ID NO: 35)
EIVLTQSPATLSLSPGERATLSCRAS

### CDR1 of VL Domain:

(SEQ ID NO: 32)
QSVSSY

### Framework Region 2 of VL Domain:

(SEQ ID NO: 36)
LAWYQQKPGQAPRLLIY

### CDR2 of VL Domain:

(SEQ ID NO: 33)
DAS

### Framework Region 3 of VL Domain:

(SEQ ID NO: 37)
SRATGIPARFGGSGSGTDFTLTISSLEPEDFAVYYC

### CDR3 of VL Domain:

(SEQ ID NO: 34)
QLRSNWPPYT

### Framework Region 4 of VL Domain:

(SEQ ID NO: 38)
FGQGTKLEIKTS

In some cases, Framework Region 4 of VL domain can lack the 3′ “TS” sequence: FGQGTKLEIK (SEQ ID NO:382)

### Anti-CD64 Clone #2

Full-Length scFV

(SEQ ID NO: 1)

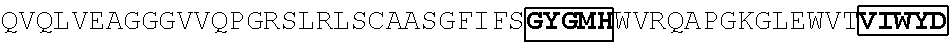

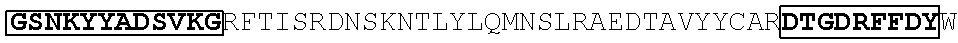

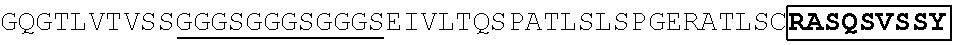

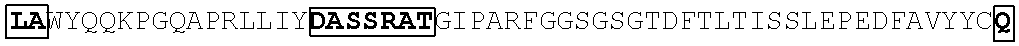

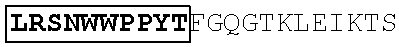

### VH Domain:

(SEQ ID NO: 47)

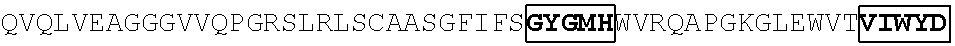

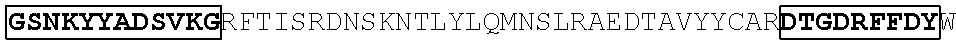

GQGTLVTVSS

### Framework Region 1 of VH Domain:

(SEQ ID NO: 43)
QVQLVEAGGGVVQPGRSLRLSCAASGFIFS

### CDR1 of VH Domain:

(SEQ ID NO: 40)
GYGMH

### Framework Region 2 of VH Domain:

(SEQ ID NO: 44)
WVRQAPGKGLEWVT

### CDR2 of VH Domain:

(SEQ ID NO: 41)
VIWYDGSNKYYADSVKG

### Framework Region 3 of VH Domain:

(SEQ ID NO: 45)
FTISRDNSKNTLYLQMNSLRAEDTAVYYCAR

### CDR3 of VH Domain:

(SEQ ID NO: 42)
DTGDRFFDY

### Framework Region 4 of VH Domain:

(SEQ ID NO: 46)
WGQGTLVTVSS

In some cases, Framework Region 4 of VH domain can be WGQGTLVTV (SEQ ID NO:383) and/or an “SS” sequence can be added to the 5′ end of a linker set forth in SEQ ID NO: 420.

### Linker:

(SEQ ID NO: 420)
GGGGSGGGGSGGGGS

### VL Domain:

(SEQ ID NO: 55)

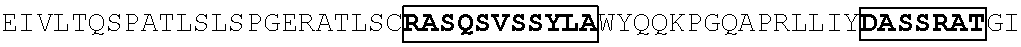

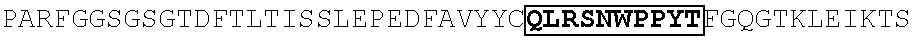

### Framework Region 1 of VL Domain:

(SEQ ID NO: 51)
EIVLTQSPATLSLSPGERATLSC

### CDR1 of VL Domain:

(SEQ ID NO: 48)
RASQSVSSYLA

### Framework Region 2 of VL Domain:

(SEQ ID NO: 52)
WYQQKPGQAPRLLIY

### CDR2 of VL Domain:

(SEQ ID NO: 49)
DASSRAT

### Framework Region 3 of VL Domain:

(SEQ ID NO: 53)
GIPARFGGSGSGTDFTLTISSLEPEDFAVYYC

### CDR3 of VL Domain:

(SEQ ID NO: 50)
QLRSNWPPYT

### Framework Region 4 of VL Domain:

(SEQ ID NO: 54)
FGQGTKLEIKTS

In some cases, Framework Region 4 of VL domain can lack the 3′ “TS” sequence: FGQGTKLEIK (SEQ ID NO:384)

(SEQ ID NO: 384)
FGQGTKLEIK

### Anti-CD64 Clone #3

Full scFV

(SEQ ID NO: 3)

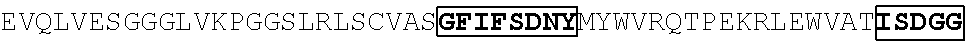

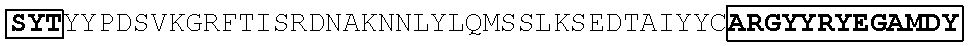

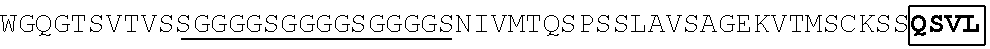

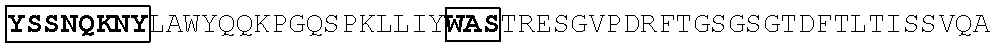

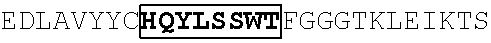

### VH Domain:

(SEQ ID NO: 385)

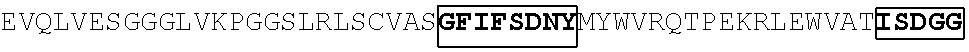

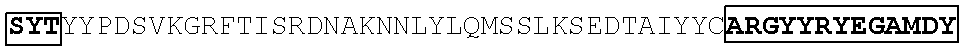

WGQGTSVTVSS

### Framework Region 1 of VH Domain:

(SEQ ID NO: 386)
EVQLVESGGGLVKPGGSLRLSCVAS

### CDR1 of VH Domain:

(SEQ ID NO: 387)
GFIFSDNY

### Framework Region 2 of VH Domain:

(SEQ ID NO: 388)
MYWVRQTPEKRLEWVAT

### CDR2 of VH Domain:

(SEQ ID NO: 389)
ISDGGSYT

### Framework Region 3 of VH Domain:

(SEQ ID NO: 390)
YYPDSVKGRFTISRDNAKNNLYLQMSSLKSEDTAIYYC

### CDR3 of VH Domain:

(SEQ ID NO: 391)
ARGYYRYEGAMDY

### Framework Region 4 of VH Domain:

(SEQ ID NO: 392)
WGQGTSVTVSS

In some cases, Framework Region 4 of VH domain can be WGQGTSVTV (SEQ ID NO:393) and/or an “SS” sequence can be added to the 5′ end of a linker set forth in SEQ ID NO: 420.

### Linker:

(SEQ ID NO: 420)
GGGGSGGGGSGGGGS

### VL Domain:

(SEQ ID NO: 394)

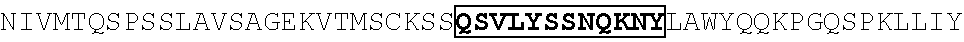

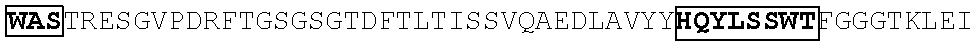

KTS

### Framework Region 1 of VL Domain:

(SEQ ID NO: 395)
NIVMTQSPSSLAVSAGEKVTMSCKSS

### CDR1 of VL Domain:

(SEQ ID NO: 396)
QSVLYSSNQKNY

### Framework Region 2 of VL Domain:

(SEQ ID NO: 397)
LAWYQQKPGQSPKLLIY

### CDR2 of VL Domain:

(SEQ ID NO: 398)
WAS

### Framework Region 3 of VL Domain:

(SEQ ID NO: 399)
TRESGVPDRFTGSGSGTDFTLTISSVQAEDLAVYYC

### CDR3 of VL Domain:

(SEQ ID NO: 400)
HQYLSSWT

### Framework Region 4 of VL Domain:

(SEQ ID NO: 401)
FGGGTKLEIKTS

In some cases, Framework Region 4 of VL domain can lack the 3′ “TS” sequence:

(SEQ ID NO: 402)
FGGGTKLEIK

### Anti-CD64 Clone #4

Full scFV

(SEQ ID NO: 3)

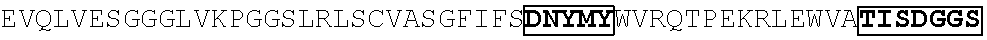

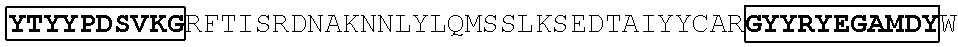

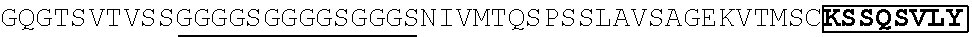

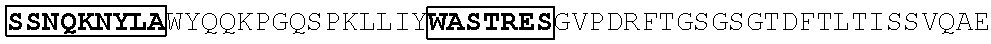

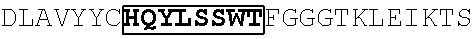

### VH Domain:

(SEQ ID NO: 403)

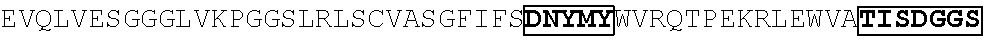

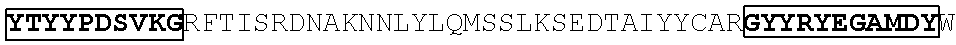

GQGTSVTVSS

### Framework Region 1 of VH Domain:

(SEQ ID NO: 404)
EVQLVESGGGLVKPGGSLRLSCVASGFIFS

### CDR1 of VH Domain:

(SEQ ID NO: 405)
DNYMY

### Framework Region 2 of VH Domain:

(SEQ ID NO: 406)
WVRQTPEKRLEWVA

### CDR2 of VH Domain:

(SEQ ID NO: 407)
TISDGGSYTYYPDSVKG

### Framework Region 3 of VH Domain:

(SEQ ID NO: 408)
RFTISRDNAKNNLYLQMSSLKSEDTAIYYCAR

### CDR3 of VH Domain:

(SEQ ID NO: 409)
GYYRYEGAMDY

### Framework Region 4 of VH Domain:

(SEQ ID NO: 410)
WGQGTSVTVSS

In some cases, Framework Region 4 of VH domain can be WGQGTSVTV (SEQ ID NO:411) and/or an “SS” sequence can be added to the 5′ end of a linker set forth in SEQ ID NO: 420.

### Linker:

(SEQ ID NO: 420)
GGGGSGGGGSGGGGS

### VL Domain:

(SEQ ID NO: 412)

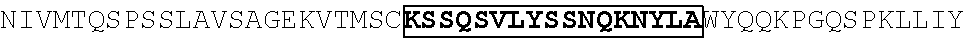

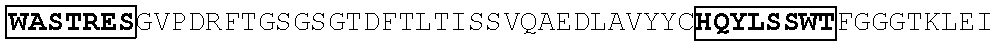

K

### Framework Region 1 of VL Domain:

(SEQ ID NO: 413)
NIVMTQSPSSLAVSAGEKVTMSC

### CDR1 of VL Domain:

(SEQ ID NO: 421)
KSSQSVLYSSNQKNYLA

### Framework Region 2 of VL Domain:

(SEQ ID NO: 414)
WYQQKPGQSPKLLIY

### CDR2 of VL Domain:

(SEQ ID NO: 415)
WASTRES

### Framework Region 3 of VL Domain:

(SEQ ID NO: 416)
GVPDRFTGSGSGTDFTLTISSVQAEDLAVYYC

### CDR3 of VL Domain:

(SEQ ID NO: 417)
HQYLSSWT

### Framework Region 4 of VL Domain:

(SEQ ID NO: 418)
FGGGTKLEIKTS

In some cases, Framework Region 4 of VL domain can lack the 3′ “TS” sequence:

(SEQ ID NO: 419)
FGGGTKLEIK

### Anti-CD64 Clone #5

Full scFV

(SEQ ID NO: 2)
QVQLVESGGGWQPGRSLRLSCSSSGFIFSDNYMYWVRQAPGKGLEWVATI
SDGGSYTYYPDSVKGRFTISRDNSKNTLFLQMDSLRPEDTGVYFCARGYY
RYEGAMDYWGQGTPVTVSSGGGGSGGGGSGGGGSDIQLTQSPSSLSASVG
DRVTITCKSSQSVLYSSNQKNYLAWYQQKPGKAPKLLIYWASTRESGVPS
RFSGSGSGTDFTFTISSLQPEDIATYYCHQYLSSWTFGQGTKLEIKTS

What is claimed is:

1. A polypeptide comprising:
a) a first domain comprising SEQ ID NO: 1;
b) a second domain comprising an amino acid sequence of SEQ ID NO: 4;
c) a third domain comprising an amino acid sequence of SEQ ID NO: 5;
d) a fourth domain comprising an amino acid sequence of SEQ ID NO: 6; and
e) a fifth domain comprising an amino acid sequence of SEQ ID NO: 7.

2. The polypeptide of claim 1, wherein said first domain further comprises an amino acid sequence of SEQ ID NO: 23.

3. The polypeptide of claim 1, wherein said polypeptide comprises an amino acid sequence of SEQ ID NO: 11.

4. A nucleic acid molecule comprising a nucleic acid sequence encoding a polypeptide, wherein said polypeptide comprises:
a) a first domain comprising SEQ ID NO: 1;
b) a second domain comprising an amino acid sequence of SEQ ID NO: 4;
c) a third domain comprising an amino acid sequence of SEQ ID NO: 5;
d) a fourth domain comprising an amino acid sequence of SEQ ID NO: 6; and
e) a fifth domain comprising an amino acid sequence of SEQ ID NO: 7.

5. The nucleic acid molecule of claim 4, wherein said first domain further comprises an amino acid sequence of SEQ ID NO: 23.

6. The nucleic acid molecule of claim 4, wherein said polypeptide comprises an amino acid sequence of SEQ ID NO: 11.

7. The nucleic acid molecule of claim 4, wherein said nucleic acid molecule comprises a nucleic acid sequence of SEQ ID NO: 17.

8. A population of cells comprising nucleic acid encoding a polypeptide, wherein said polypeptide comprises:
a) a first domain comprising SEQ ID NO: 1;
b) a second domain comprising an amino acid sequence of SEQ ID NO: 4;
c) a third domain comprising an amino acid sequence of SEQ ID NO: 5;
d) a fourth domain comprising an amino acid sequence of SEQ ID NO: 6; and
e) a fifth domain comprising an amino acid sequence of SEQ ID NO: 7.

9. The population of cells of claim 8, wherein said first domain further comprises an amino acid sequence of SEQ ID NO: 23.

10. The population of cells of claim 8, wherein said polypeptide comprises an amino acid sequence of SEQ ID NO: 11.

11. The population of cells of claim 8, wherein said nucleic acid comprises a nucleic acid sequence of SEQ ID NO: 17.

12. The population of cells of claim 8, wherein said population comprises immune cells.

13. The population of cells of claim 12, wherein said immune cells are T-cells or Natural Killer (NK) cells.

14. The population of cells of claim 8, wherein said population comprises T-cells.

15. The population of cells of claim 8, wherein said population comprises NK cells.

16. A method of treating acute myeloid leukemia (AML) in a mammal, wherein said method comprises administering, to said mammal, a population of cells comprising nucleic acid encoding a polypeptide, wherein said polypeptide comprises:
a) a first domain comprising SEQ ID NO: 1;
b) a second domain comprising an amino acid sequence of SEQ ID NO: 4;
c) a third domain comprising an amino acid sequence of SEQ ID NO: 5;
d) a fourth domain comprising an amino acid sequence of SEQ ID NO: 6; and
e) a fifth domain comprising an amino acid sequence of SEQ ID NO: 7.

17. The method of claim 16, wherein said mammal is a human.

18. The method of claim 16, wherein said first domain further comprises an amino acid sequence of SEQ ID NO: 23.

19. The method of claim 16, wherein said polypeptide comprises an amino acid sequence of SEQ ID NO: 11.

20. The method of claim 16, wherein said nucleic acid comprises a nucleic acid sequence of SEQ ID NO: 17.

21. The method of claim 16, wherein said population comprises immune cells.

22. The method of claim 21, wherein said immune cells are T-cells or Natural Killer (NK) cells.

23. The method of claim 16, wherein said population comprises T-cells.

24. The method of claim 16, wherein said mammal comprises a population of AML cells that are CD64⁺.

25. The method of claim 16, wherein said mammal comprises a population of monocytic leukemia stem cells (mLSCs).

26. The method of claim 16, wherein said mammal has been previously administered at least one AML-targeting therapy.

27. The method of claim 26, wherein said mammal relapsed after treatment with said at least one AML-targeting therapy, or wherein said mammal is resistant to treatment with said at least one AML-targeting therapy.

28. The method of claim 16, wherein said mammal has been previously administered a combination of venetoclax and azacitidine.

29. The method of claim 28, wherein said mammal relapsed after treatment with said combination.

30. The method of claim 28, wherein said mammal is resistant to treatment with said combination.
